(19) United States
(12) Patent Application Publication
(10) Pub. No.: US 2005/0009815 A1
(43) Pub. Date: Jan. 13, 2005

(54) 4-Aminoquinoline compounds

(76) Inventors: Robert J. DeVita, Westfield, NJ (US); Lehua Chang, Ramsey, NJ (US); MyLe Thi Hoang, Colonia, NJ (US); JinLong Jiang, Scotch Plains, NJ (US); Peter Lin, Edison, NJ (US); Andreas W. Sailer, Edison, NJ (US)

Correspondence Address: MERCK AND CO INC, P O BOX 2000, RAHWAY, NJ, 070650907, US

(21) Appl. No.: 10/496,614

(22) PCT Filed: Nov. 22, 2002
(86) PCT No.: PCT/US02/37510

Related U.S. Application Data
(60) Provisional application No. 60/333,464, filed on Nov. 27, 2001.

(51) Int. Cl. A61K 31/541; A61K 31/5377; A61K 31/496; A61K 31/4709

(52) U.S. Cl. 514/227.5; 514/232.5; 514/253.06; 514/313; 544/60; 544/126; 544/363; 546/159

(57) ABSTRACT

The present invention is concerned with compounds of the general Formula I: 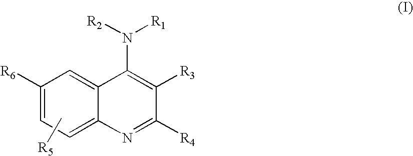 and pharmaceutically acceptable salts thereof, which are useful as melanin concentrating hormone receptor antagonists, particularly MCH-1R antagonists. As such, compounds of the present invention are useful for the treatment or prevention of obesity or eating disorders associated with excessive food intake and complications thereof, osteoarthritis, certain cancers, AIDS wasting, cachexia, frailty (particularly in elderly), mental disorders stress, cognitive disorders, sexual function, reproductive function, kidney function, locomotor disorders, attention deficit disorder (ADD), substance abuse disorders and dyskinesias, Huntington's disease, epilepsy, memory function, and spinal muscular atrophy. Compounds of formula I may therefore be used in the treatment of these conditions, and in the manufacture of a medicament useful in treating these conditions. Pharmaceutical formulations comprising one of the compounds of formula (I) as an active ingredient are disclosed, as are processes for preparing these compounds.

### CROSS-REFERENCE TO RELATED APPLICATIONS

Not applicable.

### BACKGROUND OF THE INVENTION

Obesity, defined as excess adiposity for a given body size, results from a chronic imbalance between energy intake and energy expenditure. Body mass index (BMI, kg/m²) is an accepted clinical estimate of being overweight (BMI 25 to 30) and of obesity (BMI>30). A BMI above 30 kg/m² significantly increases the risk of diabetes, hypertension, dyslipidemias and cardiovascular disease, gallstones, osteoarthritis and certain forms of cancer and reduces life expectancy.

In the vast majority of obese individuals, the cause of the excess adiposity is not immediately apparent. A currently accepted-working hypothesis is that obesity is the result of a maladaptation of the innate metabolic response to environmental challenges such as unlimited availability of low cost/energy dense foods and sedentariness (Hill et al., Science 1998; 280:1371). The study of energy intake in free living humans has met with only limited success and definitive experimental evidence that hyperphagia causes most forms of human obesity is lacking. Following the discovery of leptin, the interest in the neurohormonal regulation of food intake has regained momentum. However, while much knowledge has been gained on the regulation of food intake in rodents and other animal species, the understanding of the neurophysiology of feeding behavior in humans remains extremely limited.

Neuropeptides present in the hypothalamus play a major role in mediating the control of body weight. (Flier, et al., 1998. Cell, 92, 437-440.) Melanin-concentrating hormone (MCH) is a cyclic 19-amino acid neuropeptide synthesized as part of a larger pre-prohormone precursor in the hypothalamus which also encodes neuropeptides NEI and NGE. (Nahon, et al., 1990. Mol. Endocrinol. 4, 632-637.) MCH was first identified in salmon pituitary, and in fish MCH affects melanin aggregation thus affecting skin pigmentation. In trout and in eels MCH has also been shown to be involved in stress induced or CRF-stimulated ACTH release. (Kawauchi, et al., 1983. Nature 305, 321-323.)

In humans two genes encoding MCH have been identified that are expressed in the brain. (Breton, et al., 1993. Mol. Brain Res. 18, 297-310.) In mammals MCH has been localized primarily to neuronal cell bodies of the hypothalamus which are implicated in the control of food intake, including perikarya of the lateral hypothalamus and zona inertia. (Knigge, et al., 1996. Peptides 17, 1063-1073.)

Pharmacological and genetic evidence suggest that the primary mode of MCH action is to promote feeding (orexigenic). MCH mRNA is up-regulated in fasted mice and rats, in the ob/ob mouse and in mice with targeted disruption in the gene, for neuropeptide Y (NPY). (Qu, et al., 1996. Nature 380, 243-247, and Erickson, et al., 1996. Nature 381, 415-418.) Injection of MCH centrally intracelebroventricular (ICV) stimulates food intake and MCH antagonizes the hypophagic effects seen with α melanocyte stimulating hormone (αMSH). (Qu, et al., 1996. Nature 380, 243-247.) MCH deficient mice are lean, hypophagic and have increased metabolic rate. (Shimada, et al., 1998. Nature 396, 670-673.)

MCH action is not limited to modulation of food intake as effects on the hypothalamic-pituitary-axis have been reported. (Nahon, 1994. Critical Rev. in Neurobiol. 8, 221-262.) MCH may be involved in the body response to stress as MCH can modulate the stress-induced release of CRF from the hypothalamus and ACTH from the pituitary.

In addition, MCH neuronal systems may be involved in reproductive or maternal function. MCH transcripts and MCH peptide were found within germ cells in testes of adult rats, suggesting that MCH may participate in stem cell renewal and/or differentiation of early spermatocytes (Hervieu et al., 1996). MCH injected directly into the medial preoptic area (MPOA) or ventromedial nucleus (VMN) stimulated sexual activity in female rats (Gonzalez et al., 1996). In ovariectomized rats primed with estradiol, MCH stimulated luteinizing hormone (LH) release while anti-MCH antiserum inhibited LH release (Gonzalez et al., 1997). The zona incerta, which contains a large population of MCH cell bodies, has previously been identified as a regulatory site for the pre-ovulatory LH surge (MacKenzie et al., 1984). Therefore modulators of MCH receptors may be useful in the prevention and treatment of reproductive function. MCH has been reported to influence release of pituitary hormones including ACTH and oxytocin. Therefore, modulators of MCH receptors may be useful in the prevention and treatment of obesity, Cushing's disease, sexual function, appetite and eating disorders, obesity, diabetes, cardiovascular disease, hypertension, dyslipidemia, myocardial infarction, gall stones, osteoarthritis, certain cancers, AIDS wasting, cachexia, frailty (particularly in the elderly), binge eating disorders including bulimia, anorexia, kidney function, diuresis, reproductive function and sexual function.

Two receptor subtypes have been identified in humans, MCH-1R and MCH-2R. Both receptors, as well as the gene for the MCH peptide, have been mapped to regions previously reported to contain a susceptibility gene for psychiatric disorders. In particular, MCH-1R was mapped to chromosome 22q13.2 (Kolakowski et al. 1996). The possibility of linkage for schizophrenia susceptibility locus in this area was suggested by independent studies from 2 groups (Pulver et al. 1994, Coon et al. 1994). In addition, a more recent study (Stoeber et al. 2000) of samples from patients with periodic catatonia, a clinical subtype of unsystematic schizophrenia suggested possible linkage of the region around 22q13. Human genetics implicates these loci not only for schizophrenia but also for bipolar disorder. The second MCH receptor (MCH-2R) has been mapped to chromosome 6q16.2-16.3 (Sailer et al., 2001). Cao et al. (1997) were the first to report evidence of a schizophrenia susceptibility locus in that area. This initial report was confirmed and extended by other reports (Martinez et al. 1999, Kaufmann et al. 1998, Levinson et al. 2000). Schizophrenia has been recognized as a disorder with profound deficits in information-processing and attentional abnormalities. One of the few possible paradigms available to assess these types of deficits in information processing is sensory gating, a filtering process which can be demonstrated by using a paired auditory stimulus. Miller et al. (1993) examined the effects of ICV administered MCH on the decrease in amplitude of the second of two tone-evoked CNS potentials that can be measured when pairs of identical tones are presented 500 ms apart. They found that MCH application decreased sensory gating in this paradigm. Based on pathogenesis and pathophysiology (reviewed in Lewis and Liebermann (2000)) several brain areas have been implicated in schizophrenia; all of which show high expression for MCH receptors: thalamus, midbrain, nucleus accumbens, temporo-limbic, and prefrontal cortices. These studies and findings support the use of MCH receptor modulators in the treatment and prevention of schizophrenia.

Kelsoe et al. (2001) recently reported on a genome survey indicating a possible susceptibility locus for bipolar disorder identified on 22q (Kelsoe et al. 2001). The MCH gene which encodes the MCH pro-peptide was mapped to chromosome 12q23.1. This area has been identified by Morissette et al. (1999) in a genome wide scan for susceptibility loci for bipolar disorder in families in the Province of Quebec. In addition, Ewald et al. (1998) showed significant linkage to chromosome 12q23.1 (maximum lod score 3.37) in Danish families suffering from bipolar affective disorder. In addition, Presse et al. (1997) have shown that lithium, the “gold standard” and most appropriate initial treatment for the depressive phase of bipolar disorder, can alter MCH MRNA levels in NGF-treated PC12 cells by increasing MRNA stability. These studies and findings support the use of MCH receptor modulators in the treatment and prevention of bipolar disorder and depression.

Philippe and colleagues (1999) performed a genome-wide screen for a autism susceptibility gene and found suggestive linkage for the region of chromosome 6q16.2-16.3 (maximum lod score 2.23). This finding supports the use of MCH receptor modulators in the treatment of autism.

In all species studied to date, a major portion of the neurons of the MCH cell group occupies a rather constant location in those areas of the lateral hypothalamus and subthalamus where they lie and may be a part of some of the so-called “extrapyramidal” motor circuits. These involve substantial striato- and pallidofugal pathways involving the thalamus and cerebral cortex, hypothalamic areas, and reciprocal connections to subthalamic nucleus, substantia nigra, and mid-brain centers (Bittencourt et al., 1992). In their location, the MCH cell group may offer a bridge or mechanism for expressing hypothalamic visceral activity with appropriate and coordinated motor activity. Thus, modulators of MCH receptor function may be useful in the treatment and prevention of movement disorders, such as Parkinson's disease, Parkinson-like syndromes and Huntingdon's Chorea in which extrapyramidal circuits are known to be involved.

Human genetic linkage studies have located authentic hMCH loci on chromosome 12 (12q23-24) and the variant hMCH loci on chromosome 5 (5q12-13) (Pedeutour et al., 1994). Locus 12q23-24 coincides with a locus to which autosomal dominant cerebellar ataxia type II (SCA2 ) has been mapped (Auburger et al., 1992; Twells et al., 1992). This disease comprises neurodegenerative disorders, including an olivopontocerebellar atrophy. Furthermore, the gene for Darier's disease, has been mapped to locus 12q23-24 (Craddock et al., 1993). Dariers' disease is characterized by abnormalities in keratinocyte adhesion and mental illnesses in some families. In view of the functional and neuroanatomical patterns of the MCH neural system in the rat and human brains, the MCH gene may represent a good candidate for SCA2 or Darier's disease. Therefore, modulators of MCH receptors may be useful in the treatment of mental disorders including manic depression, depression, schizophrenia, mood disorders, delirium, dementia, severe mental retardation, anxiety, stress, cognitive disorders, and dyskinesias including Parkinson's disease, Tourette's syndrome, Huntington's disease, cerebellar ataxia, seizures, locomotor disorders, attention deficit disorder (ADD) and substance abuse disorders.

Further, the gene responsible for chronic or acute forms of spinal muscular atrophies has been assigned to chromosome 5q12-13 using genetic linkage analysis (Melki et al., 1990; Westbrook et al., 1992). Therefore, modulators of MCH receptors may be useful in treating muscular dystrophy and dyskinesias, including Parkinson's disease, Tourette's syndrome, Huntington's disease, cerebellar ataxia, and seizures.

Still further, modulators of MCH receptor binding may also be useful in treating epilepsy. In the PTZ seizure model, injection of MCH prior to seizure induction prevented seizure activity in both rats and guinea pigs, suggesting that MCH-containing neurons may participate in the neural circuitry underlying PTZ-induced seizure (Knigge and Wagner, 1997).

MCH has also been observed to affect behavioral correlates of cognitive functions. MCH treatment hastened extinction of the passive avoidance response in rats (McBride et al., 1994), raising the possibility that MCH receptor antagonists may be beneficial for memory storage and/or retention.

A role for MCH in the modulation or perception of pain is supported by the dense innervation of the periaqueductal grey (PAG) by MCH-positive fibers. MCH receptor modulators may be useful as antinociceptives or as analgesics, particularly for the treatment of neuropathic pain.

Finally, MCH may participate in the regulation of fluid intake. ICV infusion of MCH in conscious sheep produced diuretic, natriuretic, and kaliuretic changes in response to increased plasma volume (Parkes, 1996). Together with anatomical data reporting the presence of MCH in fluid regulatory areas of the brain, the results indicate that MCH may be an important peptide involved in the central control of fluid homeostasis in mammals. Therefore, modulators of MCH receptors may be useful in kidney function and diuresis.

PCT publication WO 01/21169 to Takeda discloses MCH antagonists of the structural formula shown below: 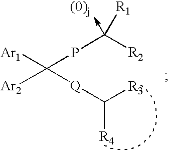
and PCT publication WO 01/21577 discloses MCH antagonists of the structural formula below:

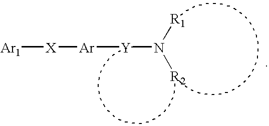

Lanza et al., J. Med. Chem. 1992, 35:252-258, describe substituted 4,6-diaminoquinolines useful as inhibitors of C5a receptor binding. Shinkai, et al., J. Med Chem. 2000, 43:4667-4677, describe 4-aminoquinolines as nociceptin antagonists with analgesic activity.

PCT publication WO 96/28446 discloses N-cycloalkylmethyl-1H-pyrazolo[3,4-b]quinolin-4-amines as inhibitors of cGMP phosphodiesterase and U.S. Pat. No. 5,942,520 claims treating precancerous lesions in mammals with compounds of the structural formula shown below: 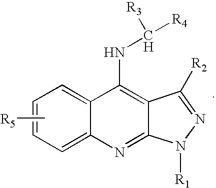
U.S. Pat. No. 4,701,459 and EP 0 252 503 disclose 2,3-dihydro-2-oxo-1H-imidazo[4,5-b]quinolinyl amine derivatives of structural formula: 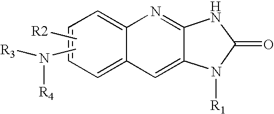
as useful in inhibiting blood platelet aggregation. U.S. Pat. No. 4,013,665 claims antiviral, substituted 1,3-dimethyl-1H-pyrazolo[3,4b]quinolines of structural formula below: 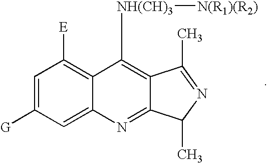
PCT publication WO 99/48492 discloses nociceptin antagonists of the formula below: 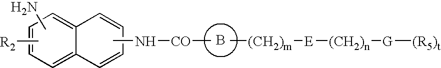
PCT publication WO 99/53924 discloses analgesic agent of the formula below: 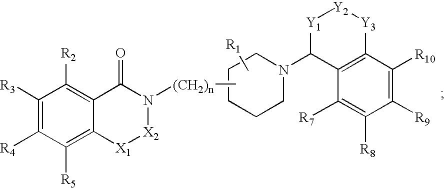
and PCT publication WO 99/19326 discloses compounds of the formula below:

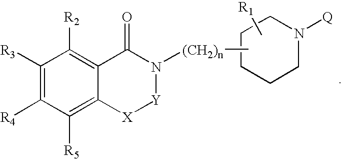

The compounds of the present invention are modulators of the MCH-1R receptor and are useful in the treatment, prevention and suppression of diseases mediated by the MCH-1R receptor. The invention is concerned with the use of these novel compounds to selectively antagonize the MCH-1R receptor. As such, compounds of the present invention are useful for the treatment or prevention of obesity, diabetes, appetite and eating disorders, cardiovascular disease, hypertension, dyslipidemia, myocardial infarction, gall stones, osteoarthritis, certain cancers, AIDS wasting, cachexia, frailty (particularly in elderly), binge eating disorders including bulimina, anorexia, mental disorders including manic depression, depression, schizophrenia, mood disorders, delirium, dementia, severe mental retardation, anxiety, stress, cognitive disorders, sexual function, reproductive function, kidney function, diuresis, locomotor disorders, attention deficit disorder (ADD), substance abuse disorders and dyskinesias including Parkinson's disease, Parkinson-like syndromes, Tourette's syndrome, Huntington's disease, epilepsy, improving memory function, and spinal muscular atrophy.

### SUMMARY OF THE INVENTION

The present invention is concerned with compounds of the general Formula I: 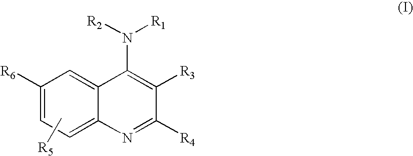
and pharmaceutically acceptable salts thereof, which are useful as melanin concentrating hormone (MCH) receptor antagonists.

As melanin concentrating hormone receptor antagonists, the compounds of the present invention are useful in the treatment, prevention and suppression of diseases mediated by the MCH receptor. In particular, compounds of the present invention are selective antagonists of the MCH-1R subtype receptor. As MCH-1R antagonists, the compounds of the present invention may be useful in treating the following conditions: obesity, diabetes, appetite and eating disorders, cardiovascular disease, hypertension, dyslipidemia, myocardial infarction, gall stones, osteoarthritis, certain cancers, AIDS wasting, cachexia, frailty (particularly in elderly), binge eating disorders including bulirnina, anorexia, mental disorders including manic depression, depression, schizophrenia, mood disorders, delirium, dementia, severe mental retardation, anxiety, stress, cognitive disorders, sexual function, reproductive function, kidney function, diuresis, locomotor disorders, attention deficit disorder (ADD), substance abuse disorders and dyskinesias including Parkinson's disease, Parkinson-like syndromes, Tourette's syndrome, Huntington's disease, epilepsy, improving memory function, and spinal muscular atrophy.

The present invention is also concerned with treatment of these conditions, and the use of compounds of the present invention for manufacture of a medicament useful in treating these conditions.

The invention is also concerned with pharmaceutical formulations comprising one of the compounds as an active ingredient.

The invention is further concerned with processes for preparing the compounds of this invention.

### DETAILED DESCRIPTION OF THE INVENTION

The compounds of this invention are represented by the compound of structural formula I: 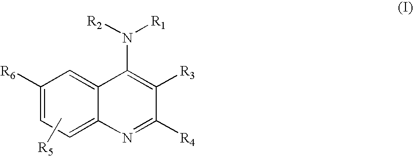
and pharmaceutically acceptable salts thereof.

In one embodiment of the present invention, R¹ is selected from:
(1) hydrogen,
(2) C₁₋₆ alkyl,
(3) C₂₋₆ alkenyl,
(4) C₂₋₆ alkynyl,
(5) cycloalkyl-C₀₋₆ alkyl,
(6) heterocycloalkyl-C₀₋₁₀ alkyl,
(7) aryl-C₀₋₁₀ alkyl, and
(8) heteroaryl-C₀₋₁₀ alkyl;
   wherein alkyl, alkenyl, and alkynyl, moieties above are optionally substituted with one to four substituents independently selected from R^{a}, and cycloalkyl, heterocycloalkyl aryl and heteroaryl moieties above are optionally substituted with one to four substituents independently selected from R^{b}; and wherein sulfur-containing heterocyclic rings may be mono- or di-oxidized on the sulfur atom.

In one class of this embodiment of the present invention, R¹ is selected from:
(1) hydrogen,
(2) C₁₋₆ alkyl,
(3) C₂₋₆ alkenyl,
(4) cycloalkyl-C₀₋₆ alkyl,
(5) heterocycloalkyl-C₀₋₆ alkyl,
(6) aryl-C₀₋₆ alkyl, and
(7) heteroaryl-C₀₋₁₀ alkyl;
   wherein alkyl and alkenyl moieties above are optionally substituted with one to three substituents independently selected from R^{a}, and cycloalkyl, heterocycloalkyl, aryl and heteroaryl moieties above are optionally substituted with one to three substituents independently selected from R^{b}.

In one subclass of this class of the invention, R¹ is hydrogen, or C₁₋₆ alkyl, optionally substituted with one to three substituents independently selected from R^{a}.

In another subclass of this class of the invention, R¹ is selected from:
(1) hydrogen,
(2) methyl,
(3) ethyl, and
(4) propyl,
   optionally substituted with one to three substituents independently selected from R^{a}.

In one embodiment of the present invention, R² is selected from:
(1) hydrogen,
(2) C₁₋₆ alkyl,
(3) C₂₋₆ alkenyl,
(4) C₂₋₆ alkynyl,
(5) cycloalkyl-C₀₋₆ alkyl,
(6) heterocycloalkyl-C₀₋₁₀ alkyl,
(7) aryl-C₀₋₁₀ alkyl, and
(8) heteroaryl-C₀₋₁₀ alkyl;
   wherein alkyl, alkenyl, and alkynyl, moieties above are optionally substituted with one to four substituents independently selected from R^{a}, and cycloalkyl, heterocycloalkyl aryl and heteroaryl moieties above are optionally substituted with one to four substituents independently selected from R^{b}; and wherein sulfur-containing heterocyclic rings may be mono- or di-oxidized on the sulfur atom.

In one class of this embodiment of the present invention, R² is selected from:
(1) hydrogen,
(2) C₁₋₆ alkyl,
(1) C₂₋₆ alkenyl,
(2) cycloalkyl-C₀₋₆ alkyl,
(3) heterocycloalkyl-C₀₋₆ alkyl,
(4) aryl-C₀₋₆ alkyl, and
(5) heteroaryl-C₀₋₁₀ alkyl;
   wherein alkyl and alkenyl moieties above are optionally substituted with one to three substituents independently selected from R^{a}, and cycloalkyl, heterocycloalkyl, aryl and heteroaryl moieties above are optionally substituted with one to three substituents independently selected from R^{b}.

In one subclass of this class, R² is selected from:
(1) hydrogen,
(2) C₁₋₆ alkyl,
(3) cycloalkyl-C₀₋₆ alkyl,
(4) heterocycloalkyl-C₀₋₆ alkyl,
(5) aryl-C₀₋₆ alkyl, and
(6) heteroaryl-C₀₋₁₀ alkyl;
   wherein alkyl moieties above are optionally substituted with one to three substituents independently selected from R^{a}, and cycloalkyl, heterocycloalkyl, aryl and heteroaryl moieties above are optionally substituted with one to three substituents independently selected from R^{b}.

In another subclass of this class of the invention, R² is selected from:
(1) hydrogen,
(2) C₁₋₆ alkyl,
(3) cycloalkyl-C₀₋₆ alkyl,
(4) heterocycloalkyl-C₀₋₆ alkyl, and
(5) aryl-C₀₋₆ alkyl,
   wherein alkyl moieties above are optionally substituted with one to three substituents independently selected from R^{a}, and cycloalkyl, heterocycloalkyl, aryl and heteroaryl moieties above are optionally substituted with one to three substituents independently selected from R^{b}.

In yet another subclass of this class of the invention, R² is selected from the group consisting of:
(1) hydrogen,
(2) methyl,
(3) ethyl,
(4) n-propyl,
(5) isopropyl,
(6) t-butyl,
(7) n-butyl,
(8) cyclopropyl,
(9) cyclobutyl,
(10) cyclopentyl,
(11) cyclohexyl,
(12) heterocycloalkyl-C₀₋₆ alkyl, wherein the heterocycloalkyl moiety is selected from azetidinyl, pyrrolidinyl, and pyridyl, and
(13) phenyl-C₀₋₃alkyl,
   wherein alkyl moieties above are optionally substituted with one to three substituents independently selected from R^{a}, and cycloalkyl, heterocycloalkyl, aryl and heteroaryl moieties above are optionally substituted with one to three substituents independently selected from R^{b}.

In another embodiment of the present invention, R¹ and R² together with the nitrogen atom to which they are attached, form a 4- to 10-membered bridged or unbridged heterocyclic ring, optionally containing one or two additional heteroatoms selected from N, S, and O, optionally having one or more degrees of unsaturation, optionally fused to a 6-membered heteroaromatic or aromatic ring, either unsubstituted or substituted with one to four substituents independently selected from R^{b}; and wherein sulfur-containing heterocyclic rings may be mono- or di-oxidized on the sulfur atom. In one class of this embodiment of the invention, R¹ and R² together with the nitrogen atom to which they are attached, form a 4- to 10-membered bridged or unbridged heterocyclic ring, optionally containing one additional heteroatom selected from N, S, and O optionally having one or more degrees of unsaturation, optionally fused to a 6-membered heteroaromatic or aromatic ring, either unsubstituted or substituted with an R^{b} substituent. In one subclass of this class, R¹ and R² together with the nitrogen atom to which they are attached, form a 4- to 10-membered bridged or unbridged heterocyclic ring, optionally containing one additional heteroatom selected from N, S, and O, either unsubstituted or substituted with an R^{b} substituent. In yet another subclass of the present invention, R¹ and R² together with the nitrogen atom to which they are attached, form a 4- to 10-membered bridged or unbridged heterocyclic ring, selected from: azetidinyl, pyrrolidinyl, piperidinyl, morpholinyl, 1-thia-4-azacyclohexyl, azacycloheptyl, 2-oxa-5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, either unsubstituted or substituted with an R^{b} substituent. In still another subclass of the present invention, R¹ and R² together with the nitrogen atom to which they are attached, form a 4- to 6-membered unbridged heterocyclic ring, selected from: azetidinyl, pyrrolidinyl, piperidinyl, either unsubstituted or substituted with an R^{b} substituent.

In yet another embodiment of this invention, R¹ and R² together with the nitrogen atom to which they are attached, are selected from: unsubstituted amino, N-methylamino, N-ethylamino, N,N-dimethylamino, N,N-diethylamino, N-cyclopropylamino, N-cyclobutylamino, azetidinyl, pyrrolidinyl, piperidinyl, and 4-(4-fluorophenyl)piperidinyl.

In yet another embodiment of the present invention, R³ is selected from the group consisting of:
(1) hydrogen,
(2) halogen,
(3) C₁₋₈alkyl,
(4) perfluoro C₁₋₆ alkyl,
(5) C₂₋₆ alkenyl,
(6) C₂₋₆ alkynyl,
(7) cycloalkyl,
(8) cycloalkyl-C₁₋₆ alkyl,
(9) cycloheteroalkyl,
(10) cycloheteroalkyl-C₁₋₆ alkyl,
(11) aryl,
(12) aryl-C₁₋₆ alkyl,
(13) heteroaryl,
(14) heteroaryl-C₁₋₆ alkyl,
(15) —OR⁷,
(16) —NR⁷R⁷,
(17) —CO₂R⁷,
(18) cyano, and
(19) —C(O)NR⁷R⁷;
   wherein alkyl, alkenyl and alkynyl, moieties above are optionally substituted with one to four substituents independently selected from R^{a}, and cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom.

In one class of this embodiment of the present invention, R³ is selected from:
(1) hydrogen,
(2) halogen,
(3) C₁₋₈ alkyl,
(4) trifluoromethyl,
(5) C₂₋₆ alkenyl,
(6) cycloalkyl,
(7) cycloalkyl-C₁₋₆ alkyl,
(8) cycloheteroalkyl,
(9) cycloheteroalkyl-C₁₋₆ alkyl,
(10) aryl,
(11) aryl-C₁₋₆ alkyl,
(12) heteroaryl,
(13) heteroaryl-C₁₋₆ alkyl,
(14) —OR⁷,
(15) —NR⁷R⁷,
(16) —CO₂R⁷, and
(17) —C(O)NR⁷R⁷;
   wherein alkyl and alkenyl moieties above are optionally substituted with one to three substituents independently selected from R^{a}, and cycloalkyl, heterocycloalkyl, aryl and heteroaryl moieties above are optionally substituted with an R^{b} substituent.

In one subclass of this class, R³ is selected from:
(1) hydrogen,
(2) halogen,
(3) C₁₋₈ alkyl,
(4) trifluoromethyl,
(5) —OH,
(6) —OCH₃,
(7) —NH₂,
(8) —CO₂R⁷, and
(9) —C(O)NH₂;
   wherein alkyl moieties above are optionally substituted with one to two substituents independently selected from R^{a}.

In another subclass of this class, R³ is selected from:
(1) hydrogen,
(2) halogen,
(3) C₁₋₈ alkyl,
(4) trifluoromethyl,
(5) —OH,
(6) —OCH₃,
(7) —NH₂,
(8) —CO₂H,
(9) —CO₂CH₃,
(10) —CO₂CH₂CH₃, and
(11) —C(O)NH₂;
   wherein alkyl moieties above are optionally substituted with one to three substituents independently selected from R^{a}.

In yet another subclass of this class, R³ is selected from:
(1) hydrogen,
(2) halogen,
(3) C₁₋₈ alkyl,
(4) trifluoromethyl,
(5) —OH,
(6) —OCH₃,
(7) —NH₂,
(8) —CO₂H,
(9) —CO₂CH₃, and
(10) —CO₂CH₂CH₃;
   wherein alkyl moieties above are optionally substituted with one to three substituents independently selected from R^{a}.

In still another subclass of this class, R³ is selected from hydrogen and —CO₂CH₂CH₃. In yet another subclass, R³ is hydrogen.

In still another embodiment of the present invention, R⁴ is selected from the group consisting of:
(1) hydrogen,
(2) halogen,
(3) C₁₋₈ alkyl,
(4) perfluoro C₁₋₆ alkyl,
(5) C₂₋₆ alkenyl,
(6) C₂₋₆ alkynyl,
(7) cycloalkyl,
(8) cycloalkyl-C₁₋₆ alkyl,
(9) cycloheteroalkyl,
(10) cycloheteroalkyl-C₁₋₆ alkyl,
(11) aryl,
(12) aryl-C₁₋₆ alkyl,
(13) heteroaryl,
(14) heteroaryl-C₁₋₆ alkyl,
(15) —OR⁷,
(16) —NR⁷R⁷,
(17) —CO₂R⁷, and
(18) —C(O)NR⁷R⁷;
   wherein alkyl, alkenyl and alkynyl, moieties above are optionally substituted with one to four substituents independently selected from R^{a}, and cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom.

In one class of this embodiment of the present invention, R⁴ is selected from:
(1) hydrogen,
(2) halogen,
(3) C₁₋₈ alkyl,
(4) trifluoromethyl,
(5) C₂₋₆ alkenyl,
(6) cycloalkyl,
(7) cycloalkyl-C₁₋₆ alkyl,
(8) cycloheteroalkyl,
(9) cycloheteroalkyl-C₁₋₆ alkyl,
(10) aryl,
(11) aryl-C₁₋₆ alkyl,
(12) heteroaryl,
(13) heteroaryl-C₁₋₆ alkyl,
(14) —OR⁷,
(15) —NR⁷R⁷,
(16) —CO₂R⁷, and
(17) —C(O)NR⁷R⁷;
   wherein alkyl and alkenyl moieties above are optionally substituted with one to three substituents independently selected from R^{a}, and cycloalkyl, heterocycloalkyl, aryl and heteroaryl moieties above are optionally substituted with an R^{b}substituent.

In one subclass of this class of the invention, R⁴ is selected from the group consisting of:
(1) hydrogen,
(2) halogen,
(3) C₁₋₈ alkyl,
(4) trifluoromethyl,
(5) cycloalkyl,
(6) cycloheteroalkyl,
(7) aryl,
(8) aryl-C₁₋₆ alkyl,
(9) heteroaryl,
(10) —OH,
(11) —OCH,
(12) —NH₂,
(13) —CO₂R⁷, and
(14) —C(O)NH₂;
   wherein alkyl moieties above are optionally substituted with one to four substituents independently selected from R^{a}, and cycloalkyl, heterocycloalkyl, aryl and heteroaryl moieties above are optionally substituted with an R^{b} substituent.

In another subclass of this class of the invention, R⁴ is selected from:
(1) C₁₋₈ alkyl,
(2) trifluoromethyl,
(3) cycloalkyl,
(4) cycloheteroalkyl,
(5) aryl,
(6) heteroaryl,
(7) —NH₂,
(8) —CO₂H,
(9) —CO₂CH₃, and
(10) —CO₂CH₂CH₃;
   wherein alkyl moieties above are optionally substituted with one to three substituents independently selected from R^{a}, and cycloalkyl, heterocycloalkyl, aryl and heteroaryl moieties above are optionally substituted with an R^{b} substituent.

In yet another subclass of this class, R⁴ is selected from:
(1) C₁₋₈ alkyl,
(2) trifluoromethyl,
(3) cyclobutyl,
(4) cyclopentyl,
(5) cyclohexyl,
(6) phenyl,
(7) —CO₂H,
(8) CO₂CH₃, and
(9) —CO₂CH₂CH₃;
   wherein alkyl moieties above are optionally substituted with one to three substituents independently selected from R^{a}, and cycloalkyl, heterocycloalkyl, aryl and heteroaryl moieties above are optionally substituted with an R^{b} substituent.

In still another subclass of this class, R⁴ is selected from: methyl, ethyl, n-propyl, isopropyl, n-butyl, t-butyl, 2,2-dimethylpropyl, 1-methylpropyl, n-pentyl, n-hexyl, phenyl, methoxymethyl, methylthiomethyl, cyclobutyl, cyclopentyl, and cyclohexyl.

In one embodiment of the present invention, R³ and R⁴ are not both hydrogen.

In another embodiment of the present invention, R³ and R⁴ together with the ring carbon atoms to which they are attached, form a 5- to 7-membered heterocycloalkyl or cycloalkyl ring, either unsubstituted or substituted with one to four substituents independently selected from R^{b}. In one class of this embodiment of the present invention, R³ and R⁴ together with the ring carbon atoms to which they are attached, form a 5- to 7-membered heterocycloalkyl or cycloalkyl ring, either unsubstituted or substituted with an R^{b} substituent. In one subclass of this embodiment, R³ and R⁴ together with the ring carbon atoms to which they are attached, form a 5- to 7-membered cycloalkyl ring, either unsubstituted or substituted with oxo or hydroxy. In another subclass of this class, R³ and R⁴ together with the ring carbon atoms to which they are attached, form a cyclohexyl ring, either unsubstituted or substituted with oxo or hydroxy.

In one embodiment of the present invention, R⁵ is selected from:
(1) hydrogen,
(2) halogen,
(3) C₁₋₆ alkyl,
(4) perfluoro C₁₋₆ alkyl,
(5) —OR⁷, and
(6) —NR⁷R⁷.

In one class of this embodiment of the present invention, R⁵ is selected from:
(1) hydrogen,
(2) halogen,
(3) methyl,
(4) trifluoromethyl,
(5) hydroxy,
(6) methoxy,
(7) phenoxy,
(8) —NH₂,
(9) —NH(CH₃), and
(10) —N(CH₃)₂.

In one class of this embodiment of the invention, R⁵ is selected from:
(1) hydrogen,
(2) halogen,
(3) methyl,
(4) trifluoromethyl,
(5) hydroxy,
(6) methoxy,
(7) phenoxy,
(8) —NH₂,
(9) —NH(CH₃), and
(10) —N(CH₃)₂.

In one subclass of this invention, R⁵ is selected from:
(1) hydrogen,
(2) halogen,
(3) methyl,
(4) trifluoromethyl,
(5) hydroxy, and
(6) methoxy.

In another subclass of this invention, R⁵ is hydrogen.

In another embodiment of the present invention, R⁶ is selected from:
(1) —(CH₂)ₙ—R⁷,
(2) —(CH₂)ₙ-aryl-R⁷,
(3) —(CH₂)ₙ-heteroaryl-R⁷,
(4) —(CH₂)ₙ-heterocycloalkyl-R⁷,
(5) —(CH₂)ₙC≡N,
(6) —(CH₂)ₙCON(R⁷)₂,
(7) —(CH₂)ₙCO₂R⁷,
(8) —(CH₂)ₙCOR⁷,
(9) —(CH₂)ₙNR⁷C(O)R⁷,
(10) —(CH₂)ₙNR⁷C(O)(CH₂)ₙSR⁷
(11) —(CH₂)ₙNR⁷CO₂R⁷,
(12) —(CH₂)ₙNR⁷C(O)N(R⁷)₂,
(13) —(CH₂)ₙNR⁷SO₂R⁷,
(14) —(CH₂)ₙS(O)ₚR⁷,
(15) —(CH₂)ₙSO₂N(R⁷)₂,
(16) —(CH₂)ₙOR⁷,
(17) —(CH₂)ₙOC(O)R⁷,
(18) —(CH₂)ₙOC(O)OR⁷,
(19) —(CH₂)ₙOC(O)N(R⁷)₂,
(20) —(CH₂)ₙN(R⁷)₂, and
(21) —(CH₂)ₙNR⁷SO₂N(R⁷)₂,
   wherein one or two of the hydrogen atoms in (CH₂)ₙ may be substituted with R^{a}.

In one class of this invention, R⁶ is selected from:
(1) —(CH₂)ₙ—R⁷,
(2) —(CH₂)ₙ-aryl-R⁷,
(3) —(CH₂)ₙ-heteroaryl-R⁷,
(4) —(CH₂)ₙ-heterocycloalkyl-R⁷,
(5) —(CH₂)ₙC≡N,
(6) —(CH₂)ₙCON(R⁷)₂,
(7) —(CH₂)ₙCO₂R⁷, provided that n is 1, 2, 3, 4, or 5,
(8) —(CH₂)ₙCOR⁷,
(9) —(CH₂)ₙNR⁷C(O)R⁷, provided that n is 1, 2, 3, 4, or 5,
(10) —(CH₂)ₙNR⁷C(O)(CH₂)ₙSR⁷
(11) —(CH₂)ₙNR⁷CO₂R⁷,
(12) —(CH₂)ₙNR⁷C(O)N(R⁷)₂,
(13) —(CH₂)ₙNR⁷SO₂R⁷, provided that n is 1, 2, 3, 4, or 5,
(14) —(CH₂)ₙS(O)ₚR⁷,
(15) —(CH₂)ₙSO₂N(R⁷)₂,
(16) —(CH₂)ₙOR⁷,
(17) —(CH₂)ₙOC(O)R⁷,
(18) —(CH₂)ₙOC(O)OR⁷,
(19) —(CH₂)ₙOC(O)N(R⁷)₂,
(20) —(CH₂)ₙN(R⁷)₂, provided that when n is zero, at least one R⁷ is other than hydrogen, phenyl and alkyl, and
(21) —(CH₂)ₙNR⁷SO₂N(R⁷)₂,
   wherein one or two of the hydrogen atoms in (CH₂)ₙ may be substituted with R^{a}.

In one class of the present invention, R⁶ is selected from:
(1) —(CH₂)ₙ—R⁷,
(2) —(CH₂)ₙ-aryl-R⁷,
(3) —(CH₂)ₙ-heteroaryl-R⁷,
(4) —(CH₂)ₙ-heterocycloalkyl-R⁷,
(5) —(CH₂)ₙCON(R⁷)₂,
(6) —(CH₂)ₙNR⁷C(O)R⁷,
(7) —(CH₂)ₙNR⁷C(O)(CH₂)ₙSR⁷
(8) —(CH₂)ₙNR⁷C(O)N(R⁷)₂,
(9) —(CH₂)ₙNHSO₂R⁷,
(10) —(CH₂)ₙN(R⁷)₂, and
(11) —(CH₂)ₙNR₇SO₂N(R⁷)₂,
   wherein one or two of the hydrogen atoms in (CH₂)ₙ may be substituted-with R^{a}.

In another class of the present invention, R⁶ is selected from:
(1) —R⁷,
(2) -heteroaryl-R⁷,
(3) —CONHR⁷,
(4) —CON(R⁷)(CH₃),
(5) —CH₂CONHR⁷,
(6) —CH₂CON(R⁷)(CH₃),
(7) —CH₂NHC(O)R⁷,
(8) —NHC(O)R⁷,
(9) —(CH₂)ₙNHC(O)(CH₂)ₙSR⁷
(10) —(CH₂)ₙNHC(O)N(CH₃)(R⁷),
(11) —(CH₂)ₙNHC(O)NH(R⁷),
(12) —(CH₂)ₙNHSO₂R⁷,
(13) —NH(R⁷),
(14) —N(COCH₃)(R⁷),
(15) —(CH₂)ₙNH(R⁷), and
(16) —(CH₂)ₙN(COCH³)(R⁷),
   wherein one or two of the hydrogen atoms in (CH₂)ₙ may be substituted with R^{a}.

In a particular subclass of the present invention, R⁶ is -oxadiazolyl-R⁷.

In yet another embodiment of the present invention, R⁷ is independently selected at each occurrence from the group consisting of:
(1) hydrogen,
(2) C₁₋₆ alkyl,
(3) aryl,
(4) heteroaryl,
(5) cycloalkyl,
(6) heterocycloalkyl,
(7) aryl C₁₋₃ alkyl,
(8) heteroaryl C₁₋₃ alkyl,
(9) cycloalkyl C₁₋₃ alkyl,
(10) heterocycloalkyl C₁₋₃ alkyl,
(11) aryl C₂₋₃ alkenyl,
(12) heteroaryl C₂₋₃ alkenyl,
(13) cycloalkyl C₂₋₃ alkenyl, and
(14) heterocycloalkyl C₂₋₃ alkenyl,
   wherein the alkyl and alkenyl moieties are optionally substituted with one to four substituents selected from R^{a}, and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom. In one class of the compounds of the present invention, in R⁷, the alkyl and alkenyl moieties are optionally substituted with one to three substituents selected from R^{a}, and wherein the aryl, heteroaryl, cycloalkyl and heterocycloalkyl moieties are independently substituted with one to three substituents selected from R^{b}; and wherein sulfur-containing heterocyclic rings may be mono- or di-oxidized on the sulfur atom.

In one class of the present invention, R⁷ is independently selected at each occurrence from:
(1) hydrogen,
(2) C₁₋₆ alkyl,
(3) aryl, selected from: phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, and 1,4-benzodioxanyl,
(4) heteroaryl, selected from: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyriridinyl, 5,6,7,8-tetrahydroquinolinyl, 2,1,3-benzothiadiazolyl, and thienopyridinyl,
(5) cycloalkyl, selected from: cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl,
(6) heterocycloalkyl, selected from: azetidinyl, pyridyl, pyrrolidinyl, piperidinyl, piperazinyl, imidazolidinyl, morpholinyl, 1-thia-4-aza-cyclohexane, 2,5-diazabicyclo[2.2.2]octane, 2,3-dihydrofuro[2,3-b]pyridyl, benzoxazinyl, tetrahydroquinolinyl, tetrahydroisoquinolinyl, dihydroindolyl, indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl,
(7) aryl C₁₋₃ alkyl, wherein the aryl moiety is selected from: phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3dihydrobenzofuranyl, dihydrobenzopyranyl, and 1,4-benzodioxanyl,
(8) heteroaryl C₁₋₃ alkyl, wherein the heteroaryl moiety is selected: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyriridinyl, 5,6,7,8-tetrahydroquinolinyl, 2,1,3-benzothiadiazolyl, and thienopyridinyl,
(9) cycloalkyl C₁₋₃ alkyl, wherein the cycloalkyl moiety is selected from: cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl,
(10) heterocycloalkyl C₁₋₃ alkyl, wherein the heterocycloalkyl moiety is selected from: azetidinyl, pyridyl, pyrrolidinyl, piperidinyl, piperazinyl, imidazolidinyl, morpholinyl, 1-thia4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl,indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl,
(11) aryl C₂₋₃ alkenyl, wherein the aryl moiety is selected from: phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, and 1,4-benzodioxanyl,
(12) heteroaryl C₂₋₃ alkenyl, wherein the heteroaryl moiety is selected from: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, and thienopyridinyl,
(13) cycloalkyl C₂₋₃ alkenyl, wherein the cycloalkyl moiety is selected from: cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl, and
(14) heterocycloalkyl C₂₋₃ alkenyl, wherein the heterocycloalkyl moiety is selected from: azetidinyl, pyridyl, pyrrolidinyl, piperidinyl, piperazinyl, imidazolidinyl, morpholinyl, 1-thia-4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl, indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl;
   wherein the alkyl moieties are optionally substituted with one to three substituents selected from R^{a}, and wherein the aryl, heteroaryl, cycloalkyl and heterocycloalkyl moieties are independently substituted with one to three substituents selected from R^{b}; and wherein sulfur-containing heterocyclic rings may be mono- or di-oxidized on the sulfur atom;

In another embodiment of the present invention, R^{a} is independently selected from:
(1) —OR^{d},
(2) —NR^{d}S(O)ₘR^{d},
(3) —NO₂,
(4) halogen,
(5) —S(O)ₘR^{d},
(6) —SR^{d},
(7) —S(O)₂OR^{d},
(8) —S(O)ₚN(R^{d})₂,
(9) —N(R^{d})₂,
(10) —O(CR^{d}R^{d})ₙN(R^{d})₂,
(11) —C(O)R^{d},
(12) —CO₂R^{d},
(13) —CO₂(CR^{d}R^{d})ₙCON(R^{d})₂,
(14) —OC(O)R^{d},
(15) —CN,
(16) —C(O)N(R^{d})₂,
(17) —NR^{d}C(O)R^{d},
(18) —OC(O)N(R^{d})₂,
(19) —NR^{d}C(O)OR^{d},
(20) —NR^{d}C(O)N(R^{d})₂,
(21) —CR^{d}(N—OR^{d}),
(22) —CF₃,
(23) cycloalkyl,
(24) cycloheteroalkyl, and
(25) oxo;
   at each occurrence.

In one class of this-embodiment of the present invention, R^{a} is independently selected from:
(1) —OR^{d},
(2) —NHSO₂CH₃,
(3) —NO₂,
(4) halogen,
(5) —S(O)ₘCH₃,
(6) —SR^{d},
(7) —S(O)₂OR^{d},
(8) S(O)ₚN(R^{d})₂,
(9) —N(R^{d})₂,
(10) —O(CR^{d}R^{d})ₙN(R^{d})₂,
(11) —C(O)R^{d},
(12) —CO₂R^{d},
(13) —CO₂(CR^{d}R^{d})ₙCON(R^{d})₂,
(14) —OC(O)R^{d},
(15) —CN,
(16) —C(O)N(R^{d})₂,
(17) —NR^{d}C(O)R^{d},
(18) —OC(O)N(R^{d})₂,
(19) —NR^{d}C(O)OR^{d},
(20) —NR^{d}C(O)N(R^{d})₂,
(21) —CR^{d}(N—OR^{d}),
(22) —CF₃,
(23) cycloalkyl,
(24) cycloheteroalkyl, and
(25) oxo;
   at each occurrence.

In a subclass of this class of the invention, R^{a} is independently selected from:
(1) —OR^{d},
(2) —NHSO₂CH₃,
(3) —NO₂,
(4) halogen,
(5) —S(O)ₘCH₃,
(6) —SCH₃,
(7) —SCF₃,
(8) —S(O)₂OH,
(9) —S(O)ₚN(R^{d})₂,
(10) —N(CH₃)₂,
(11) —NH₂,
(12) —O(CR^{d}R^{d})ₙN(R^{d})₂,
(13) —C(O)R^{d},
(14) —CO₂H,
(15) —CO₂CH₃,
(16) t-butyloxycarbonyl,
(17) —CO₂(CR^{d}R^{d})ₙCON(R^{d})₂,
(18) —OC(O)R^{d},
(19) —CN,
(20) —C(O)N(R^{d})₂,
(21) —NR^{d}C(O)R^{d},
(22) —OC(O)N(R^{d})₂,
(23) —NR^{d}C(O)OR^{d},
(24) —NR^{d}C(O)N(R^{d})₂,
(25) —CR^{d}(N—OR^{d}),
(26) —CF₃,
(27) cycloalkyl,
(28) cycloheteroalkyl, and
(29) oxo;
   at each occurrence.

In another embodiment of the present invention, each R^{b} is independently selected from:
(1) R^{a},
(2) —Sn(CH₃)₃,
(3) C₁₋₁₀ alkyl,
(4) C₂₋₁₀ alkenyl,
(5) C₂₋₁₀ alkynyl,
(6) heteroaryl,
(7) aryl,and
(8) aryl-C₁₋₁₀ alkyl;
   wherein alkyl, alkenyl, alkynyl, cycloalkyl, cycloheteroalkyl, heteroaryl, and aryl are optionally substituted with one to four substituents selected from a group independently selected from R^{c}.

In one class of this embodiment of the present invention, each R^{b} is independently selected from:
(1) R^{a},
(2) —Sn(CH₃)₃,
(3) C₁₋₁₀ alkyl,
(4) C₂₋₁₀ alkenyl,
(5) heteroaryl,
(6) aryl, and
(7) aryl-C₁₋₁₀ alkyl;
   wherein alkyl, alkenyl, cycloalkyl, cycloheteroalkyl, heteroaryl, and aryl are optionally substituted with one to four substituents selected from a group independently selected from R^{c}.

In one subclass of this class of the invention, each R^{b} is independently selected from:
(1) R^{a},
(2) —Sn(CH₃)₃,
(3) C₁₋₆ alkyl,
(4) C₂₋₆ alkenyl,
(5) heteroaryl,
(6) aryl, and
(7) aryl-C₁₋₁₀ alkyl;
   wherein alkyl, alkenyl, cycloalkyl, cycloheteroalkyl, heteroaryl, and aryl moieties in R^{a} and R^{b} are optionally substituted with one to four substituents selected from a group independently selected from R^{c}.

In another subclass of the present invention, each R^{b} is independently selected from:
(1) —R^{a},
(2) —Sn(CH₃)₃,
(3) C₁₋₆ alkyl,
(4) C₂₋₆ alkenyl,
(5) heteroaryl,
(6) phenyl, and
(7) phenyl-C₁₋₁₀ alkyl;
   wherein alkyl, alkenyl, cycloalkyl, cycloheteroalkyl, heteroaryl, and aryl moieties in R^{a} and R^{b} are optionally substituted with one to four substituents selected from a group independently selected from R^{c}.

In yet another embodiment of the present invention, each R^{c} is independently selected from:
(1) halogen,
(2) amino,
(3) carboxy,
(4) C₁₋₄ alkyl,
(5) C₁₋₄ alkoxy,
(6) aryl,
(7) aryl C₁₋₄ alkyl,
(8) hydroxy,
(9) —CF₃,
(10) —OC(O)C₁₋₄ alkyl,
(11) —OC(O)N(R^{d})₂, and
(12) aryloxy.

In still another embodiment of the present invention, R^{d} is independently selected from hydrogen, C₁₋₆ alkyl, C₂₋₆ alkenyl; C₂₋₆ alkynyl; cycloalkyl; cycloalkyl-C₁₋₆ alkyl; cycloheteroalkyl; cycloheteroalkyl-C₁₋₆ alkyl; aryl; heteroaryl; aryl-C₁₋₆ alkyl; and heteroaryl-C₁₋₆ alkyl; wherein the alkyl, alkenyl, alkynyl, cycloalkyl, cycloheteroalkyl, heteroaryl, and aryl in R^{d} are optionally substituted with one to four substituents independently selected from R^{e}. In one class of this embodiment of the present invention, the alkyl, alkenyl, alkynyl, cycloalkyl, cycloheteroalkyl, heteroaryl, and aryl in R^{d} are optionally substituted with one to two substituents independently selected from a R^{e}.

In another embodiment of the present invention, each R^{e} is selected from halo, methyl, methoxy, trifluoromethyl, trifluoromethoxy, and hydroxy.

In still another embodiment of the present invention, each m is independently selected from 1 and 2. In one class of this embodiment, m is 1. In another class of this embodiment m is 2.

In yet another embodiment of the present invention, n is independently elected from 0, 1, 2, 3, 4, and 5 at each occurrence. In one class of this embodiment, each n is independently selected from 0, 1, 2, 3, and 4. In one subclass of this class, n is selected from 0, 1, 2, and 3. In another subclass of this class, n is selected from 0, 1, and 2. In still another subclass of this class, n is 0.

In still another embodiment of the present invention, each p is independently selected from 0, 1, and 2. In one class of this embodiment, p is 0. In another class of this embodiment, p is 1. In still another class of this embodiment, p is 2.

“Alkyl”, as well as other groups having the prefix “alk”, such as alkoxy, alkanoyl, means carbon chains which may be linear or branched or combinations thereof. Examples of alkyl groups include methyl, ethyl, n-propyl, isopropyl, n-butyl, 1-methylpropyl, 2-methylpropyl, Lert-butyl, n-pentyl, 1-methylbutyl, 2-methylbutyl, 3-methylbutyl, 1,2-dimethylpropyl, 1,1-dimethylpropyl, 2,2-dimethylpropyl, n-hexyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethyl butyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, 4-ethylpentyl, 1-propylbutyl, 2-propylbutyl, 3-propylbutyl, 1,1-dimethylpentyl, 1,2-dimethylpentyl, 1,3-dimethylpentyl, 1,4-dimethylpentyl, 2,2-dimethylpentyl, 2,3-dimethylpentyl. 2,4-dimethylpentyl, 3,3-dimethylpentyl, 3,4-dimethylpentyl, 4,4-dimethylpentyl, 1-methyl-1-ethylbutyl, 1-methyl-2-ethylbutyl, 2-methyl-2-ethylbutyl, 1-ethyl-2-methylbutyl, 1-ethyl-3-methylbutyl, 1,1-diethylpropyl, n-octyl, n-nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, dihydroindanyl, 3,3-spirohexylindoline, 5,6,7,8-tetrahydroquinoline, and the like.

“Aryl” means mono- or bicyclic aromatic rings containing only carbon atoms. The term also includes aryl group fused to a monocyclic cycloalkyl or monocyclic heterocycloalkyl group in which the point of attachment is on the aromatic portion. Examples of aryl include phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, 1,4-benzodioxanyl, and the like.

“Heteroaryl” means a mono- or bicyclic aromatic ring containing at least one heteroatom selected from N, O and S, with each ring containing 5- to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, thienopyridinyl, and the like.

“Heterocycloalkyl” means mono- or bicyclic saturated rings containing at least one heteroatom selected from N, S and O, each of said ring having from 3 to 14 atoms in which the point of attachment may be carbon or nitrogen. The term also refers to bridged rings, and also includes monocyclic heterocycles fused to an aryl or heteroaryl group in which the point of attachment is on the non-aromatic portion. Examples of “heterocycloalkyl” include azetidinyl, pyridyl, pyrrolidinyl, piperidinyl, piperazinyl, imidazolidinyl, morpholinyl, 1-thia4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl,indolyl, indolinyl, isoindolinyl, 1,3-dihydro-2-benzofuranyl, benzodioxolyl, hexahydrothienopyridinyl, thienopyridinyl, azacycloheptyl, 2-oxa-5-azabicyclo[2.2.1]heptyl, 2,5-diazabicyclo[2.2.1]heptyl, 2-azabiclyclo[2.2.1]heptyl, 7-azabicyclo[2.2.1.]heptyl, 2,4-dizaobicyclo[2.2.2]octyl, 2-azabicyclo[2.2.2]octyl, 3-azabicyclo[3,2.2]nonyl, 2H-pyrrolyl, 4,4-spiro[2,3-dihydrobenzothiophen-3,3-yl]piperidinyl, 4,4-spiro[indoli-3,3-yl]piperidinyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

“Halogen” includes fluorine, chlorine, bromine and iodine.

Compounds of Formula I contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixtures thereof are encompassed with compounds of Formula I.

Compounds of the Formula I may be separated into diastereoisomeric pairs of enantiomers by, for example, fractional crystallization from a suitable solvent, for example MeOH or ethyl acetate or a mixture thereof. The pair of enantiomers thus obtained may be separated into individual stereoisomers by conventional means, for example by the use of an optically active amine as a resolving agent or on a chiral HPLC column.

Alternatively, any enantiomer of a compound of the general Formula I may be obtained by stereospecific synthesis using optically pure starting materials or reagents of known configuration.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium,. zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine; purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

Compounds of this invention are antagonists of the MCH-1R receptor and as such are useful for the prevention and treatment of disorders or diseases associated with the MCH-1R receptor. Accordingly, another aspect of the present invention provides a method for the treatment (including prevention, alleviation, amelioration or suppression) of diseases or disorders or symptoms mediated by MCH-1R receptor binding and subsequent cell activation, which comprises administering to a mammal an effective amount of a compound of Formula I. Such diseases, disorders, conditions or symptoms are, for example, obesity, diabetes, appetite and eating disorders, cardiovascular disease, hypertension, dyslipidemia, myocardial infarction, gall stones, osteoarthritis, certain cancers, AIDS wasting, cachexia, frailty (particularly in elderly), binge eating disorders including bulimina, anorexia, mental disorders including manic depression, depression, schizophrenia, mood disorders, delirium, dementia, severe mental retardation, anxiety, stress, cognitive disorders, sexual function, reproductive function, kidney function, diuresis, locomotor disorders, attention deficit disorder (ADD), substance abuse disorders and dyskinesias including Parkinson's disease, Parkinson-like syndromes, Tourette's syndrome, Huntington's disease, epilepsy, improving memory function, and spinal muscular atrophy.

The utilities of the present compounds in these diseases or disorders may be demonstrated in animal disease models that have been reported in the literature. The following are examples of such animal disease models: a) suppression of food intake and resultant weight loss in rats (Life Sciences 1998, 63, 113-117); b) reduction of sweet food intake in marmosets (Behavioural Pharm. 1998, 9, 179-181); c) reduction of sucrose and ethanol intake in mice (Psychopharm. 1997, 132, 104-106); d) increased motor activity and place conditioning in rats (Psychopharm. 1998, 135, 324-332; Psychopharmacol. 2000, 151: 25-30); e) spontaneous locomotor activity in mice (J. Pharm. Exp. Ther. 1996, 277, 586-594).

The magnitude of prophylactic or therapeutic dose of a compound of Formula I will, of course, vary with the nature of the severity of the condition to be treated and with the particular compound of Formula I and its route of administration. It will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.001 mg to about 25 mg (preferably from 0.01 mg to about 1 mg) of a compound of Formula I per kg of body weight per day and for cytoprotective use from about 0.1 mg to about 100 mg (preferably from about 1 mg to about 100 mg and more preferably from about 1 mg to about 10 mg) of a compound of Formula I per kg of body weight per day.

In the case where an oral composition is employed, a suitable dosage range is, e.g. from about 0.01 mg to about 100 mg of a compound of Formula I per day, preferably from about 0.1 mg to about 10 mg per day. For oral administration, the compositions are preferably provided in the form of tablets containing from 0.01 to 1,000 mg, preferably 0.01, 0.05, 0.1, 0.5, 1.0, 2.5, 5.0, 10.0, 15.0, 20.0, 25.0, 30.0, 40.0, 50.0 or 1000.0 milligrams of the active ingredient for the symptomatic adjustment of the dosage to the patient to be treated.

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form: of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet maybe prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

The following are examples of representative pharmaceutical dosage forms for the compounds of Formula I:

| Injectable Suspension (I.M.) | mg/mL |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |

Water for injection to a total volume of 1 mL

| Tablet | mg/tablet | |
|---|---|---|
| Compound of Formula I | 25 | |
| Microcrystalline Cellulose | 415 | |
| Povidone | 14.0 | |
| Pregelatinized Starch | 43.5 | |
| Magnesium Stearate | 2.5 | |
| | 500 | |
| Capsule | mg/capsule | |
| Compound of Formula I | 25 | |
| Lactose Powder | 573.5 | |
| Magnesium Stearate | 1.5 | |
| | 600 | |
| Aerosol | Per canister | |
| Compound of Formula I | 24 | mg |
| Lecithin, NF Liq. Conc. | 1.2 | mg |
| Trichlorofluoromethane, NF | 4.025 | g |
| Dichlorodifluoromethane, NF | 12.15 | g |

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in; addition to the compound of Formula I is preferred. Accordingly, the pharmaceutical compositions of the present invention include those that also contain one or more other active ingredients, in addition to a compound of Formula I.

It will be appreciated that for the treatment or prevention of eating disorders, including obesity, bulimia nervosa and compulsive eating disorders, a compound of the present invention may be used in conjunction with other anorectic agents.

The present invention also provides a method for the treatment or prevention of eating disorders, which method comprises administration to a patient in need of such treatment an amount of a compound of the present invention and an amount of an anorectic agent, such that together they give effective relief.

Suitable anorectic agents of use in combination with a compound of the present invention include, but are not limited to, aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolarnine, picilorex and sibutramine; and pharmaceutically acceptable salts thereof.

A particularly suitable class of anorectic agent are the halogenated amphetamine derivatives, including chlorphentermine, cloforex, clortermine, dexfenfluramine, fenfluramine, picilorex and sibutramine; and pharmaceutically acceptble salts thereof.

Particularly preferred halogenated amphetamine derivatives of use in combination with a compound of the present invention include: fenfluramine and dexfenfluramine, and pharmaceutically acceptable salts thereof.

It will be appreciated that for the treatment or prevention of obesity, the compounds of the present invention may also be used in combination with a selective serotonin reuptake inhibitor (SSRI).

The present invention also provides a method for the treatment or prevention of obesity, which method comprises administration to a patient in need of such treatment an amount of a compound of the present invention and an amount of an SSRI, such that together they give effective relief.

Suitable selective serotonin reuptake inhibitors of use in combination with a compound of the present invention include: fluoxetine, fluvoxamine, paroxetine and sertraline, and pharmaceutically acceptable salts thereof.

The present invention also provides a method for the treatment or prevention of obesity, which method comprises administration to a patient in need of such treatment an amount of a compound of the present invention and an amount of growth hormone secretagogues such as those disclosed and specifically described in U.S. Pat. No. 5,536,716; melanocortin agonists such as Melanotan II; , β-3 agonists such as those disclosed and specifically described in patent publications WO94/18161, WO95/29159, WO97/46556, WO98/04526 and WO98/32753; 5Hr-2 agonists; orexin antagonists; melanin concentrating hormone antagonists; galanin antagonists; CCK agonists; GLP-1 agonists; corticotropin-releasing hormone agonists; NPY-5 antagonists; CB1 modulators, such as N-(1-piperidinyl)-5-(4-chlorophenyl)-1-(2,4-dichlorophenyl)-4-methylpyrazole-3-carboxamide (SR141716A), and those described in U.S. Pat. No. 5,624,941 and U.S. Pat No. 6,028,084, PCT Application Nos. WO98/43636, WO98/31227, WO98/41519, WO98/37061, WO00/10967, WO00/10968, WO97/29079, WO99/02499 and WO98/43635, and EPO Application No. EP-658546; and Y1 antagonists, such that together they give effective relief.

As used herein “obesity” refers to a condition whereby a mammal has a Body Mass Index (BM), which is calculated as weight per height squared (kg/m²), of at least 25.9. Conventionally, those persons with normal weight, have a BMI of 19.9 to less than 25.9.

It will be appreciated that for the treatment or prevention of obesity, the compounds of the present invention may also be used in combination with histamine receptor-3 (H3) modulators, CB1 cannabinoid receptor antagonists or inverse agonists, and/or phosphodiesterase-3B (PDE3B) inhibitors.

The obesity described herein may be due to any cause, whether genetic or environmental. Examples of disorders that may result in obesity or be the cause of obesity include overeating and bulimia, polycystic ovarian disease, craniopharyngioma, the Prader-Willi Syndrome, Frohlich's syndrome, Type II diabetes, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia.

“Treatment” (of obesity) refers to reducing the BMI of the mammal to less than about 25.9, and maintaining that weight for at least 6 months. The treatment suitably results in a reduction in food or calorie intake by the mammal.

“Prevention” (of obesity) refers to preventing obesity from occurring if the treatment is administered prior to the onset of the obese condition. Moreover, if treatment is commenced in already obese subjects, such treatment is expected to prevent, or to prevent the progression of, the medical sequelae of obesity, such as, e.g., arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

Excessive weight is a contributing factor to different diseases including hypertension, diabetes, dyslipidemias, cardiovascular disease, gall stones, osteoarthritis and certain forms of cancers. Bringing about a weight loss can be used, for example, to reduce the likelihood of such diseases and as part of a treatment for such diseases. Weight reduction can be achieved by antagonizing MCH-1R receptor activity to obtain, for example, one or more of the following effects: reducing appetite, increasing metabolic rate, reducing fat intake or reducing carbohydrate craving.

Other compounds that may be combined with a compound of Formula I, either administered separately or in the same pharmaceutical compositions, for the treatment of diabetes and other sequelae of excessive weight include, but are not limited to:
(a) insulin sensitizers including (i) PPARγ agonists such as the glitazones (e.g. troglitazone, pioglitazone, englitazone, MCC-555, BRL49653 and the like), and compounds disclosed in WO97/27857, 97/28115, 97/28137 and 97/27847; (ii) biguanides such as metformin and phenformin;
(b) insulin or insulin mimetics;
(c) sulfonylureas, such as tolbutamide and glipizide;
(d) α-glucosidase inhibitors (such as acarbose),
(e) cholesterol lowering agents such as (i) HMG-CoA reductase inhibitors (lovastatin, simvastatin, pravastatin, fluvastatin, atorvastatin, and other statins), (ii) sequestrants (cholestyramine, colestipol and a dialkylaminoalkyl derivatives of a cross-linked dextran), (ii) nicotinyl alcohol nicotinic acid or a salt thereof, (iii) proliferator-activater receptor α agonists such as fenofibric acid derivatives (gemfibrozil, clofibrate, fenofibrate and benzafibrate), (iv) inhibitors of cholesterol absorption for example beta-sitosterol and (acyl CoA:cholesterol acyltransferase) inhibitors for example melinamide, (v) probucol, (vi) vitamin E, and (vii) thyromimetics;
(f) PPARδ agonists, such as those disclosed in WO97/28149;
(g) antiobesity compounds, such as fenfluramine, dexfenfluramine, phentermine, sibutramine, orlistat, or β₃ adrenergic receptor agonists;
(h) feeding behavior modifying agents, such as neuropeptide Y antagonists (e.g. neuropeptide Y5) such as those disclosed in WO 97/19682, WO 97/20820, WO 97/20821, WO 97/20822 and WO 97/20823;
(i) PPARα agonists such as described in WO 97/36579 by Glaxo;
(j) PPARγ antagonists as described in WO97/10813;
(k) serotonin reuptake inhibitors such as fluoxetine and sertraline;
(l) growth hormone secretagogues such as MK-0677.

It will be appreciated that for the treatment or prevention of stress, a compound of the present invention may be used in conjunction with other anti-stress agents, such as anti-anxiety agents. Suitable classes of anti-anxiety agents include benzodiazepines and 5-HT_{1A} agonists or antagonists, especially 5-HT_{1A} partial agonists, and corticotropin releasing factor (CRF) antagonists.

Suitable benzodiazepines include: alprazolam, chlordiazepoxide, clonazepam, chlorazepate, diazepam, halazepam, lorazepam, oxazepam and prazepam, and pharmaceutically acceptable salts thereof.

Suitable 5-HT_{1A} receptor agonists or antagonists include, in particular, the 5-HT_{1A} receptor partial agonists buspirone, flesinoxan, gepirone and ipsapirone, and pharmaceutically acceptable salts thereof.

Suitable CRF antagonists include the 4-tetrahydropyridylpyriridine derivatives disclosed in U.S. Pat. No. 6,187,781; the aryloxy and arylthio-fused pyridine and pyrimidine derivatives disclosed in U.S. Pat. No. 6,124,300; the arylaminofused pyrimidine derivatives disclosed in U.S. Pat. No. 6,107,300; the pyrazole and pyrazolopyrimidine derivatives disclosed in U.S. Pat. No. 5,705,646, U.S. Pat. No. 5,712,303, U.S. Pat. No. 5,968,944, U.S. Pat. No. 5,958,948, U.S. Pat. No. 6,103,900 and U.S. Pat. No. 6,005,109; the tetrahydropteridine derivatives disclosed in U.S. Pat. No. 6,083,948; the benzoperimidine carboxylic acid derivatives disclosed in U.S. Pat. No. 5,861,398; the substituted 4-phenylaminothiazol derivatives disclosed in U.S. Pat. No. 5,880,135; the cyclic CRF analogs disclosed in U.S. Pat. No. 5,493,006, U.S. Pat. No. 5,663,292 and U.S. Pat. No. 5,874,227; and the compounds disclosed in U.S. Pat. No. 5,063,245, U.S. Pat. No. 5,245,009, U.S. Pat. No. 5,510,458 and U.S. Pat. No. 5,109,111; as well as compounds described in International Patent Specification Nos. WO 94/13643, WO 94/13644, WO 94/13661, WO 94/13676 and WO 94/13677.

As used herein, the term “substance abuse disorders” includes substance dependence or abuse with or without physiological dependence. The substances associated with these disorders are: alcohol, amphetamines (or amphetamine-like substances),. caffeine, cannabis, cocaine, hallucinogens, inhalants, nicotine, opioids, phencyclidine (or phencyclidine-like compounds), sedative-hypnotics or benzodiazepines, and other (or unknown) substances and combinations of all of the above.

In particular, the term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances. It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

Similarly, compound of Formula I, will be useful as a partial or complete substitute for conventional pain relievers in preparations wherein they are presently co-administered with other agents or ingredients. Thus in further aspects, the invention encompasses pharmaceutical compositions for modulating the perception of pain comprising a non-toxic therapeutically effective amount of the compound of Formula I as defined above and one or more ingredients such as another pain reliever including acetaminophen or phenacetin, or a cyclooxygenase-2 (COX-2) inhibitor; a potentiator including caffeine; a prostaglandin including misoprostol, enprostil, rioprostil, ornoprostol or rosaprostol: a diuretic; a sedating or non-sedating antihistamine. Examples of cyclooxygenase-2 selective inhibitors include rofecoxib (VIOXX®, see U.S. Pat. No. 5,474,995), etoricoxib (ARCOXIA™ see U.S. Pat. No. 5,861,419), celecoxib (CELEBREX®, see U.S. Pat. No. 5,466,823), valdecoxib (see U.S. Pat. No. 6,633,272), parecoxib (see U.S. Pat. No. 5,932,598), COX-189 (Novartis), BMS347070 (Bristol Myers Squibb), tiracoxib (JTE522, Japan Tobacco), ABT963 (Abbott), CS502 (Sankyo) and GW406381 (GlaxoSmithKline). Other examples of cyclooxygenase-2 inhibitors compounds are disclosed in U.S. Pat. No. 6,020,343. In addition the invention encompasses a method of treating pain comprising: administration to a patient in need of such treatment a non-toxic therapeutically effective amount of the compound of Formula I, optionally co-administered with one or more of such ingredients as listed immediately above.

“Male sexual dysfunction” includes impotence, loss of libido, and erectile dysfunction. “Erectile dysfunction” is a disorder involving the failure of a male mammal to achieve erection, ejaculation, or both. Symptoms of erectile dysfunction include an inability to achieve or maintain an erection, ejaculatory failure, premature ejaculation, or inability to achieve an orgasm. An increase in erectile dysfunction and sexual dysfunction can have numerous underlying causes, including but not limited to (1) aging, (b) an underlying physical dysfunction, such as trauma, surgery, and peripheral vascular disease, and (3) side-effects resulting from drug treatment, depression, and other CNS disorders. “Female sexual dysfunction” can be seen as resulting from multiple components including dysfunction in desire, sexual arousal, sexual receptivity, and orgasm related to disturbances in the clitoris, vagina, periurethral glans, and other trigger points of sexual function. In particular, anatomic and functional modification of such trigger points may diminish the orgasmic potential in breast cancer and gynecologic cancer patients. Treatment of female sexual dysfunction with an MC-4 receptor agonist can result in improved blood flow, improved lubrication, improved sensation, facilitation of reaching orgasm, reduction in the refractory period between orgasms, and improvements in arousal and desire. In a broader sense, “female sexual dysfunction” also incorporates sexual pain, premature labor, and dysmenorrhea.

For the treatment of male and female sexual dysfunction, the compounds of the present invention may be employed in combination with a compound selected from a type V cyclic-GMP-specific phosphodiesterase (PDE-V) inhibitor, such as sildenafil and IC-351 or a pharmaceutically acceptable salt thereof; an alpha-adrenergic receptor antagonist, such as phentolamine and yohimbine or a pharmaceutically acceptable salt thereof; or a dopamine receptor agonist, such as apomorphine or a pharmaceutically acceptable salt thereof.

Suitable antipsychotic agents of use in combination with a compound of the present invention for the treatment of schizophrenia include the phenothiazine, thioxanthene, heterocyclic dibenzazepine, butyrophenone, diphenylbutylpiperidine and indolone classes of antipsychotic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. Suitable examples of dibenzazepines include clozapine and olanzapine. An example of a butyrophenone is haloperidol. An example of a diphenylbutylpiperidine is pimozide. An example of an indolone is molindolone. Other antipsychotic agents include loxapine, sulpiride and risperidone. It will be appreciated that the antipsychotic agents when used in combination with a CB1 receptor modulator may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, olanzapine, haloperidol, pimozide and risperidone are commonly used in a non-salt form.

Other classes of antipsychotic agent of use in combination with a compound of the present invention include dopamine receptor antagonists, especially D2, D3 and D4 dopamine receptor antagonists, and muscarinic M1 receptor agonists. An example of a D3 dopamine receptor antagonist is the compound PNU-99194A. An example of a D4 dopamine receptor antagonist is PNU-101387. An example of a muscarinic M1 receptor agonist is xanomeline.

Another class of antipsychotic agent of use in combination with a CB1 receptor modulator is the 5-HT^{2A} receptor antagonists, examples of which include MDL100907 and fananserin. Also of use in combination with a compound of the present invention are the serotonin dopamine antagonists (SDAs) which are believed to combine 5-HT_{2A} and dopamine receptor antagonist activity, examples of which include olanzapine and ziperasidone.

It will be appreciated that for the treatment of depression or anxiety, a compound of the present invention may be used in conjunction with other anti-depressant or anti-anxiety agents.

Suitable classes of anti-depressant agents include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, cc-adrenoreceptor antagonists, neurokinin-1 receptor antagonists and atypical anti-depressants.

Suitable norepinephrine reuptake inhibitors include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Suitable selective serotonin reuptake inhibitors include those described supra.

Suitable monoamine oxidase inhibitors include: isocarboxazid, phenelzine, tranylcypromine and selegiline, and pharmaceutically acceptable salts thereof.

Suitable reversible inhibitors of monoamine. oxidase include: moclobemide, and pharmaceutically acceptable salts thereof.

Suitable serotonin and noradrenaline reuptake inhibitors of use in the present invention include: venlafaxine, and pharmaceutically acceptable salts thereof.

Suitable CRF antagonists include those compounds described hereinabove.

Suitable atypical anti-depressants include: bupropion, lithium, nefazodone, trazodone and viloxazine, and pharmaceutically acceptable salts thereof.

Suitable classes of anti-anxiety agents include benzodiazepines and 5-HT_{1A} agonists or antagonists, especially 5-HT_{1A} partial. agonists, and corticotropin releasing factor (CRF) antagonists.

The neurokinin-1 receptor antagonist may be peptidal or non-peptidal in nature, however, the use of a non-peptidal neurokinin-1 receptor antagonist is preferred. In a preferred embodiment, the neurokinin-1 receptor antagonist is a CNS-penetrant neurokinin-1 receptor antagonist. In addition, for convenience the use of an orally active neurokinin-1 receptor antagonist is preferred. To facilitate dosing, it is also preferred that the neurokinin-1 receptor antagonist is a long acting neurokinin-1 receptor antagonist. An especially preferred class of neurokinin-1 receptor antagonists of use in the present invention are those compounds which are orally active and long acting.

Neurokinin-1 receptor antagonists of use in the present invention are fully described, for example, in U.S. Pat. Nos. 5,162,339, 5,232,929, 5,242,930, 5,373,003, 5,387,595, 5,459,270, 5,494,926, 5,496,833, 5,637,699; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006,0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, and 97/49710; and in British Patent Publication Nos. 2 266 529, 2 268 931, 2 269 170, 2 269 590, 2 271 774, 2 292 144, 2 293 168, 2 293 169, and 2 302 689.

Specific neurokinin-1 receptor antagonists of use in the present invention include:
(±)-(2R3R,2S3S)—N-{[2-cyclopropoxy-5-(trifluoromethoxy)-phenyl]methyl}-2-phenylpiperidin-3-arnine;
2-(S)-(3,5-bis(trifluoromethyl)benzyloxy)-3(S)-(4-fluorophenyl)-4-(3-(5-oxo-1H,4H-1,2,4-triazolo)methyl)morpholine;
2-(R)-(1-(R)-(3,5-bis(trifluoromethyl)phenyl)ethoxy)-4-(3-(5-oxo-1H,4H-1,2,4-triazolo)methyl)-3-(S)-phenyl-morpholine;
2-(S)-(3,5-bis(trifluoromethyl)benzyloxy)-4-(3-(5-oxo-1H,4H-1,2,4-triazolo)methyl)-3-(S)-phenyl-morpholine;
2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine;
2-(R)-(1-(R)-(3,5-bis(trifluoromethyl)phenyl)ethoxy)-4-(5-(N,N-dimethylamino)methyl-1,2,3-triazol-4-yl)methyl-3-(S)-phenylmorpholine;
2-(R)-(1-(R)-(3,5-bis(trifluoromethyl)phenyl)ethoxy)4-(5-(N,N-dimethylamino)methyl-1,2,3-triazol-4-yl)methyl-3-(S)-(4-fluorophenyl)morpholine;
(3S ,5R,6S)-3-[2-cyclopropoxy-5-(trifluoromethoxy)phenyl]-6-phenyl-1-oxa-7-aza-spiro[4.5]decane;
(3R,5R,6S)-3-[2-cyclopropoxy-5-(trifluoromethoxy)phenyl]-6-phenyl-1-oxa-7-aza-spiro[4.5]decane;
2-(R)-(1-(S)-(3,5-bis(trifluoromethyl)phenyl)-2-hydroxyethoxy)-3-(S)-(4-fluorophenyl)-4-(1,2,4-triazol-3-yl)methylmorpholine;
2-(R)-(1-(R)-(3,5-bis(trifluoromethyl)phenyl)ethoxy)-3-(S)-(4-fluorophenyl)-4-(3-(4-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(1-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(2-monophosphoryl-5-oxo-1H-1,2,4-triazolo)methyl)morpholine;
2-(R)-(1-(R)-(3,5-bis(trifluoromethyl)phenyl)ethoxy)-3-(S)-(4-fluorophenyl)-4-(3-(5-oxyphosphoryl-1H-1,2,4-triazolo)methyl)morpholine;
2-(S)-(1-(R)-(3,5-bis(trifluoromethyl)phenyl)ethoxy)-3-(S)-(4-fluorophenyl)-4-(3-(1-monophosphoryl-5-oxo-4H-1,2,4-triazolo)methyl)morpholine;
2-(R)-(1-(R)-(3,5-bis(trifluoromethyl)phenyl)ethoxy)-4-(4-N,N-dimethylaminobut-2-yn-yl)-3-(S)-(4-fluorophenyl)morpholine;
or a pharmaceutically acceptable salt thereof.

Suitable benzodiazepines include those described previously herein.

Suitable 5-HT_{1A} receptor agonists or antagonists include, in particular, those described supra.

For the treatment of autism, the compounds of the present invention may be used in combination with butyrophenones.

For the treatment of Parkinson's disease and Parkinson-like syndromes, the compounds of the present invention may be used in combination with levodopa, carbidopa/levodopa, amantadine, bromocryptine and other ergot alkaloids, anticholinergic medications such as benztropine, trihexyphenidyl, antihistamines such as diphenhydramine and orphenadrine, mild sedatives, tricyclic antidepressants such as amitriptiline and others described supra, and propanolol.

For the treatment of Huntingdon's Chorea, the compounds of the present invention may be used in combination with phenothiazine, chlorpromazine, and butyrophenone neuroleptics such as haloperidol or reserpine.

For the treatment of epilepsy, the compounds of the present invention may be used together with anticonvulsants such as penytoin, phenobarbital, primidone, carbamazepine, trimethadione, clonazepam, valproate and ethosuximide

MCH-1R antagonist compounds can be provided in kit. Such a kit typically contains an active compound in dosage forms for administration. A dosage form contains a sufficient amount of active compound such that a beneficial effect can be obtained when administered to a patient during regular intervals, such as 1 to 6 times a day, during the course of 1 or more days. Preferably, a kit contains instructions indicating the use of the dosage form for weight reduction (e.g., to treat obesity or overweight) or stress reduction, and the amount of dosage form to be taken over a specified time period.

The method of treatment of this invention comprises a method of treating melanin concentrating hormone receptor mediated diseases by administering to a patient in need of such treatment a non-toxic therapeutically effective amount of a compound of this invention that selectively antagonizes the MCH-1R receptor in preference to the other G-protein coupled receptors. In particular, the present invention comprises a method of treating MCR-1R receptor subtype mediated diseases by administering to a patient in need of such treatment a non-toxic therapeutically effective amount of a compound of this invention that selectively antagonizes the MCH-1R receptor.

The weight ratio of the compound of the Formula I to the second active ingredient may be varied and will depend upon the effective dose of each ingredient. Generally, an effective dose of each will be used. Thus, for example, when a compound of the Formula I is combined with a β-3 agonist the weight ratio of the compound of the Formula I to the β-3 agonist will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

The compounds of Formula I of the present invention can be prepared according to the procedures of the following Schemes and Examples, using appropriate materials and are further exemplified by the following specific examples. Moreover, by utilizing the procedures described with the disclosure contained herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously hereinabove. The free amine bases corresponding to the isolated salts can be generated by neutralization with a suitable base, such as aqueous sodium hydrogencarbonate, sodium carbonate, sodium hydroxide, and potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation, or crystallization. All temperatures are degrees Celsius unless otherwise noted. Mass spectra (MS) were measured by electron-spray ionization.

The phrase “standard peptide coupling reaction conditions” means coupling a carboxylic acid with an amine using an acid activating agent such as 1-(3-dimethylamninopropyl)-3-ethylcarbodiimide HCl (EDC), 1,3-dicyclohexylcarbodiimnide DCC), and benzotriazol-1-yloxytris(dimethylamino)phosphonium hexafluorophosphate (BOP) in an inert solvent such as dichloromethane in the presence of a catalyst such as 4-dimethylaminopyridine (DMAP) or 1-hydroxybenzotriazole hydrate (HOBT). The use of protecting groups for the amine, carboxylic acid or other functionalities to facilitate the desired reaction and minimize undesired reactions is well documented. Conditions required to remove protecting groups are found in standard textbooks such as Greene, T. and Wuts, P. G. M., Protective Groups in Organic Synthesis, John Wiley & Sons, Inc., New York, N.Y., 1991. Benzyloxycarbonyl (CBZ) and t-butyloxycarbonyl (BOC) protecting groups are commonly used protecting groups in organic synthesis, and conditions for their removal are known to those skilled in the art. For example, CBZ may be removed by catalytic hydrogenation in the presence of a noble metal or its oxide such as palladium on activated carbon in a protic solvent such as methanol or ethanol. In cases where catalytic hydrogenation is contraindicated due to the presence of other potentially reactive functionalities, removal of CBZ groups can also be achieved by treatment with a solution of hydrogen bromide in acetic acid or by treatment with a mixture of trifluoroacetic acid (TFA) and dimethylsulfide. Removal of BOC protecting groups is carried out with a strong acid, such as trifluoroacetic acid, hydrochloric acid, or hydrogen chloride gas, in a solvent such as methylene chloride, methanol, or ethyl acetate.

Abbreviations Used in the Description of the Preparation of the Compounds of the Present Invention and Biological Assays:

BOC (boc) t-butyloxycarbonyl
BOP benzotriazol-1-yloxytris(dimethylamino)phosphonium hexafluorophosphate
BSA Bovine serum albumin
Bu butyl
calc. calculated
CBZ (Cbz) benzyloxycarbonyl
c-hex cyclohexyl
c-pen cyclopentyl
c-pro cyclopropyl
DCC 1,3-dicyclohexylcarbodiimide
DIEA diisopropylethylamine
DMAP 4-dimethylaminopyridine
DMF N,N-dimethylformamide
ECB buffer Extra-cellular buffer: 140 nM NaCl, 20 nM KCl, 20 mM HEPES-NaOH pH 7.4, 5 mM glucose, 1 mM MgCl₂, 1 mM CaCl₂, 0.1 mg/mL BSA
EDC 1-(3-dimethylaminopropyl)-3-ethylcarbodiimide HCl
EDTA Ethylenediamine tetraacetic acid
eq. equivalent(s)
ES-MS electron spray ion-mass spectroscopy
Et ethyl
EtOAc ethyl acetate
h hour
HEPES 4-(2-hydroxyethyl)piperazine-1-ethane sulfonic acid
HOAc acetic acid
HOBt 1-hydroxybenzotriazole hydrate
HPLC high performance liquid chromatography
Me methyl
MF molecular formula
MS mass spectrum
Ms methanesulfonyl
POCl₃ Phosphorous oxychloride
Ph phenyl
Pr propyl
prep. prepared
r.t. room temperature
TEA triethylamine
TFA trifluoroacetic acid
THF tetrahydrofuran
TLC thin-layer chromatography.
   General preparation of 4-amino-6-substituted quinoline intermediates 7 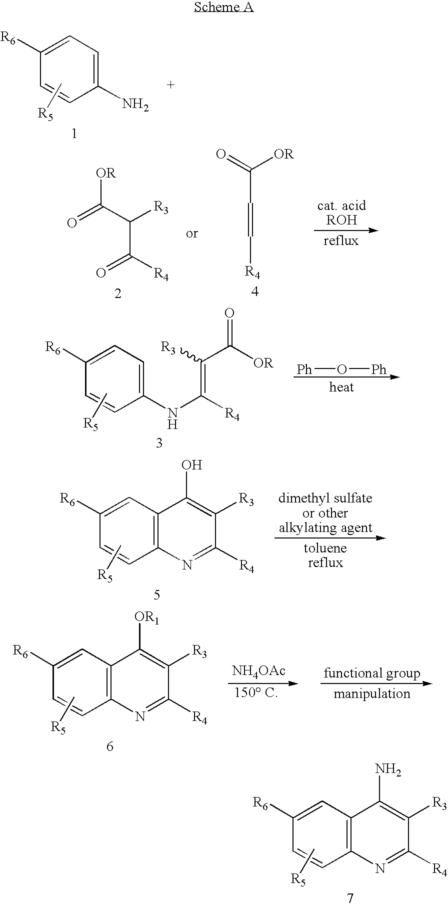
   There are many known preparation of quinolines available to those skilled in the art. Scheme A illustrates the preparation of substituted quinolines utilized for the present invention and follows closely to published procedures reported by Lanza et al. J. Med. Chem. 1992, 35, 252-258. Heating of substituted anilines 1, in particular, 4-substituted anilines, with a variety of substituted ketoesters 2 with an acid catalyst such as hydrochloric or p-toluenesulfonic acid in an appropriate solvent for several hours affords 3-(substituted phenyl) ester intermediates 3. Isolation of these intermediates 3 or simply further heating crude intermediates 3 at higher temperature in an inert solvent such as diphenyl ether provides substituted 4-hydroxyquinoline intermediates 5. Alternatively, heating aniline starting materials 1 and alkynyl ester intermediates 4 with an acid catalyst provides the intermediates 3 which may be converted in like fashion (with or without isolation) by further heating to quinoline intermediates 5. Alkylation of the 4-hydroxyl group of intermediates 5 under a variety of conditions such as treatment of the 4-hydroxyquinoline intermediates 5 with dimethylsulfate or similar alkylating agents in toluene under reflux affords 4-alkoxyquinoline intermediates 6. Further substitution of the 4-position occurs by heating 4-alkoxyquinoline intermediates 6 (R₁=Me) with an ammonium salt such as ammonium acetate to afford 4-aminoquinoline intermediates 7. Alternatively, heating 4-alkoxyquinoline intermediates 6 (R₁=Me) in a sealed tube with an ammonia solution, substituted amine (neat or in an appropriate solvent) or an amine salt and appropriate base provides 4-aminoquinoline intermediates 7. Standard functional group manipulation of substituents of the quinoline ring system known to those skilled in the art provides compounds 7 of the present invention.
   General preparation of N-substituted 4-aminoquinoline intermediates 9

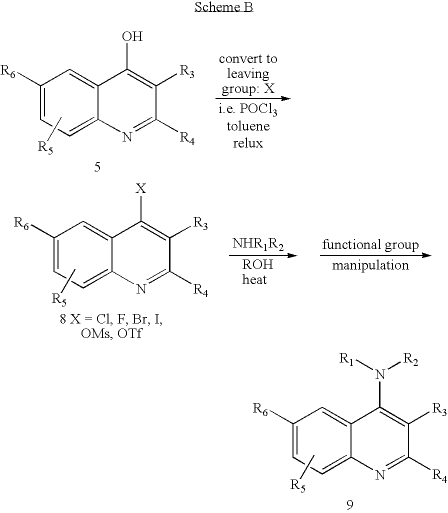

An improved preparation of N-substituted 4-aminoquinoline intermediates 9 is available as described in Scheme B. Substituted 4-hydroxyquinoline intermediates 5 may be converted to 4-chloroquinoline intermediates 8 (X═Cl) by a variety of methods such as treatment with a chlorinating reagent such as phosphorous oxychloride in refluxing toluene. This transformation creates an improved leaving group at the 4-position of the quinoline ring. Similarly, the 4-hydroxyl group of intermediate 5 may be converted by those skilled in the art to other known improved leaving groups, for example, but not limited to, fluoride, bromide, iodide, methanesulfonate or trifluoromethanesulfonate. Heating of the 4-chloroquinolines 8 (X═Cl) or similar quinoline intermediates 8 with a leaving group at the 4-position with ammonia, a primary or secondary amine in an appropriate solvent provides the N-substituted 4-aminoquinoline intermediates 9. Ammonia or volatile amines may be heated neat or with an appropriate solvent in a sealed tube to provide these intermediates. Alternatively, amine salts combined with an appropriate tertiary amine base or inorganic base such as sodium bicarbonate may provide the desired substituted aminoquinoline intermediates 9. Standard functional group manipulation of substituents of the quinoline ring system known to those skilled in the art provides compounds 9 of the present invention.

### General Preparation of 4,6-diaminoquinoline Intermediates

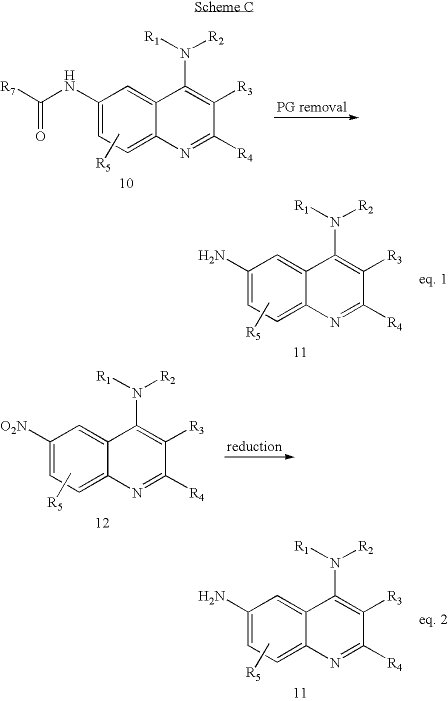 4,6-Diaminoquinoline intermediates 11 may be prepared as described in Scheme C. 4,6-Diaminoquinoline intermediates 10 containing protected 6-amino groups may be converted to the 6-amino derivatives 11 by removal of the protecting groups using methods known to those skilled in the art as described above (eq. 1). Such protecting groups may be carboxamides such as acetyl groups or carbamate protecting groups such as BOC-group or CBZ group, for example. Alternatively 4-amino-6-nitroquinoline intermediates 12 may be converted to 4,6-diaminoquinoline intermediates 11 by reduction of the nitro group using a variety of methods known to those skilled in the art (eq. 2). For example, treatment of the nitro group of intermediates 12 with chemical reducing agents such as tin (IR) chloride, ferric chloride, hydrazine system in the presence of carbon, or lithium aluminium hydride may produce amino groups of intermediates 11. Similarly catalytic reduction of nitro groups of intermediates 12 with hydrogen in the presence of a noble metal catalyst such as palladium on carbon or platinum oxide may provide the desired amino compound 11. Choice of reducing conditions by those skilled in the art may be dictated by other functional groups present in the intermediates 12 which are contraindicated to the nitro group reducing conditions. 6-Nitroquinoline intermediates 12 may be prepared by those skilled in the art from appropriate substituted nitroanilines and other appropriate starting materials using the synthetic route outlined in Schemes A and B.

### General preparation of N-(4-aminoquinolin-6-yl)carboxamides

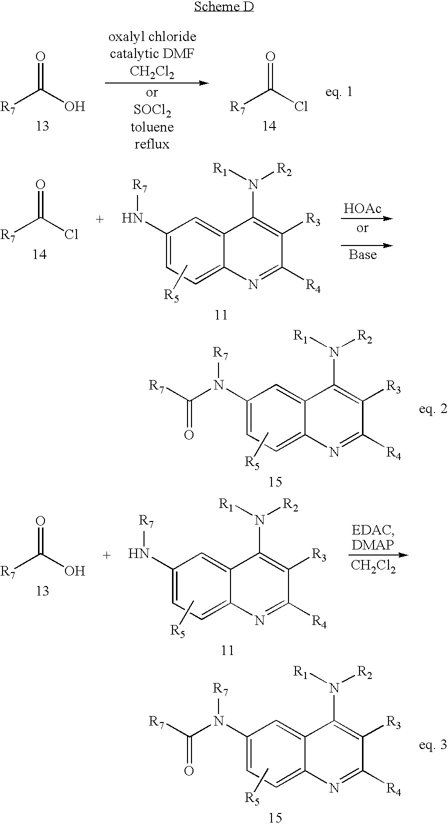

Compounds of the present invention may be prepared by those skilled in the art by reaction of the 4,6-diaminoquinoline intermediates 11 with carboxylic acid derivatives 13 under a variety of conditions to provide the desired N-(4-aminoquinolin-6-yl)carboxamides 15 as described in Scheme D. Treatment of carboxylic acid intermediates 13 with oxalyl chloride with a catalytic amount of N,N-dimethylformamnide (DMF) in an inert solvent such as methylene chloride under an inert atmosphere provides the corresponding acid chloride intermediates 14. Similarly, treatment of the carboxylic acid intermediates 13 with thionyl chloride in toluene at reflux provides acid chloride intermediates 14. Reaction of the 4,6-diaminoquinoline intermediates 11 with the acid chloride intermediates 14 in acetic acid solvent provides the desired N-(4-aminoquinolin-6-yl)carboxamides 15, which may be isolated as salts from the reaction mixture by filtration or other methods known to those skilled in the art. Alternatively, products 15 may be purified by a variety of techniques known to those skilled in the art such as (but not limited to) preparative thin layer chromatography (tlc), HPLC, reverse phase HPLC or column chromatography on a variety of adsorbents such as silica gel or alumina. Similarly, reaction of the 4,6-diaminoquinoline intermediates 11 with acid chloride derivatives 14 in the presence of a tertiary amine or other base in an inert solvent such as methylene chloride affords the desired N-(4-aminoquinolin-6-yl)carboxamides 15. Alternatively, N-(4-aminoquinolin-6-yl)carboxamides 15 may be prepared directly from carboxylic acid derivatives 13 and the 4,6-diaminoquinoline intermediates 11 using a variety of standard peptide coupling reagents as described earlier, such as EDC and DMAP, in an inert solvent such as methylene chloride followed by standard workup and purification as described earlier.

Carboxylic acid intermediates 13 are available from a wide range of commercial sources. Alternatively, carboxylic acid derivatives 13 may be prepared by a variety of methods known to those skilled in the art such as, but not limited to, oxidation of other functional groups, carbonylation, saponification of ester intermediates, or deprotection of protected carboxylic acids. Homologated carboxylic acids may be prepared from carboxylic acids by conversion to the corresponding carboxaldehyde intermediates (or directly from available carboxaldehydes) followed by homologation utilizing stabilized Wittig or Horner-Emmons reagents to provide unsaturated acid or ester intermediates. These intermediates may be converted directly to carboxylic acid derivatives 13. Alternatively, the resulting olefin may be functionalized or reduced to the saturated derivative by a variety of conditions known to those skilled in the art such as by catalytic hydrogenation in the presence of a noble metal catalyst such as palladium on carbon or platinum oxide. These saturated intermediates may in turn be converted to carboxylic acid derivatives 13.

### General Preparation of 4-aminoquinolin-6-carboxamide and Related Derivatives

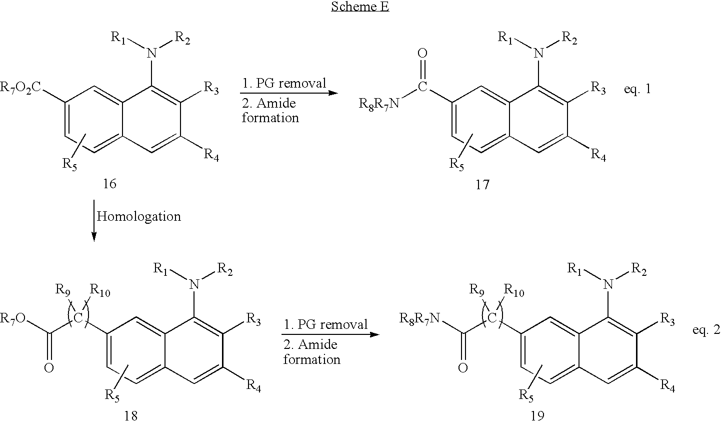

4-Aminoquinolin-6-carboxamide derivatives 17 may be prepared as outlined in Scheme E from 4-amino-6-substituted quinoline derivatives 16 described in Scheme A, wherein the 6-substituent is a carboxylic acid or protected carboxylic acid derivative. Treatment of the carboxylic acid intermediate 16 (R₇═H) directly with an amine under standard peptide coupling conditions such as EDC and DMAP in an inert solvant such as methylene chloride provides the desired quinoline-6-carboxamides 17. Similarly, removal of the protecting group of the carboxylic acid derivative 16 followed by carboxamide formation affords the quinoline-6-carboxamides 17. Homologated analogs may be prepared by homologation of the carboxylic acid intermediates 16 or other intermediates derived thereof using methods known to those skilled in the art such as but not limited to the Arndt-Eistert homologation, or by the sequence of conversion of the acid to the alcohol, leaving group formation, cyanide displacement followed by hydrolysis to the homologated carboxylic acid intermediates 18. Similarly, the carboxylic acid intermediates 16 may be converted to the carboxaldehyde intermediate followed by Wittig or Horner-Emmons homologation and subsequent functional group manipulation as described earlier. Alternatively, homologated carboxylic acid intermediates 18 may be prepared by those skilled in the art from substituted aniline intermediates containing the required homologated acid and other appropriate starting materials using the quinoline synthesis outlined in Schemes A and B. Finally, theses homologated carboxylic acid intermediates 18 may be converted by standard peptide coupling techniques such as those described in Scheme D, with a variety of amines to homologated carboxamide derivatives 19.

### General Preparation of 4-amino-6-heterocycle Substituted quinoline Derivatives and Related Analogs

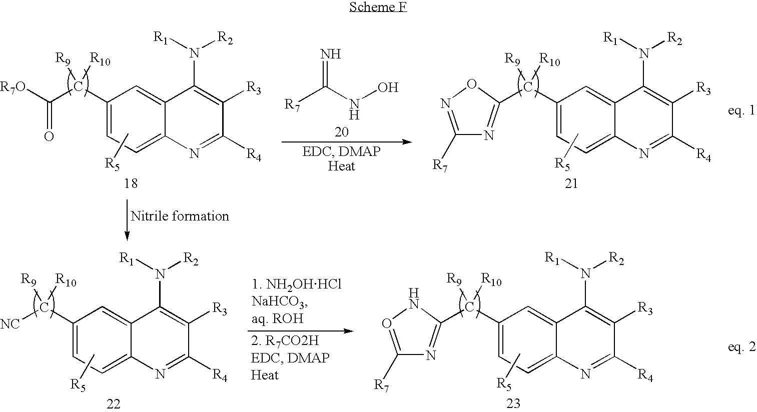

Quinoline derivatives containing heterocycle groups at the 6-position in place of 4-aminoquinoline-6-carboxamide or related analogs or in place of N-(4-aminoquinoline-6-yl)carboxamide or related analogs may be prepared as outlined in Scheme F from quinoline-6-carboxylic acid derivatives 18 or related bomologs. Oxadiazolyl or related heterocyclic derivatives are known to be useful replacements for carboxamide, urea, sulfonamide and other hydrogen bond donating functional groups. Removal of these hydrogen bonding groups may increase water solubility, remove waters of hydration or vary other physical chemical properties that may improve pharmacokinetic parameters such as oral absorption, oral bioavailability or metabolic disposition of these compounds.

These heterocycle substituted quinoline derivatives may be prepared by a variety of methods known to those skilled in the art. For example, treatment of quinolin-6-carboxylic acid intermediates 18 with EDC and DMAP in the presence of an amidoxime derivative 20 followed by heating at reflux in an inert solvent such 1,4-dioxane or 1,2-dimethoxyethane provides (3-substituted-1,2,4-oxadiazol-5yl)quinolin-4-yl amine derivatives 21. Similarly, homologated 4-aminoquinolin-6-yl carboxylic acid intermediates 18 provide the related homologated (3-substituted-1,2,4-oxadiazol-5yl)quinolin-4-yl amine analogs 21. Amidoxime intermediates 20 may be commercially available or may be prepared from nitrile intermediates by treatment with hydroxylamine hydrochloride in the presence of an inorganic base such as sodium bicarbonate in an alcoholic solvent.

Isomeric 6-(5-substituted-1,2,4-oxadiazol-3yl)quinolin-4-amines 23 may be prepared in a similar fashion from 4-aminoquinoline-6-nitrile intermediates 22 or related homologs. 4-Aminoquinoline-6-nitrile intermediates 22 may be prepared as outlined is Scheme A directly from nitrile substituted anilines. Alternatively, quinoline-6-carboxylic acid derivatives 18 may be converted to quinoline-6-carboxamide derivatives as described earlier followed by dehydration using a variety of methods known to those skilled in the art. Reaction of the nitrile intermediates 22 with hydroxylamine as described above affords the corresponding arnidoxime intermediates. Coupling of the amidoxime intermediates with a carboxylic acid derivative 13 in the presence of EDC and DMAP followed by heating in an inert solvent provides the isomeric (5-substituted-1,2,4-oxadiazol-3yl)quinolin-4-amine analogs 23. Similarly, homologated 4-aminoquinolin-6-yl-carboxylic acid intermediates 18 may be converted homologated nitrile intermediates 22 then by analogy to related (5-substituted-1,2,4-oxadiazol-3yl)quinolin-4-amine homologs 23.

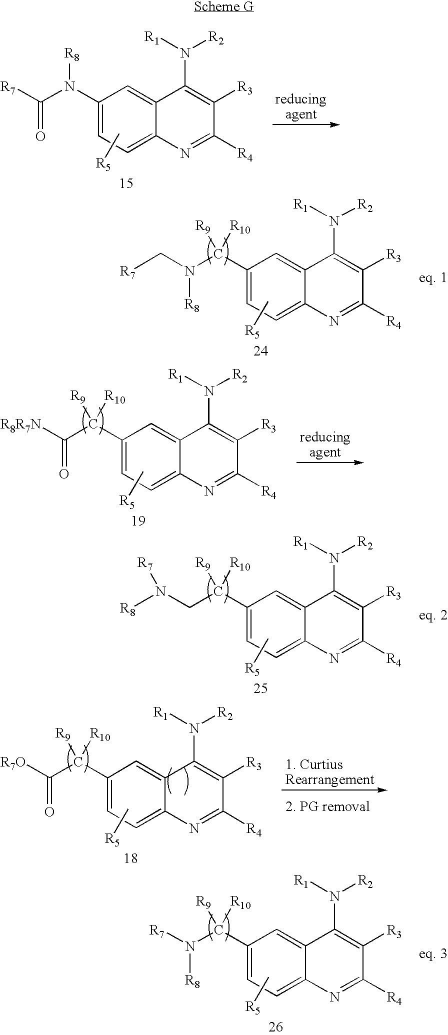

Preparation of further 6-substituted-4,6-diaminoquinoline derivatives is outlined in Scheme G. Simple chemical reduction of the carboxamide group of N-(4-aminoquinolin-6-yl)carboxamide intermediates 15 (eq. 1) and 4-aminoquinolin-6-carboxamide intermediates 19 (eq. 2) by a variety of reducing agents known to those skilled in the art, such as borane derivatives or lithium aluminium hydride, affords the 6-substituted-4,6-diaminoquinoline derivatives 24 and 25 respectively. Alternatively, carboxylic acid intermediates 18 may be converted to amine derivatives 26 by rearrangement reactions such as the Curtius reaction or related rearrangement reactions known to those skilled in the art. Hydrolysis of amine intermediates or removal of protecting groups resulting from the rearrangement reactions may provide the desired 4,6-diaminoquinoline derivatives 26.

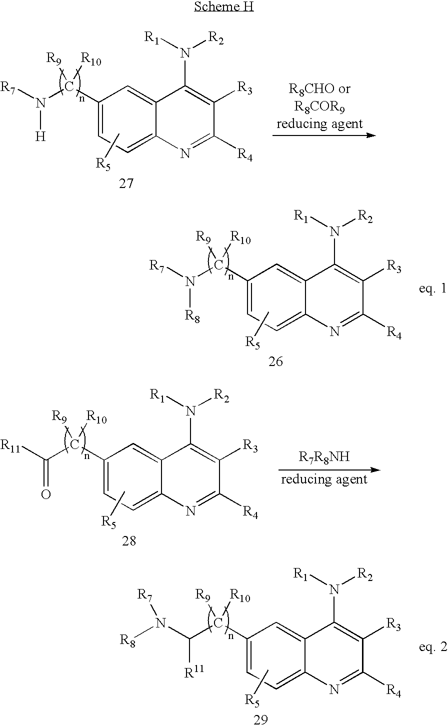

Similarly, other quinolin-4,6-diamine derivatives 27 may be converted to quinolin-4,6-diamine derivatives 26 by reductive amination with a carboxaldehyde or ketone derivative (Scheme H, eq. 1) or by first, carboxamide formation, followed by further reduction of the carboxamide intermediate to the quinolin4,6-diamine derivatives 26. Alternatively, (4-aminoquinolin-6-yl)carboxaldehyde intermediates 28 (R₇═H, eq. 2) or related ketone intermediates (R₇═C, eq. 2) may be converted to quinolin-4,6-diamine derivatives 29 by reductive amination with a variety of amines under a variety of conditions known to those skilled in the art such as sodium cyanoborohydride in the presence of a drying agent and acid buffer in an appropriate solvent such as methanol. (4-Aminoquinolin-6-yl)carboxaldehyde intermediates 28 or related homologated intermediates may be prepared by a variety of methods known to those skilled in the art. For example, oxidation of related alcohol derivatives or reduction of carboxylic acid or related carboxamide ester or nitrile derivatives may provide the desired (4-aminoquinolin-6-yl)carboxaldehyde intermediates 28 or related homologs. Similarly, (4-aminoquinolin-6-yl)ketone intermediates 28 or related homologs may be prepared from above intermediates by many methods known to those skilled in the art. Alternatively, quinoline carboxaldehyde or ketone intermediates 28 may be reduced to the corresponding alcohol intermediates, subsequent leaving group formation then displacement with a suitable amine or surrogate amine nucleophile. Further functional group manipulation or protecting group removal may provide quinolin-4,6-diamine derivatives 29.

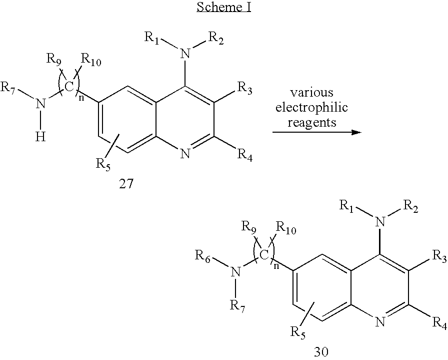

Further derivatives of amine 27 may be prepared by reaction of the amine with a variety of electrophiles such as carboxylic acids or their acid chlorides, isocyanates, carbamoyl chlorides, ketenes, chloroformates, sulfonic acids or their sulfonyl chloride to provide further derivatives of the present invention of the general structure 30 (Scheme I).

The following Examples are provided to illustrate the invention and are not to be construed as limiting the scope of the invention in any manner.

### EXAMPLE 1

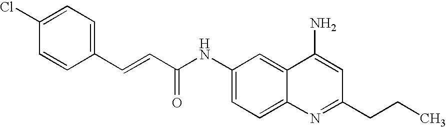

### (2E)-N-(4-Amino-2-propylguinolin-6-yl)-3-(4-chlorophenyl)prop-2-enamide

### Step A: Preparation of ethyl (2E)- and (2Z)-3-{[4-(acetylamino)phenyl]amino}hex-2-enoate

A mixture of N-(4-arninophenyl)acetamnide (9.7 g, 65 mmol), ethyl 3-oxohexanoate (10 g, 65 mmol) and 2 drops conc. HCl in 30 mL ethanol was heated at reflux overnight. After approximately 18 h, the reaction mixture was cooled to r.t. and the solids collected by filtration. The solids were washed with methanol and air dried to afford the crude product as a solid, which was used without further purification in the subsequent reaction.

### Step B: Preparation of N-(4-hydroxy-2-propylguinolin-6-yl)acetamide

The crude product (9.0 g) from Step A was mixed with 50 mL of diphenylether. The mixture was heated with a heating mantle at 260° for 2 h then cooled to r.t. The resulting solid was collected by filtration, washed with EtOAc to give a grey solid, which was used directly in the next step.

### Step C: Preparation of N-(4-methoxy-2-propylquinolin-6-yl)acetamide

The crude product (5.9 g) from Step B and dimethylsulfate (4.6 mL, 48 mmol) were mixed in toluene and heated at reflux for 2.5 h. The reaction mixture was cooled to r.t. and the precipitate was collected by filtration. The solids were washed with toluene, air dried then added to a mixture of 50 mL 1N aq. NaOH and 100 mL EtOAc. The solids were filtered and washed with EtOAc. The filtrate was transferred to a separatory funnel and the layers separated. The aqueous layer was extracted with excess EtOAc. The organic layers were combined and the solvent removed under vacuum to afford the product as a yellow solid, MS: m/z 259 (MH⁺).

### Step D: Preparation of N-(4-amino-2-propylquinolin-6-yl)acetamide

An intimate mixture of the crude product (4.0 g) from Step C and ammonium acetate (40 g, 52 mmol) were heated at 140° to 150° for 4 h. The reaction mixture was cooled to r.t. to provide the crude product which used immediately without further purification.

### Step E: Preparation of 2-propylguinoline-4,6-diamine

To the above crude reaction mixture from Step D was added 30 mL water and 40 mL conc. HCl. The resulting mixture was heated at 90° for 5 h then cooled to r.t. The remaining precipitate was collected by filtration. The aqueous filtrate was concentrated under vacuum then made basic by addition of aq. sodium hydroxide. The aqueous mixture was transferred to a separatory funnel and extracted with excess EtOAc. The organic layers were combined, dried with a drying agent and the solvent removed under vacuum to afford a solid, MS: m/z 202 (MH⁺).

### Step F: Preparation of (2E)-3-(4-chlorophenyl)prop-2-enoyl chloride

To a solution of (2E)-3-(4-chlorophenyl)prop-2-enoic acid (2.0 g, 11 mmol) in 50 mL methylene chloride was added oxalyl chloride (1.05 mL, 12.1 mmol) and N,N-dimethylformamide (0.05 mL, 0.6 mmol). The resulting mixture was stirred at r.t. for 6 h. The solvent was removed under vacuum. The resulting solid was diluted with hexanes and the solvent removed under vacuum to provide an off-white solid, which was used without further purification.

### Step G: Preparation of (2E)-N-(4-Amino-2-propylguinolin-6-yl)-3-(4-chlorophenyl)prop-2-enamide

To a solution of the product of Step E (60 mg, 0.3 mmol) in 1.5 mL HOAc was added the product of Step F (64 mg, 0.32 mmol). The resulting mixture was stirred at r.t. for 6 h then the solvent removed under vacuum. The residue was purified by preparative TLC eluting with chloroform/2N ammonia in methanol (9/1) to afford the product, MS: m/z 366 (MH⁺).

Following a procedure similar to that described above for Example 1, the following compounds were prepared from 2-propylquinoline-4,6-diamine (Example 1, Step E):

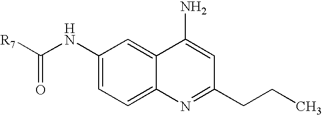

| | | Parent Ion |
|---|---|---|
| Ex. # | R₇ | (MH+) m/z |
| 2 | 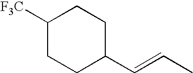 | 406 |
| 3 | 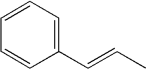 | 332 |
| 4 | 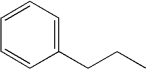 | 334 |
| 5 | 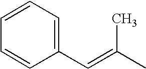 | 346 |
| 6 | 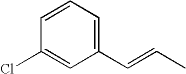 | 366 |
| 7 | 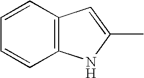 | 345 |
| 8 | 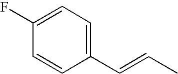 | 350 |
| 9 | 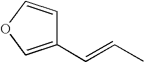 | 322 |
| 10 | 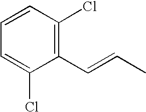 | 400 |
| 11 | 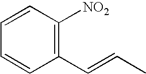 | 377 |
| 12 | 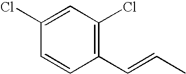 | 400 |
| 13 | 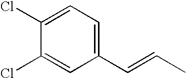 | 400 |
| 14 | 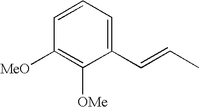 | 392 |
| 15 | 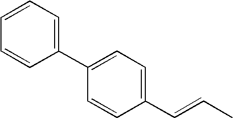 | 408 |
| 16 | 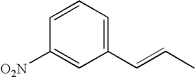 | 377 |
| 17 | 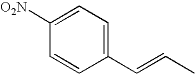 | 377 |
| 18 | 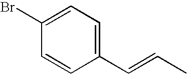 | 412 |
| 19 | 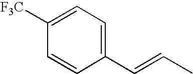 | 400 |
| 20 | 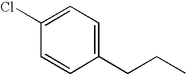 | 346 |
| 21 | 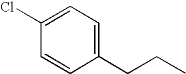 | 368 |
| 22 | 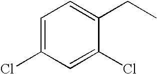 | 388 |
| 23 | 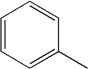 | 306 |
| 24 | 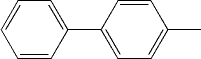 | 382 |
| 25 | 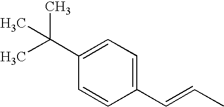 | 388 |
| 26 | 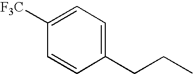 | 402 |
| 27 | 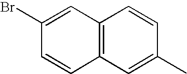 | 434 |
| 28 | 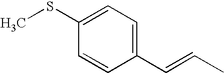 | 378 |
| 29 | 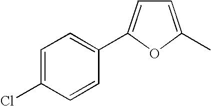 | 406 |
| 30 | 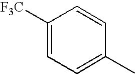 | 374 |
| 31 | 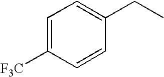 | 388 |
| 32 | 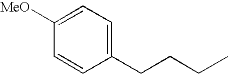 | 378 |
| 33 | 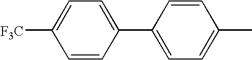 | 450 |
| 34 | 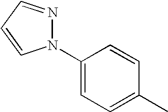 | 372 |
| 35 | 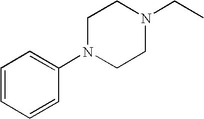 | 404 |
| 36 | 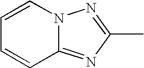 | 348 |
| 37 | 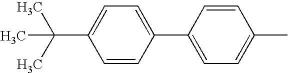 | 438 |
| 38 | 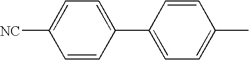 | 407 |
| 39 | 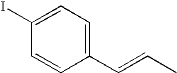 | 458 |
| 40 | 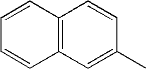 | 356 |
| 41 | 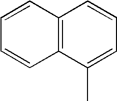 | 356 |
| 42 | 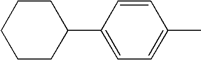 | 388 |
| 43 | 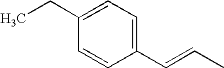 | 360 |
| 44 | 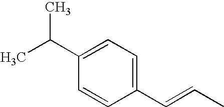 | 374 |
| 45 | 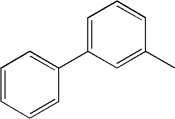 | 382 |
| 46 | 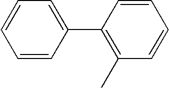 | 382 |
| 47 | 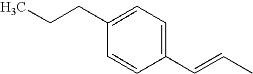 | 374 |
| 48 | 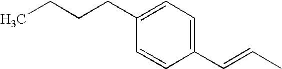 | 388 |
| 49 | 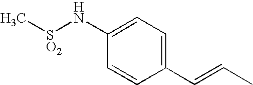 | 425 |
| 50 | 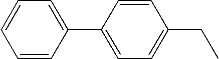 | 396 |
| 51 | 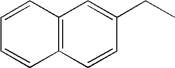 | 370 |
| 52 | 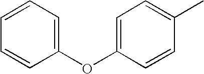 | 398 |
| 53 | 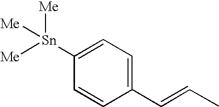 | 496 |
| 54 | 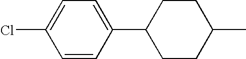 | 422 |
| 55 | 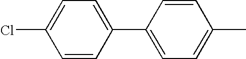 | 416 |
| 56 | 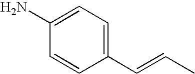 | 347 |
| 57 | 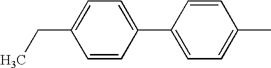 | 410 |
| 58 | 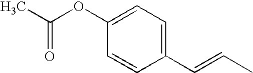 | 390 |
| 59 | 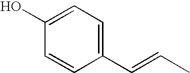 | 348 |
| 60 | 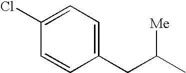 | 382 |
| 61 | 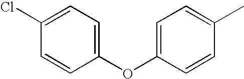 | 432 |
| 62 | 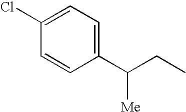 | 382 |
| 63 | 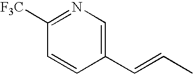 | 401 |
| 64 | 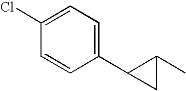 | 380 |
| | isomer A | |
| 65 | 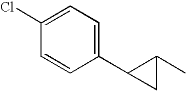 | 380 |
| | isomer B | |
| 66 | 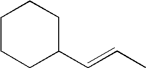 | 338 |
| 67 | 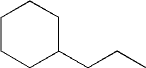 | 340 |
| 68 | 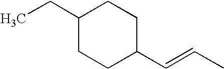 | 366 |
| 69 | 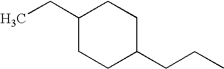 | 368 |
| 70 | 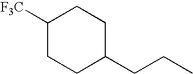 | 408 |
| 71 | 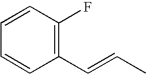 | 350 |
| 72 | 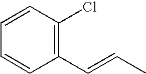 | 366 |
| 73 | 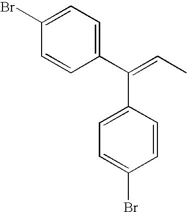 | 566 |
| 74 | 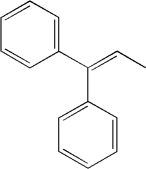 | 408 |
| 75 | 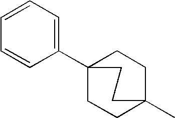 | 414 |
| 76 | 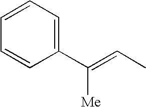 | 346 |
| 77 | 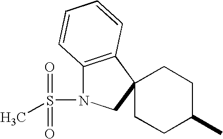 | 493 |
| 78 | 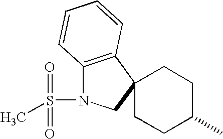 | 493 |

Following procedures similar to those described above for Example 1, the following compounds were prepared from the appropriate starting materials.

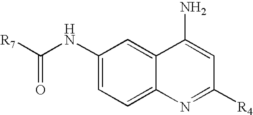

| | | | Parent |
|---|---|---|---|
| | | | Ion |
| | | | (MH+) |
| Ex. # | R₇ | R₄ | m/z |
| 79 | 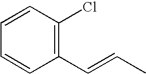 | 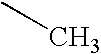 | 339 |
| 80 | 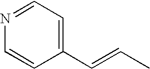 | 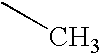 | 305 |
| 81 | 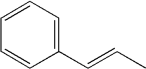 | 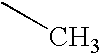 | 304 |
| 82 | 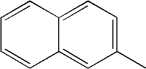 | 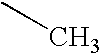 | 328 |
| 83 | 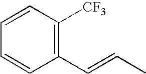 | 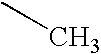 | 372 |
| 84 | 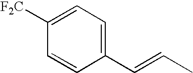 | 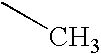 | 372 |
| 85 | 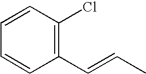 | 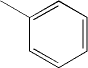 | 400 |
| 86 | 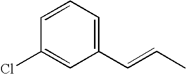 | 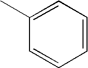 | 400 |
| 87 | 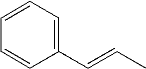 | 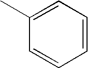 | 366 |
| 88 | 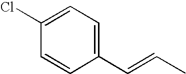 | 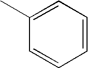 | 400 |
| 89 | 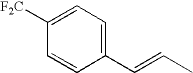 | 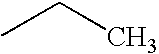 | 386 |
| 90 | 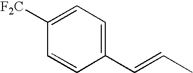 | 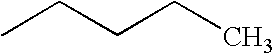 | 414 |
| 91 | 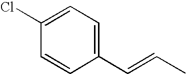 | 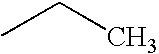 | 352 |
| 92 | 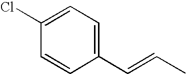 | 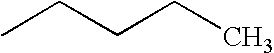 | 380 |
| 93 | 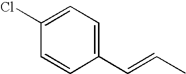 | 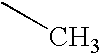 | 338 |
| 94 | 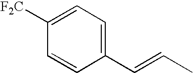 | 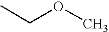 | 402 |
| 95 | 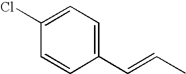 | 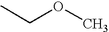 | 368 |
| 96 | 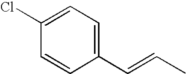 | 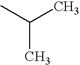 | 366 |
| 97 | 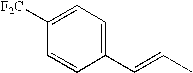 | 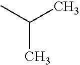 | 400 |
| 98 | 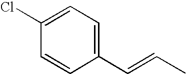 | 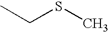 | 384 |
| 99 | 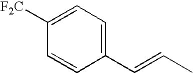 | 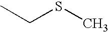 | 418 |
| 100 | 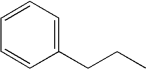 | 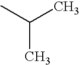 | 334 |
| 101 | 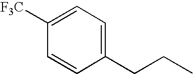 | 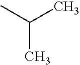 | 402 |
| 102 | 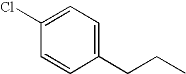 | 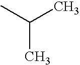 | 368 |
| 103 | 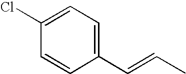 | 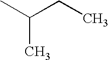 | 380 |
| 104 | 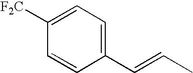 | 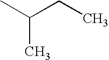 | 414 |
| 105 | 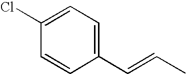 | 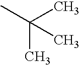 | 380 |
| 106 | 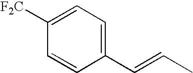 | 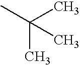 | 414 |
| 107 | 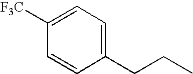 | 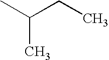 | 416 |
| 108 | 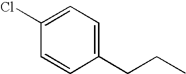 | 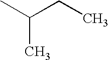 | 382 |
| 109 | 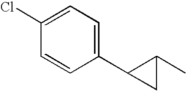 | 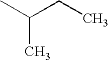 | 394 |
| 110 | 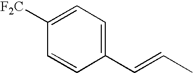 | 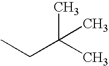 | 428 |
| 111 | 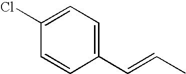 | 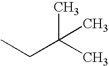 | 394 |
| 112 | 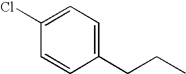 | 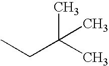 | 396 |
| 113 | 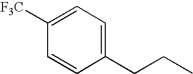 | 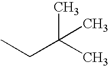 | 430 |
| 114 | 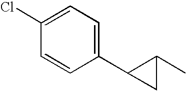 | 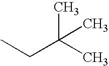 | 408 |
| | (isomer A) | | |
| 115 | 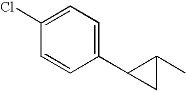 | 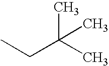 | 408 |
| | (isomer B) | | |
| 116 | 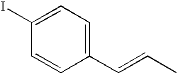 | 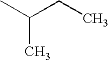 | 472 |
| 117 | 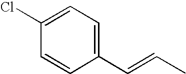 | 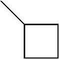 | 378 |
| 118 | 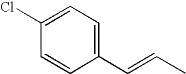 | 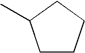 | 392 |
| 119 | 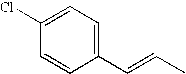 | 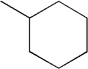 | 406 |
| 120 | 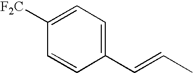 | 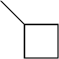 | 412 |
| 121 | 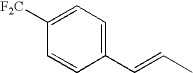 | 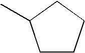 | 426 |
| 122 | 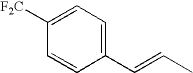 | 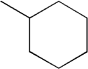 | 440 |
| 123 | 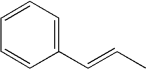 | 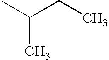 | 346 |

### EXAMPLE 124

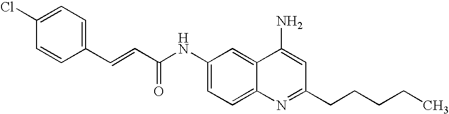

### (2E)-(4-amino-2-pentylquinolin-6-yl)-3-(4-chlorophenyl)prop-2-enamide

### Step A: Preparation of methyl (2E)-3-{[4-(acetylamino)phenyl]amino}oct-2-enoate

A mixture of N-(4-aminophenyl)acetamide (8.9 g, 59 mmol), methyl oct-2-ynoate (10 g, 64.8 mmol), anhydrous potassium fluoride (1 g, 17 mmol) in 100 mL anhydrous N,N-dimethylformamide was purged with nitrogen then heated at 50° overnight. After approximately 18 h, the reaction mixture was cooled to r.t., and filtered. The filtrate was added to 100 mL water, transferred to a separatory funnel and extracted with diethyl ether (5×100 mL). The ether extracts were combined, dried over sodium sulfate, filtered and the solvent removed under vacuum. The resulting dark oil was purified by column chromatography on silica gel eluting with ethyl acetate/hexane gradient (1:2 to 100:0) to afford the product as a brown solid.

### Step B: Preparation of N-(4-hydroxy-2-pentylquinolin-6-yl)acetamide

The product (2.0 g) from Step A was mixed with 20 mL of diphenylether. The mixture was heated with a heating mantle at 260° for 0.25 h then cooled to r.t. The reaction mixture was diluted with EtOAc (25 mL) and the resulting solid was collected by filtration, washed with EtOAc to give a brown solid, MS: m/z 273 (MH⁺), which was used directly in the next step.

### Step C: Preparation of N-(4-methoxy-2-pentylquinolin-6-yl)acetamide

The crude product (0.9 g) from Step B and dimethylsulfate (0.4 mL, 4 mmol) were mixed in toluene (50 mL) and heated at 60° for 4 h. The reaction mixture was cooled to r.t., and the solvent removed under vacuum. The residue was purified by preparative thin layer chromatography eluting with EtOAc/hexanes (1:1) to afford the product as a brown solid, MS: m/z 287 (MH⁺).

### Step D: Preparation of N-(4-amino-2-pentylquinolin-6-yl)acetamide

An intimate mixture of the crude product (0.45 g) from Step C and ammonium acetate (0.6 g, 52 mmol) were heated at 135° for 4 h. The reaction mixture was cooled to r.t. and partitioned between 15 mL 2N aq. NaOH and 15 mL EtOAc. The aqueous layer was extracted with EtOAc (2×10 mL). The organic extracts were combined, dried over sodium sulfate, filtered, and the solvent removed under vacuum. The residue was purified by preparative thin layer chromatography eluting with CH₂Cl₂/MeOH (9:1) to provide the product as a brown semi-solid, MS: m/z 272 (MH⁺).

### Step E: Preparation of 2-pentylquinoline-4,6-diamine

The product (225 mg) from Step D was combined with 3 mL conc. HCl, heated at 90° for 0.5 h, and then cooled to r.t. The mixture was concentrated under vacuum then partitioned between 2N aq. sodium hydroxide (5 mL) and EtOAc. The aqueous mixture was transferred to a separatory funnel and extracted with excess EtOAc. The organic layers were combined, dried with a drying agent and the solvent removed under vacuum. The residue was purified by preparative thin layer chromatography eluting with CH₂Cl₂/MeOH (9:1) to afford the product as a brown semi-solid, MS: m/z 230 (MH⁺).

### Step F: Preparation of (2E)-N-(4-Amino-2-pentylguinolin-6-yl)-3-(4-chlorophenyl)prop-2-enamide

The product was prepared from the product of Step E (25 mg, 0.3 mmol) and (2E)-3-(4-chlorophenyl)prop-2-enoyl chloride Example 1, Step F, 33 mg, 0.16 mmol) according to the procedure for Example 1, Step G. The product was obtained as an amber solid, MS: m/z 394 (MH⁺).

Following procedures similar to those. described above for Example 124, the following compounds were prepared from the appropriate starting materials:

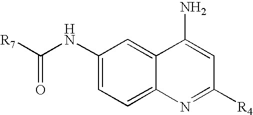

| Ex # | R₇ | R₄ | Parent Ion (MH+) m/z |
|---|---|---|---|
| 125 | 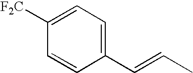 | 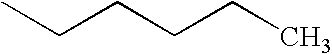 | 428 |
| 126 | 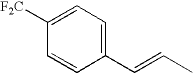 | 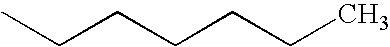 | 442 |
| 127 | 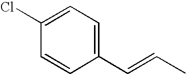 | 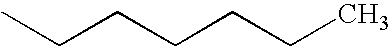 | 408 |

### EXAMPLE 128

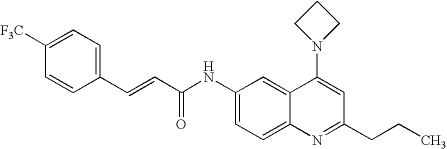

### (2E)-N-(4-azetidin-1-yl-2-propylquinolin-6-yl)-3-[4-(trifluoromethyl)phenyl]prop-2-enamide

### Step A: Preparatiuon of ethyl (2E)-3-[(4-nitrophenyl)amino]hex-2-enoate

A mixture of 4-nitroaniline (15 g, 109 mmol), ethyl 3-oxohexanoate (10 g, 95 mmol) and p-toluenesulfonic acid (0.5 g, 2.6 mmol) toluene was heated at reflux in a flask equipped with a Dean-Stark apparatus and cooling condenser. After the theoretical amount of water was collected, the solvent was removed under vacuum. The residue was used without further purification in the subsequent reaction.

### Step B: Preparation of 6-nitro-2-propylquinolin-4-ol

The crude product from Step A was mixed with diphenylether and the resulting mixture was heated with a heating mantle at 250° for 0.5 h then cooled to r.t. The resulting solid was collected by filtration, washed with EtOAc to give a solid, which was used directly in the next step.

### Step C: Preparation of 4-chloro-6-nitro-2-propylquinoline

The crude product (2.3 g) from Step B and phosphorous oxychloride (10 mL) were heated at 80° for 0.5 h. The reaction mixture was cooled to r.t., poured carefully onto ice with shaking to decompose the excess POCl₃. The mixture was made basic by addition of 5N aq. NaOH. The aqueous layer was extracted with excess EtOAc, the organic layers were combined, dried, filtered and the solvent removed under vacuum to afford the product as a solid, MS: m/z 251 (MH⁺).

### Step D: Preparation of 4-azetidin-1-yl-6-nitro-2-propylquinoline

A mixture of the crude product (0.2 g) from Step C and azetidine (0.25 g, 52 mmol) in methanol was heated at 80° in a sealed tube overnight. The reaction mixture was cooled to r.t. and the solvent removed under vacuum. The residue was purified by column chromatography eluting with EtOAc/hexanes (1:3) to provide the product, MS: m/z 272 (MH⁺).

### Step E: Preparation of 4-azetidin-1-yl-2-propylquinolin-6-amine

The product (170 mg) from Step D was combined with FeCl₃.6H₂O (catalytic amount), carbon (110 mg) in methanol. The mixture was heated at 70° for 0.25 h then hydrazine (0.25 mL) was added. The mixture was heated at reflux for 2.5 h, cooled to r.t., and the solids filtered. The filtrate was concentrated under vacuum, then treated with 6N aq. sodium hydroxide and methanol. The methanol was removed under vacuum. The aqueous mixture was transferred to a separatory funnel and extracted with excess EtOAc. The organic layers were combined, dried with a drying agent, and the solvent removed under vacuum to afford the product, MS: m/z 242 (MH⁺).

### Step F: Preparation of (2E)-3-[(4-trifluoromethyl)phenyl]prop-2-enoyl chloride

the product was prepared from (2E)-3-[(4-trifluoromethyl)phenyl]prop-2-enoic acid according to the procedure for Example 1, Step F.

### Step G: Preparation of (2E)-N-(4-azetidin-1-yl-2-propylquinolin-6-yl)-3-[4-(trifluoromethyl)phenyl]prop-2-enamide

The product was prepared from the product of Step E (15 mg) and (2E)-3-[(4-trifluoromethyl)phenyl]prop-2-enoyl chloride (Step F, 20 mg) according to the procedure for Example 1, Step G. The product was obtained as a solid, MS: m/z 440 (MH⁺).

Following procedures similar to those described above for Example 128, the following compounds were prepared from the appropriate starting materials:

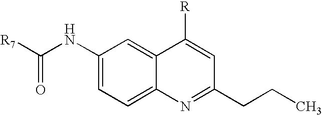

| Ex. # | R₇ | R = —NR₁R₂ | Parent Ion m/z |
|---|---|---|---|
| 129 | 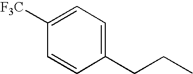 | 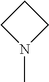 | 442 |
| 130 | 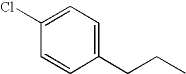 | 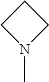 | 408 |
| 131 | 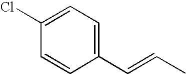 | 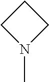 | 406 |
| 132 | 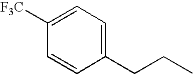 | 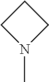 | 442 |
| 133 | 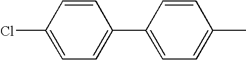 | 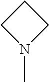 | 456 |
| 134 | 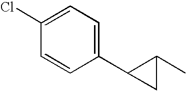 | 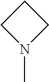 | 420 |
| | isomer A | | |
| 135 | 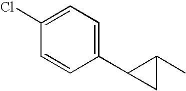 | 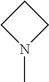 | 420 |
| | isomer A | | |
| 136 | 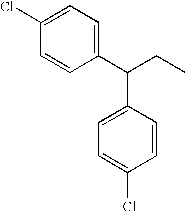 | 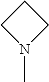 | 518 |
| 137 | 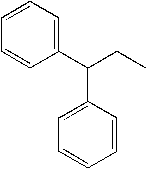 | 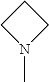 | 450 |
| 138 | 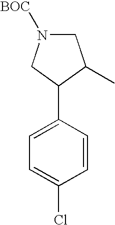 | 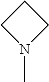 | 549 |
| 139 | 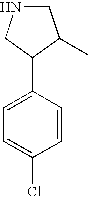 | 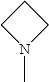 | 449 |
| 140 | 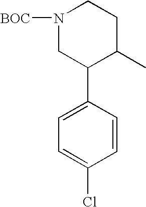 | 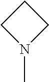 | 563 |
| 141 | 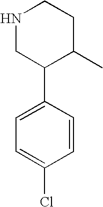 | 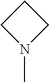 | 463 |
| 142 | 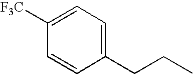 | 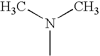 | 430 |
| 143 | 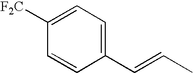 | 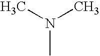 | 428 |
| 144 | 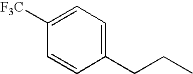 | 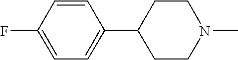 | 564 |
| 145 | 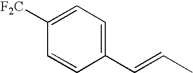 | 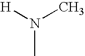 | 414 |
| 146 | 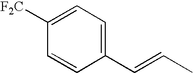 | 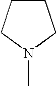 | 454 |
| 147 | 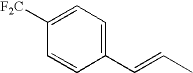 | 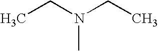 | 456 |
| 148 | 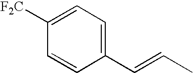 | 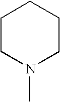 | 468 |
| 149 | 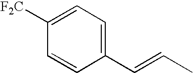 | 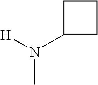 | 454 |
| 150 | 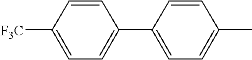 | 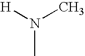 | 464 |
| 151 | 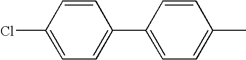 | 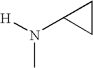 | 456 |
| 152 | 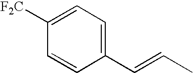 | 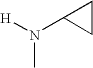 | 440 |
| 153 | 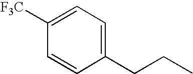 | 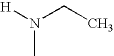 | 408 |
| 154 | 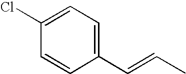 | 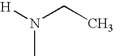 | 394 |
| 155 | 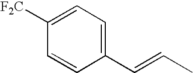 | 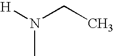 | 406 |

### EXAMPLE 156

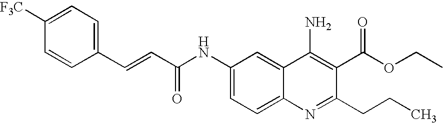

### Ethyl 4-amino-2-propyl-6-({(2E)-3-[4-(trifluoromethyl)phenyl]prop-2-enoyl}amino)quinoline-3-carboxylate

### Step A: Ethyl 4-amino-6-nitro-2-propylquinoline-3-carboxylate

To a stirred solution of ethyl 3-oxohexanoate (3.2 mL, 20 mmol) in toluene under nitrogen atmosphere was added 2-amino-5-nitrobenzonitrile (2.4 g, 14.5 mmol) followed by tin(IV) chloride (4.6 mL, 39 mmol). The resulting mixture was stirred at r.t. for 0.5 h then heated at reflux for 3 h. The reaction mixture was cooled to r.t., and the solvent removed under vacuum. To the residue was added saturated aq. sodium carbonate. The mixture was stirred until decomposition of the tin(IV) chloride was complete. The mixture was transferred to-a separatory funnel and extracted with excess EtOAc. The extracts were combined, dried over a drying agent, filtered and the solvent removed under vacuum. The residue was passed through a pad of silica gel eluting with EtOAc to provide the product as a yellow solid, which was used in the next step.

### Step B: Ethyl 4,6-diamino-2-propylquinoline-3arboxylate

The product was prepared from ethyl 4-amino-6-nitro-2-propylquinoline-3-carboxylate (Step A) according to the procedure for Example 128, Step E, MS: m/z 274 (MH⁺).

### Step C: Ethyl 4-amino-2-propyl-6-({(2E)-3-[4-(trifluoromethyl)phenyl]prop-2-enoyl}amino)quinoline-3-carboxylate

The product was prepared from ethyl 4,6-diamino-2-propylquinoline-3-carboxylate (Step B) and (2E)-3-[(4-trifluoromethyl)phenyl]prop-2-enoyl chloride (Example 128, Step F) according to the procedure for Example 1, Step G, MS: m/z 472 (MH⁺).

Following procedures similar to those described above for Example 156, the following compounds were prepared from the appropriate starting materials or by functional group manipulation of intermediates or products here-in or above.

| | | Parent Ion |
|---|---|---|
| Ex. # | Structure | (MH+) m/z |
| 157 | 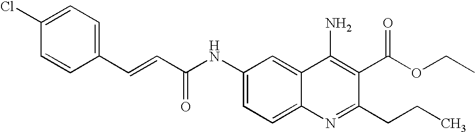 | 412 |
| 158 | 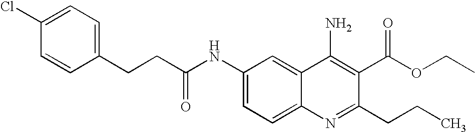 | 440 |
| 159 | 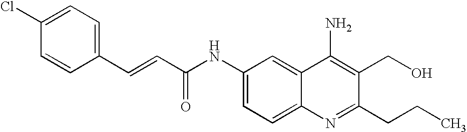 | 396 |
| 160 | 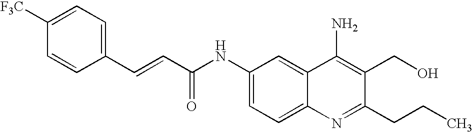 | 430 |
| 161 | 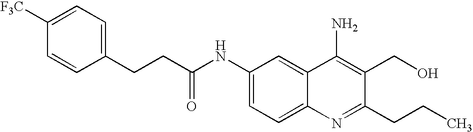 | 432 |
| 162 | 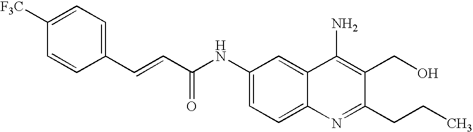 | 444 |
| 163 | 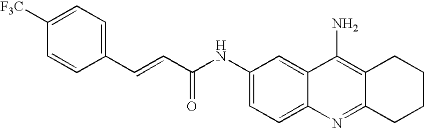 | 412 |
| 164 | 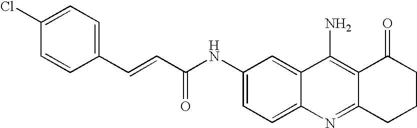 | 392 |
| 165 | 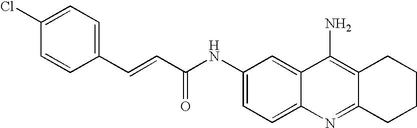 | 378 |
| 166 | 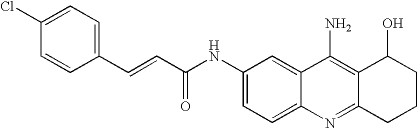 | 394 |

### EXAMPLE 167

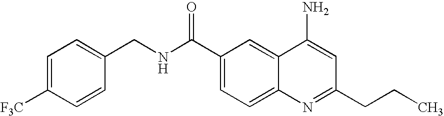

### 4-Amino-N-[4-(trifluoromethyl)benzyl]-2-propylquinoline-6-carboxamide

### Step A: Ethyl 4-{[(1E)-3-ethoxy-3-oxo-1-propylprop-1-enyl]amino}benzoate The product was prepared from ethyl 4-aminobenzoate and ethyl 3-oxohexanoate according to the procedure for Example 1, Step A.

### Step B: Ethyl 4-hydroxy-2-propylquinoline-6-carboxylate

The product was prepared from ethyl 4-{[(1E)-3-ethoxy-3-oxo-1-propylprop-1-enyl]amino}benzoate (Step A) according to the procedure for Example 1, Step B.

### Step C: Ethyl 4-methoxy-2-propylquinoline-6-carboxylate

The product was prepared from ethyl 4-hydroxy-2-propylquinoline-6-carboxylate (Step B) according to the procedure for Example 1, Step C.

### Step D: 4-Methoxy-2-propylquinoline-6-carboxylic acid

A mixture of ethyl 4-methoxy-2-propylquinoline-6-carboxylate (Step C), KOH (15 mg) in 0.5 mL water and 5 mL ethanol was heated at reflux for 3 h. The mixture was cooled to r.t., diluted with water, acidified with aq. HCl and extracted with excess EtOAc. The extracts were combined, dried and solvent removed under vacuum to provide the product which was used in the next Step without further purification.

### Step F: 4-Methoxy-2-propyl-N-[4-(trifluoromethyl)benzyl]quinoline-6-carboxamide

To a solution of 4-methoxy-2-propylquinoline-6-carboxylic acid (Step D, 18 mg, 0.07 mmol) in anhydrous methylene chloride (3 mL) and anhydrous N,N-dimethylformamide (1.5 mL) was added EDC (1.5 eq.), HOBT (1.0 eq.) and 4-(trifluoromethyl)benzylamine (30 mg, 2.3 eq.). The reaction mixture was stirred at r.t. for 3 days. The mixture was quenched with water and extracted with excess EtOAc. The combined extracts were dried over a drying agent filtered and the solvent removed under vacuum. The residue was purified by preparative TLC eluting with EtOAc to afford the the product.

### Step G: 4-Amino-N-[4-(trifluoromethyl)benzyl]-2-propylquinoline-6-carboxamide

The product, MS: m/z 388, was prepared from 4-methoxy-2-propyl-N-[4-(trifluoromethyl)benzyl]quinoline-6-carboxamide (Step F) according to the procedure for Example 1, Step G.

Using procedures analogous to those described above the following Examples were prepared from the appropriate starting materials.

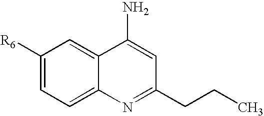

| | | Parent Ion |
|---|---|---|
| Ex. # | R₆ | (MH+) m/z |
| 168 | 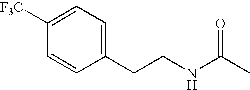 | 402 |
| 169 | 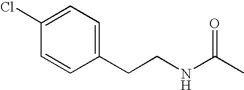 | 368 |
| 170 | 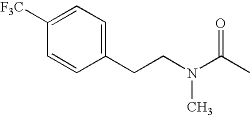 | 416 |
| 171 | 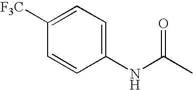 | 374 |
| 172 | 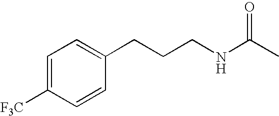 | 416 |
| 173 | 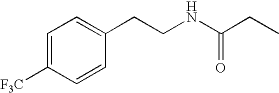 | 416 |
| 174 | 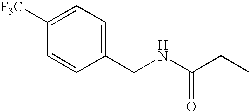 | 402 |
| 175 | 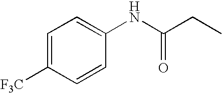 | 388 |

### EXAMPLE 176

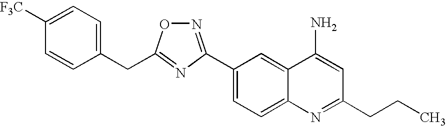

### 2-Propyl-6-{5-[4-(trifluoromethyl)benzyl]-1,2,4-oxadiazol-3-yl}quinolin-4-amine

### Step A: Ethyl (2E)-3-[(4-cyanophenyl)amino]hex-2-enoate

The product was prepared from 4-aminobenzonitrile and ethyl 3-oxohexanoate according to the procedure for Example 1, Step A.

### Step B: 4-Hydroxy-2-propylquinoline-6-carbonitrile

The product was prepared from ethyl (2E)-3-[(4-cyanophenyl)amino]hex-2-enoate (Step A) according to the procedure for Example 1, Step B.

### Step C: 4-Methoxy-2-propylquinoline-6-carbonitrile

The product MS: m/z 227, was prepared from 4-hydroxy-2-propylquinoline-6-carbonitrile (Step B) according to the procedure for Example 1, Step C.

### Step D: N′-hydroxy-4-methoxy-2-propylquinoline-6-carboximidamide Or N-hydroxy-4-methoxy-2-propylquinoline-6-carboximidamide

A mixture of 4-methoxy-2-propylquinoline-6-carbonitrile (Step C, 900 mg), hydroxylamine hydrochloride (3 eq.), sodium carbonate (3 eq.) in 3 mL water and 10 mL ethanol was stirred overnight. The mixture was diluted with water, extracted with excess EtOAc. The extracts were combined, dried and solvent removed under vacuum. The residue was triturated with EtOAc and the solvent decanted away to provide the product (610 mg)which was used in the next step without further purification.

### Step E: 4-Methoxy-6-{5-[4-(trifluoromethyl)benzyl]-1,2,4-oxadiazol-3-yl}-2-propylquinoline

To a mixture of the product of Step D, (130 mg) in anhydrous diglyme (10 mL) was added 4-trifluoromethylphenylacetic acid (2 eq.), EDC (2 eq.) and HOBT (1.0 eq.). The reaction mixture was stirred at r.t. overnight. After approximately 18 hr, the mixture was heated at 130 for 2 hr. The mixture was cooled to r.t., quenched with water and extracted with excess EtOAc. The combined extracts were dried over a drying agent filtered and the solvent removed under vacuum. The residue was purified by preparative TLC eluting with EtOAc to afford the product (115 mg).

### Step F: 2-Propyl-6-{5-[4-(trifluoromethyl)benzyl]-1,2,4-oxadiazol-3-yl}quinolin-4-amine

The product (58 mg), MS: m/z 413, was prepared from 4-methoxy-6-{5-[4-(trifluoromethyl)benzyl]-1,2,4-oxadiazol-3-yl}-2-propylquinoline (70 mg, Step E) according to the procedure for Example 1, Step D,

Using procedures analogous to those described above the following Examples were prepared from the appropriate starting materials:

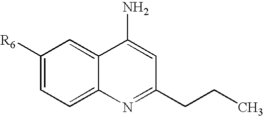

| | | Parent Ion |
|---|---|---|
| Ex. # | R₆ | (MH+) m/z |
| 177 | 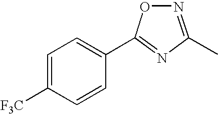 | 399 |
| 178 | 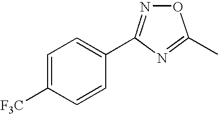 | 399 |
| 179 | 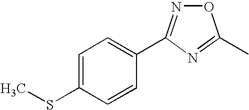 | 377 |
| 180 | 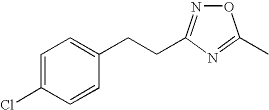 | 393 |
| 181 | 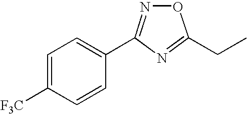 | 413 |
| 182 | 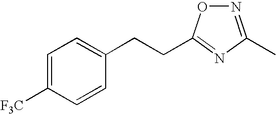 | 427 |
| 183 | 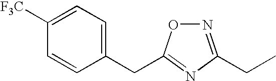 | 427 |
| 184 | 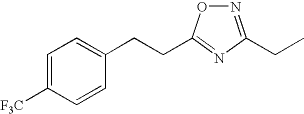 | 441 |
| 185 | 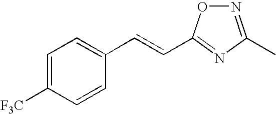 | 425 |
| 186 | 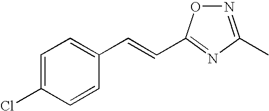 | 391 |
| 187 | 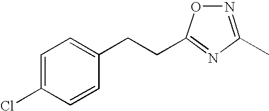 | 393 |
| 188 | 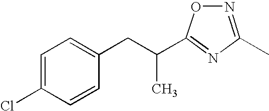 | 407 |
| 189 | 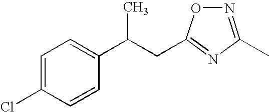 | 407 |
| 190 | 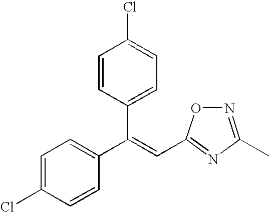 | 501 |
| 191 | 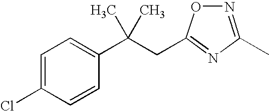 | 421 |

### EXAMPLE 192

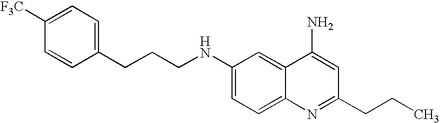

### 2-Propyl-N⁶-{3-[4-(trifluoromethyl)phenyl]propyl}quinoline-4,6-diamine

### Step A: 2-Propyl-N⁶-{3-[4-(trifluoromethyl)phenyl]propyl}quinoline-4,6-diamine

To a solution of N-(4-amino-2-propylquinolin-6-yl)-3-[4-(trifluoromethyl)phenyl]propanamide (86 mg, 0.2 mmol, Example 26) in 6 mL THF under nitrogen atmosphere was added lithium aluminium hydride (400 mg, 10.5 mmol). The reaction mixture was heated at reflux for 3 h, then cooled in an ice bath. The reaction was quenched by careful addition of water (1 mL) followed by 5N aq. potassium hydroxide (1 mL). The viscous mixture was triturated with excess EtOAc and the solvents decanted away. This was repeated three times. The organic layers were combined, dried over magnesium sulfate, filtered and the solvent removed under vacuum. The residue was purified by preparative TLC eluting with CHCl₃/2N NH3 in MeOH (9:1) to afford the product as a tan solid, MS: m/z 388 (MH⁺).

Using chemistry known to those skilled in the art, the following compounds were made using analogous procedures used to prepare Example 192 shown above or by functional group manipulation of intermediates and/or examples shown above.

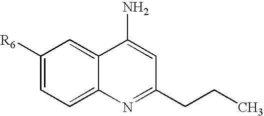

| | | Parent Ion |
|---|---|---|
| Ex. # | R₆ | (MH+) m/z |
| 193 | 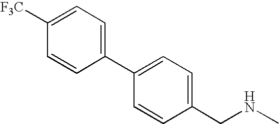 | 436 |
| 194A | 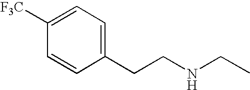 | 388 |
| 194B | 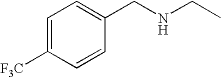 | 374 |
| 195 | 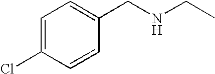 | 340 |
| 196 | 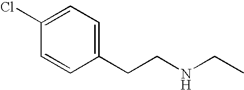 | 354 |
| 197 | 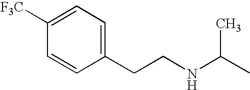 | 402 |
| 198 | 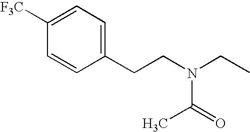 | 430 |
| 199 | 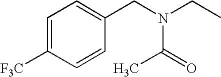 | 416 |
| 200 | 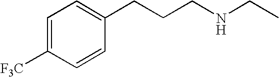 | 402 |

Using chemistry known to those skilled in the art, the following compounds were made using analogous procedures used to prepare the examples shown above or by functional group manipulation of intermediates and/or examples shown above.

| Ex. # | Structure | Parent Ion (MH+) m/z |
|---|---|---|
| 201 | 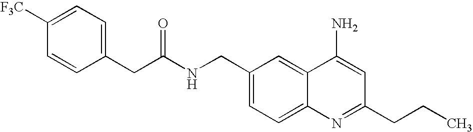 | 402 |
| 202 | 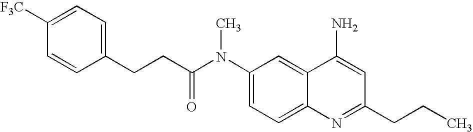 | 416 |
| 203 | 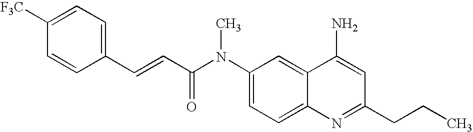 | 414 |
| 204 | 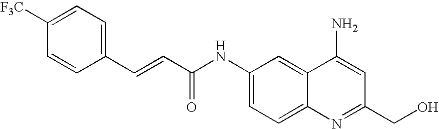 | 388 |
| 205 | 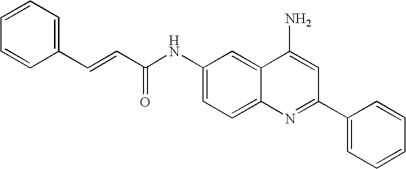 | 366 |
| 206 | 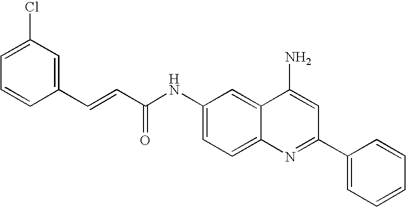 | 400 |
| 207 | 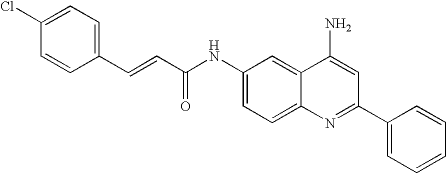 | 400 |
| 208 | 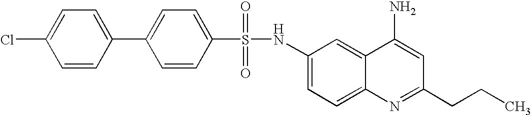 | 452 |

### EXAMPLE 209

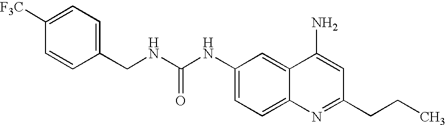

### N-(4-amino-2-propylquinolin-6-yl)-N′-[4-(trifluoromethyl)benzyl]urea

To a solution of triphosgene (27 mg, 0.09 mmol) in methylene chloride (0.6 mL) under nitrogen atmosphere was added a mixture of 4-trifluoromethylbenzylamine (0.04 mL, 0.28 mmol) and N,N-diisopropylethylamine (0.11 mL) over 15 minutes by syringe pump. The resulting mixture was stired at r.t. for 0.25 h and the solvent removed under vacuum to provide a solid. The solid was added a solution of 2-propylquinoline-4,6-diamine (52 mg, 0.26 mmol; Example 1 Step E) in acetic acid (1.5 mL). The reaction mixture was stirred at r.t. for 3.5 h and the solvent removed under vacuum. The residue was purified by preparative TLC eluting with CHCl₃/2N NH₃ in MeOH (9:1) to afford the product as a solid, MS: m/z 403 (MH⁺).

Using chemistry known to those skilled in the art, the following compounds were made using analogous procedures used to prepare the examples shown above or by functional group manipulation of intermediates and/or examples shown above.

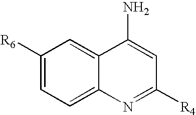

| Ex. # | R₆ | R₄ | Parent Ion (MH+) m/z |
|---|---|---|---|
| 210 | 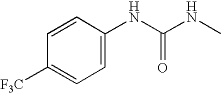 | 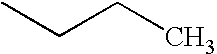 | 389 |
| 211 | 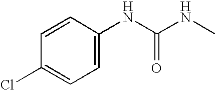 | 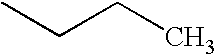 | 355 |
| 212 | 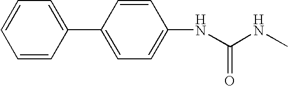 | 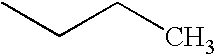 | 397 |
| 213 | 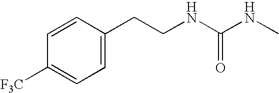 | 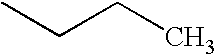 | 417 |
| 214 | 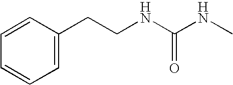 | 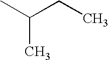 | 349 |
| 215 | 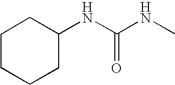 | 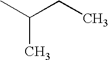 | 327 |
| 216 | 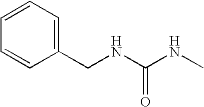 | 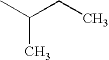 | 335 |
| 217 | 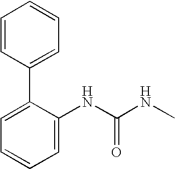 | 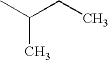 | 397 |
| 218 | 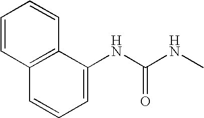 | 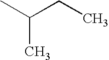 | 371 |
| 219 | 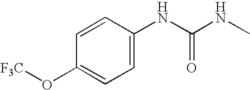 | 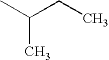 | 405 |
| 220 | 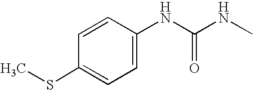 | 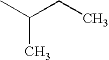 | 367 |
| 221 | 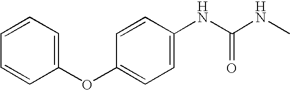 | 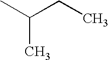 | 413 |
| 222 | 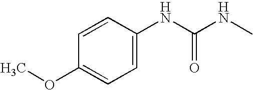 | 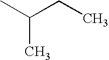 | 351 |
| 223 | 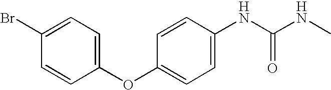 | 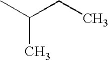 | 493 |
| 224 | 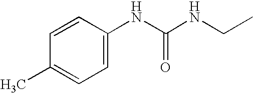 | 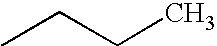 | 349 |
| 225 | 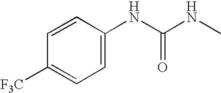 | 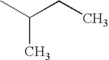 | 389 |
| 226 | 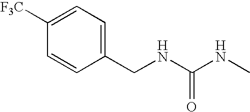 | 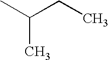 | 403 |
| 227 | 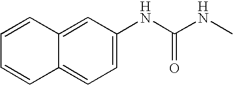 | 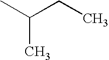 | 371 |
| 228 | 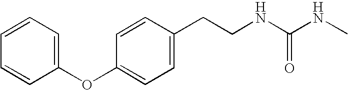 | 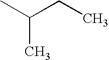 | 441 |
| 229 | 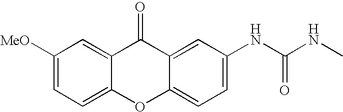 | 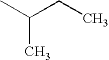 | 469 |
| 230 | 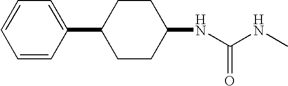 | 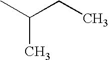 | 403 |
| 231 | 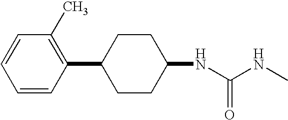 | 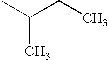 | 417 |
| 232 | 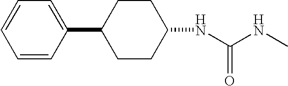 | 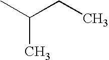 | 403 |
| 233 | 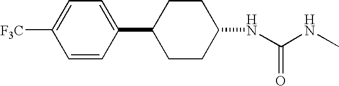 | 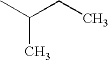 | 471 |
| 234 | 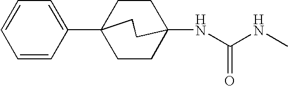 | 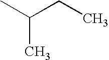 | 429 |
| 235 | 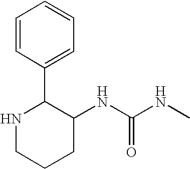 | 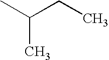 | 404 |
| 236 | 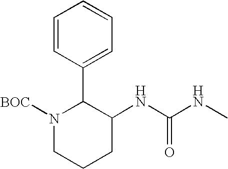 | 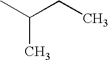 | 504 |
| 237 | 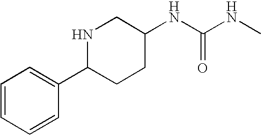 | 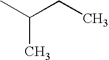 | 404 |
| 238 | 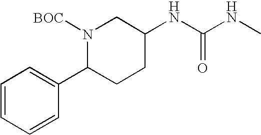 | 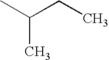 | 504 |
| 239 | 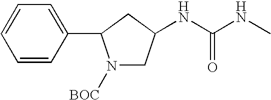 | 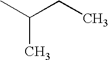 | 490 |
| 240 | 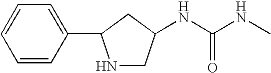 | 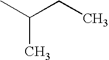 | 390 |
| 241 | 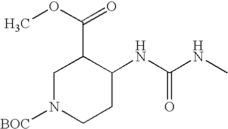 | 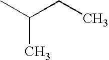 | 486 |
| 242 | 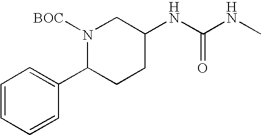 | 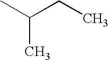 | 504 |
| 243 | 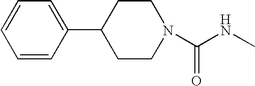 | 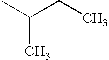 | 389 |
| 244 | 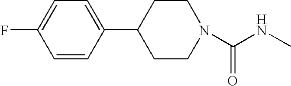 | 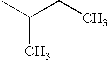 | 407 |

### BIOLOGICAL ASSAYS

MCH-1R and MCH-2R Radioligand Binding Assays

Membrane binding assays were performed on transiently-transfected COS-7 cells expressing human MCH-2R from the plasmid vector pCI-neo (Promega, Madison, Wis., on a Chinese hamster ovary (CHO) cell line stably expressing the MCH-2R from the plasmid vector pEFI/V5-HisB (Invitrogen, Carlsbad, Calif.), or a CHO cell line stably expressing human MCH-1R from pcDNA3.1. For transient expression, COS-7 cells were cultured in Dulbecco's modified Eagle medium (Gibco BRL, Rockville, Md.) with 10% heat inactivated fetal calf serum. A suspension of 7×10⁶ COS-7 cells were transfected with 20 μg of pCI-neo/MCH-2R plasmid by electroporation (26) and cells were harvested after 60-72 hours. Membranes were prepared from transient and stable transfectants by hypotonic lysis, frozen in liquid nitrogen, and stored at −80° C. A scintillation proximity assay (SPA) was developed to measure the specific binding of [¹²⁵I]-[Phe¹³Tyr¹⁹]-hMCH. SPA were carried out using wheat-germ agglutinin-polyvinyltoluene beads (Amersham Corp., Arlington Heights, Ill.), in 96-well OptiPlates (Packard, Meriden, Conn.). Each well contained 0.25 mg of SPA beads, 1-10 μg of membrane protein, and 200 μL binding buffer (50 mM Tris pH 7.4, 10 mM MgCl_{2, 2} mM EDTA, 12% glycerol, 0.1% BSA). Binding buffer contained 50 mM Tris pH 7.4, 8 mM MgCl₂, 12% glycerol, 0.1% BSA (Sigma, St. Louis, Mo.) and protease inhibitors: 4 μg/mL of leupeptin (Sigma, St. Louis, Mo.), 40 μg/mL of Bacitracin (Sigma, St. Louis, Mo.), 5 μg/mL of Aprotinin (Roche Molecular Biochem., Indianapolis, Ind.), 0.05M AEBSF (Roche Molecular Biochem., Indianapolis, Ind.), and 5 mM Phosphoramidon (Boeringer Mannheim). Assays were optimized with respect to membrane preparations: for CHO/MCH-1R membranes, 1 μg of membranes per well yielded a>6× specific binding window and for COS or CHO MCH-2R membranes, 8 μg of membrane protein yielded a window of about 3×. Specific binding is defined as the difference between total binding and non-specific binding conducted in the presence of 500 nM unlabeled hMCH. Beads were coated with membranes for 20 minutes and dispensed to the 96 wells, various concentrations of test compounds in DMSO were added (final DMSO concentration 1%-2%), then 25 nCi of [¹²⁵I]-[Phe¹³Tyr¹⁹]-hMCH (˜2000 Ci/mmol; NEN Life Sciences, Boston, Mass.) was added to the wells. After equilibrating at r.t. for 3 hours, the plates were read in a TopCount (Packard, Meriden, Conn.). IC₅₀ calculations were performed using Prism 3.0 (GraphPad Software, San Diego, Calif.). The IC₅₀ values were measured in three different experiments. A filter-based assay was also used for MCH-2R in 96-well plates. Total volume per binding assay point was 200 μL. Binding conditions were 50 mM Tris pH 7.4, 10 mM MgCl_{2, 2} mM EDTA 200 μg/mL bacitracin, 1 μM phosphoramidon, 2.5 to 5 μg protein, with and without 10 μM MCH unlabeled peptide as a competitor. Dose response curves were from 10 μM in 5 fold or 3-fold dilution series for 11 points. The mixture was shaken for 5 minutes on a platform shaker, and incubated at r.t. for 1 hour. Filter plates were presoaked in 1% PEI. The binding reaction was harvested onto filters using Packard Filtermate harvester (Meriden, Conn.). The filters were then washed in 50 mM Tris pH 7.4, 10 mM MgCl₂, 2 mM EDTA, 0.04% Tween 20, 6-8 times per plate. The plates were dried for 20 minutes at 55° C. or overnight at r.t. 30 μL microscintillant was added per well and counted for 1.5-3 minutes in inverted format on Packard TopCount. IC₅₀ calculations were performed using Prism 3.0 (GraphPad Software, San Diego, Calif.).

Functional Assay for MCH-1R and MCH-2R

The aequorin bioluminescence assay is a reliable test for identifying G-protein-coupled receptors which couple through the G protein subunit family consisting of G_{q} and Gᵢᵢ which leads to the activation of phospholipase C, mobilization of intracellular calcium, and activation of protein kinase C. Stable cell lines expressing either the MCH-1R or the MCH-2R and the aequorin reporter protein were used. The assay was performed using a Luminoskan RT luminometer (Labsystems Inc., Gaithersburg, Md.) controlled by custom software written for a Macintosh PowerPC 6100. 293AEQ17/MCH-1R(or MCH-2R) cells were cultured for 72 h and the apo-aequorin in the cells was charged for 1 h with coelenterazine (10 μM) under reducing conditions (300 M reduced glutathione) in ECB buffer (140 mM NaCl, 20 mM KCl, 20 mM HEPES-NaOH, pH 7.4, 5 mM glucose, 1 mM MgCl₂, 1 mM CaCl₂, 0.1 mg/mL bovine serum albumin). The cells were harvested, washed once in ECB medium, and resuspended to 500 000 cells/mL. 100 μL of cell suspension (corresponding to 5×10⁴ cells) was then injected into the test plate containing the test ligands, and the integrated light emission was recorded over 30 s, in 0.5-s units. 20 μL of lysis buffer (0.1% final Triton X-100 concentration) was then injected and the integrated light emission recorded over 10 s, in 0.5-s units. To detect antagonists, test ligands were pre-incubated for ˜10 minutes at varying concentrations prior to injection on the test ligand plate containing MCH agonists. The “fractional response” values for each well were calculated by taking the ratio of the integrated response to the initial challenge to the total integrated luminescence including the Triton X-100 lysis response. The functional EC₅₀ values were measured in three separate assays.

Selective MCH-1R antagonist compounds of the present invention have IC₅₀ affinities for the MCH-1R receptor between 0.1 and 10000 nM, are at least 20× selective for the MCH-1R receptor over the MCH-2R receptor, and are functional antagonists lacking agonist activity at the MCH-1R receptor.

References:

MCH-1R (Human):

Lakaye et al., “Cloning of the rat brain cDNA encoding for the SLC-1 G protein-coupled receptor reveals the presence of an intron in the gene,” Biochim. Biophys Acta; 1401(2):216-20 (1998).

Saito et al., “Molecular characterization of the melanin-concentrating-hormone receptor”, Nature;.400(6741):265-9 (1999).

Chambers et al., “Melanin-concentrating hormone is the cognate ligand for the orphan G-protein-coupled receptor SLC-1”, Nature; 400(6741):261-5 (1999). MCH-2R (human):

Sailer et al., “Identification and characterization of a second melanin-concentrating hormone receptor, MCH-2R”, Proc. Natl. Acad. Sci. U S A; 98(13):7564-9 (2001).

In vivo Food Intake Models.

1) Overnight food intake. Sprague Dawley rats are injected intracerebroventricularly with a test compound in 400 nL of 50% propylene glycol/artificial cerebrospinal fluid one hour prior to onset of dark cycle (12 hours). Food intake is determined using a computerized system in which each rat's food is placed on a computer monitored balance. Cumulative food intake for 16 hours post compound administration is measured.

2) Food intake in diet induced obese mice. Male C57/B16J mice maintained on a high fat diet (60% fat calories) for 6.5 months from 4 weeks of age are dosed intraperitoneally with test compound. Food intake and body weight are measured over an eight day period. Biochemical parameters relating to obesity, including leptin, insulin, triglyceride, free fatty acid, cholesterol and serum glucose levels are determined.

While the invention has been described and illustrated in reference to certain preferred embodiments thereof, those skilled in the art will appreciate that various changes, modifications and substitutions can be made therein without departing from the spirit and scope of the invention. For example, effective dosages other than the preferred doses as set forth hereinabove may be applicable as a consequence of variations in the responsiveness of the mammal being treated for obesity, diabetes, or for other indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be limited only by the scope of the claims that follow and that such claims be interpreted as broadly as is reasonable.

1. A compound of structural formula (I): 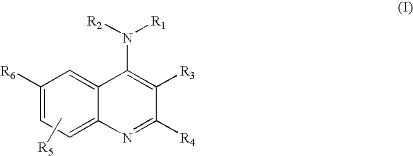
wherein:
R¹ and R² are independently selected from the group consisting of:
(1) hydrogen,
(2) C₁₋₆ alkyl,
(3) C₂₋₆ alkenyl,
(4) C₂₋₆ alkynyl,
(5) cycloalkyl-C₀₋₆ alkyl,
(6) heterocycloalkyl-C₀₋₁₀ alkyl,
(7) aryl-C₀₋₁₀ alkyl, and
(8) heteroaryl-C₀₋₁₀ alkyl;
wherein alkyl, alkenyl, and alkynyl, moieties above are optionally substituted with one to four substituents independently selected from R^{a}; and wherein cycloalkyl, heterocycloalkyl aryl and heteroaryl moieties above are optionally substituted with one to four substituents independently selected from R^{b}; and wherein sulfur-containing heterocyclic rings may be mono- or di-oxidized on the sulfur atom;
or, R¹ and R² together with the nitrogen atom to which they are attached, form a 4- to 10-membered bridged or unbridged heterocyclic ring, optionally containing one or two additional heteroatoms selected from N, S, and O, optionally having one or more degrees of unsaturation, optionally fused to a 6-membered heteroaromatic or aromatic ring, either unsubstituted or substituted with one to four substituents independently selected from R^{b}; and wherein sulfur-containing heterocyclic rings may be mono- or di-oxidized on the sulfur atom;
R³ and R⁴ are independently selected from the group consisting of:
(1) hydrogen,
(2) halogen,
(3) C₁₋₈ alkyl,
(4) perfluoro C₁₋₆ alkyl,
(5) C₂₋₆ alkenyl,
(6) C₂₋₆ alkynyl,
(7) cycloalkyl,
(8) cycloalkyl-C₁₋₆ alkyl,
(9) cycloheteroalkyl,
(10) cycloheteroalkyl-C₁₋₆ alkyl,
(11) aryl,
(12) aryl-C₁₋₆ alkyl,
(13) heteroaryl,
(14) heteroaryl-C₁₋₆ alkyl,
(15) —OR⁷,
(16) —NR⁷R⁷,
(17) —CO₂R⁷,
(18) cyano,and
(19) —C(O)NR⁷R⁷;
wherein alkyl, alkenyl and alkynyl, moieties above are optionally substituted with one to four substituents independently selected from R^{a}; and wherein cycloalkyl, heterocycloalkyl, aryl and heteroaryl moieties above are optionally substituted with one to four substituents independently selected from R^{b}; and wherein sulfur-containing heterocyclic rings may be mono- or di-oxidized on the sulfur atom;
or, R³ and R⁴ together with the ring carbon atoms to which they are attached, form a 5- to 7-membered heterocycloalkyl or cycloalkyl ring, either unsubstituted or substituted with one to four substituents independently selected from R^{b};
R⁵ is selected from:
(1) hydrogen,
(2) halogen,
(3) C₁₋₆ alkyl,
(4) perfluoro C₁₋₆ alkyl,
(5) —OR⁷, and
(6) —NR⁷R⁷;
R⁶ is selected from the group consisting of:
(1) —(CH₂)ₙ—R⁷,
(2) —(CH₂)ₙ-aryl-R⁷,
(3) —(CH₂)ₙ-heteroaryl-R⁷,
(4) —(CH₂)ₙ-heterocycloalkyl-R⁷,
(5) —(CH₂)ₙC≡N,
(6) —(CH₂)ₙCON(R⁷)₂,
(7) —(CH₂)ₙCO₂R⁷,
(8) —(CH₂)ₙCOR⁷,
(9) —(CH₂)ₙNR⁷C(O)R⁷,
(10) —(CH₂)ₙNR⁷C(O)(CH₂)ₙSR⁷
(11) —(CH₂)ₙNR⁷CO₂R⁷,
(12) —(CH₂)ₙNR⁷C(O)N(R⁷)₂,
(13) —(CH₂)ₙNR⁷SO₂R⁷,
(14) —(CH₂)ₙS(O)ₚR⁷,
(15) —(CH₂)ₙSO₂N(R⁷)₂,
(16) —(CH₂)ₙOR⁷,
(17) —(CH₂)ₙOC(O)R⁷,
(18) —(CH₂)ₙOC(O)OR⁷,
(19) —(CH₂)ₙOC(O)N(R⁷)₂,
(20) —(CH₂)ₙN(R⁷)₂, and
(21) —(CH₂)ₙNR⁷SO₂N(R⁷)₂,
wherein one or two of the hydrogen atoms in (CH₂)ₙ may be substituted with R^{a};
R⁷ is independently selected at each occurrence from the group consisting of:
(1) hydrogen,
(2) C₁₋₆ alkyl,
(3) aryl,
(4) heteroaryl,
(5) cycloalkyl,
(6) heterocycloalkyl,
(7) aryl C₁₋₃ alkyl,
(8) heteroaryl C₁₋₃ alkyl,
(9) cycloalkyl C₁₋₃ alkyl,
(10) heterocycloalkyl C₁₋₃ alkyl,
(11) aryl C₂₋₃ alkenyl,
(12) heteroaryl C₂₋₃ alkenyl,
(13) cycloalkyl C₂₋₃ alkenyl, and
(14) heterocycloalkyl C₂₋₃ alkenyl,
wherein the alkyl and alkenyl moieties are optionally substituted with one to four substituents selected from R^{a}; and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom;
each R^{a} is independently selected from:
(1) —OR^{d},
(2) —NR^{d}S(O)ₘR^{d},
(3) —NO₂,
(4) halogen,
(5) —S(O)ₘR^{d},
(6) —SR^{d},
(7) —S(O)₂OR^{d},
(8) —S(O)ₚN(R^{d})₂,
(9) —N(R^{d})₂,
(10) —O(CR^{d}R^{d})ₙN(R^{d})₂,
(11) —C(O)R^{d},
(12) —CO₂R^{d},
(13) —CO₂(CR^{d}R^{d})ₙCON(R^{d})₂,
(14) —OC(O)R^{d},
(15) —CN,
(16) —C(O)N(R^{d})₂,
(17) —NR^{d}C(O)R^{d},
(18) —OC(O)N(R^{d})₂,
(19) —NR^{d}C(O)OR^{d},
(20) —NR^{d}C(O)N(R^{d})₂,
(21) —CR^{d}(N—OR^{d}),
(22) —CF₃,
(23) cycloalkyl,
(24) cycloheteroalkyl, and
(25) oxo;
each R^{b} is independently selected from:
(1) R^{a},
(2) —Sn(CH₃)₃,
(3) C₁₋₁₀ alkyl,
(4) C₂₋₁₀ alkenyl,
(5) C₂₋₁₀ alkynyl,
(6) heteroaryl,
(7) aryl, and
(8) aryl-C₁₋₁₀ alkyl;
wherein alkyl, alkenyl, alkynyl, cycloalkyl, cycloheteroalkyl, heteroaryl, and aryl are optionally substituted with one to four substituents selected from a group independently selected from R^{c};
each R^{c} is independently selected from:
(1) halogen,
(2) amino,
(3) carboxy,
(4) C₁₋₄ alkyl,
(5) C₁₋₄ alkoxy,
(6) aryl,
(7) aryl C₁₋₄ alkyl,
(8) hydroxy,
(9) —CF₃,
(10) —OC(O)C₁₋₄ alkyl,
(11) —OC(O)N(R^{d})₂, and
(12) aryloxy;
R^{d} is independently selected from hydrogen, C₁₋₆ alkyl, C₂₋₆ alkenyl; C₂₋₆ alkynyl; cycloalkyl; cycloalkyl-C₁₋₆ alkyl; cycloheteroalkyl; cycloheteroalkyl-C₁₋₆ alkyl; aryl; heteroaryl; aryl-C₁₋₆ alkyl; and heteroaryl-C₁₋₆ alkyl;
wherein the alkyl, alkenyl, alkynyl, cycloalkyl, cycloheteroalkyl, heteroaryl, and aryl in R^{d} are optionally substituted with one to four substituents independently selected from R^{e};
each R^{e} is selected from halo, methyl, methoxy, trifluoromethyl, trifluoromethoxy, and hydroxy;
m is selected from 1 and 2;
n is selected from: 0, 1, 2, 3, 4, and 5;
p is selected from 0, 1, and 2;
and pharmaceutically acceptable salts thereof.

2. The compound according to claim 1, wherein:
R¹ and R² are independently selected from the group consisting of:
(1) hydrogen,
(2) C₁₋₆ alkyl,
(3) C₂₋₆ alkenyl,
(4) cycloalkyl-C₀₋₆ alkyl,
(5) heterocycloalkyl-C₀₋₆ alkyl,
(6) aryl-C₀₋₆ alkyl, and
(7) heteroaryl-C₀₋₁₀ alkyl;
wherein alkyl and alkenyl moieties above are optionally substituted with one to three substituents independently selected from R^{a}; and wherein cycloalkyl, heterocycloalkyl, aryl and heteroaryl moieties above are optionally substituted with one to three substituents independently selected from R^{b};
or, R¹ and R² together with the nitrogen atom to which they are attached, form a 4- to 10-membered bridged or unbridged heterocyclic ring, optionally containing one additional heteroatom selected from N, S, and O, optionally having one or more degrees of unsaturation, optionally fused to a 6-membered heteroaromatic or aromatic ring, either unsubstituted or substituted with an R^{b} substituent;
R³ and R⁴ are independently selected from the group consisting of:
(1) hydrogen,
(2) halogen,
(3) C₁₋₈ alkyl,
(4) trifluoromethyl,
(5) C₂₋₆ alkenyl,
(6) cycloalkyl,
(7) cycloalkyl-C₁₋₆ alkyl,
(8) cycloheteroalkyl,
(9) cycloheteroalkyl-C₁₋₆ alkyl,
(10) aryl,
(11) aryl-C₁₋₆ alkyl,
(12) heteroaryl,
(13) heteroaryl-C₁₋₆ alkyl,
(14) —OR⁷,
(15) —NR⁷R⁷,
(16) —CO₂R⁷, and
(17) —C(O)NR⁷R⁷;
wherein alkyl and alkenyl moieties above are optionally substituted with one to three substituents independently selected from R^{a}; and wherein cycloalkyl, heterocycloalkyl, aryl and heteroaryl moieties above are optionally substituted with an R^{b} substituent;
or, R³ and R⁴ together with the ring carbon atoms to which they are attached, form a 5- to 7-membered heterocycloalkyl or cycloalkyl ring, either unsubstituted or substituted with an R^{b} substituent;
R⁵ is selected from:
(1) hydrogen,
(2) halogen,
(3) methyl,
(4) trifluoromethyl,
(5) hydroxy,
(6) methoxy,
(7) phenoxy,
(8) —NH₂,
(9) —NH(CH₃), and
(10) —N(CH₃)₂;
R⁶ is selected from the group consisting of:
(1) —(CH₂)ₙ—R⁷,
(2) —(CH₂)ₙ-aryl-R⁷,
(3) —(CH₂)ₙ-heteroaryl-R⁷,
(4) —(CH₂)ₙ-heterocycloalkyl-R⁷,
(5) —(CH₂)ₙC≡N,
(6) —(CH₂)ₙCON(R⁷)₂,
(7) —(CH₂)ₙCO₂R⁷,
(8) —(CH₂)ₙCOR⁷,
(9) —(CH₂)ₙNR⁷C(O)R⁷,
(10) —(CH₂)ₙNR⁷C(O)(CH₂)ₙSR⁷
(11) —(CH₂)ₙNR⁷CO₂R⁷,
(12) —(CH₂)ₙNR⁷C(O)N(R⁷)₂,
(13) —(CH₂)ₙNR⁷SO₂R⁷,
(14) —(CH₂)ₙS(O)ₚR⁷,
(15) —(CH₂)ₙSO₂N(R⁷)₂,
(16) —(CH₂)ₙOR⁷,
(17) —(CH₂)ₙOC(O)R⁷,
(18) —(CH₂)ₙOC(O)OR⁷,
(19) —(CH₂)ₙOC(O)N(R⁷)₂,
(20) —(CH₂)ₙN(R⁷)₂, and
(21) —(CH₂)ₙNR⁷SO₂N(R⁷)₂,
wherein one or two of the hydrogen atoms in (CH₂)ₙ may be substituted with R^{a};
R⁷ is independently selected at each occurrence from the group consisting of
(1) hydrogen,
(2) C₁₋₆ alkyl,
(3) aryl,
(4) heteroaryl,
(5) cycloalkyl,
(6) heterocycloalkyl,
(7) aryl C₁₋₃ alkyl,
(8) heteroaryl C₁₋₃ alkyl,
(9) cycloalkyl C₁₋₃ alkyl,
(10) heterocycloalkyl C₁₋₃ alkyl,
(11) aryl C₂₋₃ alkenyl,
(12) heteroaryl C₂₋₃ alkenyl,
(13) cycloalkyl C₂₋₃ alkenyl, and
(14) heterocycloalkyl-C₂₋₃ alkenyl,
wherein the alkyl and alkenyl moieties are optionally substituted with one to four substituents selected from R^{a}; and wherein the aryl, heteroaryl, cycloalkyl and heterocycloalkyl moieties are independently substituted with one to four substituents selected from R^{b}; and wherein sulfur-containing heterocyclic rings may be mono- or di-oxidized on the sulfur atom;
each R^{a} is independently selected from:
(1) —OR^{d},
(2) —NR^{d}S(O)ₘR^{d},
(3) —NO₂,
(4) halogen,
(5) —S(O)ₘR^{d},
(6) —SR^{d},
(7) —S(O)₂OR^{d},
(8) —S(O)ₚN(R^{d})₂,
(9) —N(R^{d})₂,
(10) —O(CR^{d}R^{d})ₙN(R^{d})₂,
(11) —C(O)R^{d},
(12) —CO₂R^{d},
(13) —CO₂(CR^{d}R^{d})ₙCON(R^{d})₂,
(14) —OC(O)R^{d},
(15) —CN,
(16) —C(O)N(R^{d})₂,
(17) —NR^{d}C(O)R^{d},
(18) —OC(O)N(R^{d})₂,
(19) —NR^{d}C(O)OR^{d},
(20) —NR^{d}C(O)N(R^{d})₂,
(21) —CR^{d}(N—OR^{d}),
(22) —CF₃,
(23) cycloalkyl,
(24) cycloheteroalkyl, and
(25) oxo;
each R^{b} is independently selected from:
(1) R^{a},
(2) —Sn(CH₃)₃,
(3) C₁₋₁₀ alkyl,
(4) C₂₋₁₀ alkenyl,
(5) heteroaryl,
(6) aryl, and
(7) aryl-C₁₋₁₀ alkyl;
wherein alkyl, alkenyl, cycloalkyl, cycloheteroalkyl, heteroaryl, and aryl are optionally substituted with one to four substituents selected from a group independently selected from R^{c};
each R^{c} is independently selected from:
(1) halogen,
(2) amino,
(3) carboxy,
(4) C₁₋₄ alkyl,
(5) C₁₋₄ alkoxy,
(6) aryl,
(7) aryl C₁₋₄ alkyl-,
(8) hydroxy,
(9) —CF₃,
(10) —OC(O)C₁₋₄ alkyl,
( 11) —OC(O)N(R^{d})₂, and
(12) aryloxy;
R^{d}is independently selected from hydrogen, C₁₋₆ alkyl, C₂₋₆ alkenyl; C₂₋₆ alkynyl; cycloalkyl; cycloalkyl-C₁₋₆ alkyl; cycloheteroalkyl; cycloheteroalkyl-C₁₋₆alkyl; aryl; heteroaryl; aryl-C₁₋₆ alkyl; and heteroaryl-C₁₋₆ alkyl;
wherein the alkyl, alkenyl, alkynyl, cycloalkyl, cycloheteroalkyl, heteroaryl, and aryl in R^{d} are optionally substituted with one to two substituents independently selected from a R^{e};
each R^{e} is selected from halo, methyl, methoxy, trifluoromethyl, trifluoromethoxy, and hydroxy;
m is selected from 1 and 2;
n is selected from: 0, 1, 2, 3, 4, and 5;
p is selected from 0, 1, and 2;
and pharmaceutically acceptable salts thereof.

3. The compound according to claim 2, wherein:
R¹ is selected from the group consisting of:
(1) hydrogen, and
(2) C₁₋₆ alkyl, optionally substituted with one to three substituents independently selected from R^{a};
R² is selected from the group consisting of:
(1) hydrogen,
(2) C₁₋₆ alkyl,
(3) cycloalkyl-C₀₋₆ alkyl,
(4) heterocycloalkyl-C₀₋₆ alkyl,
(5) aryl-C₀₋₁₀ alkyl, and
(6) heteroaryl-C₀₋₁₀ alkyl;
wherein alkyl moieties above are optionally substituted with one to three substituents independently selected from R^{a}; and wherein cycloalkyl, heterocycloalkyl, aryl and heteroaryl moieties above are optionally substituted with one to three substituents independently selected from R^{b};
or, R¹ and R² together with the nitrogen atom to which they are attached, form a 4- to 10-membered bridged or unbridged heterocyclic ring, optionally containing one additional heteroatom selected from N, S, and O, either unsubstituted or substituted with an R^{b} substituent;
R³ is selected from the group consisting of:
(1) hydrogen,
(2) halogen,
(3) C₁₋₈ alkyl,
(4) trifluoromethyl,
(5) —OH,
(6) —OCH₃,
(7) —NH₂,
(8) —CO₂R⁷, and
(9) —C(O)NH₂;
wherein alkyl moieties above are optionally substituted with one to two substituents independently selected from R^{a};
R⁴ is selected from the group consisting of:
(1) hydrogen,
(2) halogen,
(3) C₁₋₈ alkyl,
(4) trifluoromethyl,
(5) cycloalkyl,
(6) cycloheteroalkyl,
(7) aryl,
(8) aryl-C₁₋₆ alkyl,
(9) heteroaryl,
(10) —OH,
(11) —OCH,
(12) —NH₂,
(13) —CO₂R⁷, and
(14) —C(O)NH₂;
wherein alkyl moieties above are optionally substituted with one to four substituents independently selected from R^{a}; and wherein cycloalkyl, heterocycloalkyl, aryl and heteroaryl moieties above are optionally substituted with an R^{b} substituent;
or, R³ and R⁴ together with the ring carbon atoms to which they are attached, form a 5- to 7-membered cycloalkyl ring, either unsubstituted or substituted with an R^{b} substituent;
R⁵ is selected from:
(1) hydrogen,
(2) halogen,
(3) methyl,
(4) trifluoromethyl,
(5) hydroxy,
(6) methoxy,
(7) phenoxy,
(8) —NH₂,
(9) —NH(CH₃), and
(10) —N(CH₃)₂;
R⁶ is selected from the group consisting of:
(1) —(CH₂)ₙ—R⁷,
(2) —(CH₂)ₙ-aryl-R⁷,
(3) —(CH₂)ₙ-heteroaryl-R⁷,
(4) —(CH₂)ₙ-heterocycloalkyl-R⁷,
(5) —(CH₂)ₙC≡N,
(6) —(CH₂)ₙCON(R⁷)₂,
(7) —(CH₂)ₙCO₂R⁷,
(8) —(CH₂)ₙCOR⁷,
(9) —(CH₂)ₙNR⁷C(O)R⁷,
(10) —(CH₂)ₙNR⁷C(O)(CH₂)ₙSR⁷
(11) —(CH₂)ₙNR⁷CO₂R⁷,
(12) —(CH₂)ₙNR⁷C(O)N(R⁷)₂,
(13) —(CH₂)ₙNR⁷SO₂R⁷,
(14) —(CH₂)ₙS(O)ₚR⁷,
(15) —(CH₂)ₙSO₂N(R⁷)₂,
(16) —(CH₂)ₙOR⁷,
(17) —(CH₂)ₙOC(O)R⁷,
(18) —(CH₂)ₙOC(O)OR⁷,
(19) —(CH₂)ₙOC(O)N(R⁷)₂,
(20) —(CH₂)ₙN(R⁷)₂, and
(21) —(CH₂)ₙNR⁷SO₂N(R⁷)₂,
wherein one or two of the hydrogen atoms in (CH₂)n may be substituted with R^{a};
R⁷ is independently selected at each occurrence from the group consisting of
(1) hydrogen,
(2) C₁₋₆ alkyl,
(3) aryl,
(4) heteroaryl,
(5) cycloalkyl,
(6) heterocycloalkyl,
(7) aryl C₁₋₃ alkyl,
(8) heteroaryl C₁₋₃ alkyl,
(9) cycloalkyl C₁₋₃ alkyl,
(10) heterocycloalkyl C₁₋₃ alkyl,
(11) aryl C₂₋₃ alkenyl,
(12) heteroaryl C₂₋₃ alkenyl,
(13) cycloalkyl C₂₋₃ alkenyl, and
(14) heterocycloalkyl C₂₋₃ alkenyl,
wherein the alkyl and alkenyl moieties are optionally substituted with one to three substituents selected from R^{a}; and wherein the aryl, heteroaryl, cycloalkyl and heterocycloalkyl moieties are independently substituted with one to three substituents selected from R^{b}; and wherein sulfur-containing heterocyclic rings may be mono- or di-oxidized on the sulfur atom;
each R^{a} is independently selected from:
(1) —OR^{d},
(2) —NR^{d}S(O)ₘR^{d},
(3) —NO₂,
(4) halogen,
(5) —S(O)ₘR^{d},
(6) —SR^{d},
(7) —S(O)₂OR^{d},
(8) —S(O)ₚN(R^{d})₂,
(9) —N(R^{d})₂,
(10) —O(CR^{d}R^{d})ₙN(R^{d})₂,
(11) —C(O)R^{d},
(12) —CO₂R^{d},
(13) —CO₂(CR^{d}R^{d})ₙCON(R^{d})₂,
(14) —OC(O)R^{d},
(15) —CN,
(16) —C(O)N(R^{d})₂,
(17) —NR^{d}C(O)R^{d},
(18) —OC(O)N(R^{d})₂,
(19) —NR^{d}C(O)OR^{d},
(20) —NR^{d}C(O)N(R^{d})₂,
(21) —CR^{d}(N—OR^{d}),
(22) —CF₃,
(23) cycloalkyl,
(24) cycloheteroalkyl, and
(25) oxo;
each R^{b} is independently selected from:
(1) R^{a},
(2) —Sn(CH₃)₃,
(3) C₁₋₁₀ alkyl,
(4) C₂₋₁₀ alkenyl,
(5) heteroaryl,
(6) aryl, and
(7) aryl-C₁₋₁₀ alkyl;
wherein alkyl, alkenyl, cycloalkyl, cycloheteroalkyl, heteroaryl, and aryl moieties in R^{a} and R^{b} are optionally substituted with one to four substituents selected from a group independently selected from R^{c};
each R^{c} is independently selected from:
(1) halogen,
(2) amino,
(3) carboxy,
(4) C₁₋₄ alkyl,
(5) C₁₋₄ alkoxy,
(6) aryl,
(7) aryl C₁₋₄ alkyl-,
(8) hydroxy,
(9) —CF₃,
(10) —OC(O)C₁₋₄ alkyl,
(11) —OC(O)N(R^{d})₂, and
(12) aryloxy;
R^{d}is independently selected from hydrogen, C₁₋₆ alkyl, C₂₋₆ alkenyl; C₂₋₆ alkynyl; cycloalkyl; cycloalkyl-C₁₋₆ alkyl; cycloheteroalkyl; cycloheteroalkyl-C₁₋₆ alkyl; aryl; heteroaryl; aryl-C₁₋₆ alkyl; and heteroaryl-C₁₋₆ alkyl;
wherein the alkyl, alkenyl, alkynyl, cycloalkyl, cycloheteroalkyl, heteroaryl, and aryl in R^{d} are optionally substituted with one to two substituents independently selected from a R^{e};
each R^{e} is selected from halo, methyl, methoxy, trifluoromethyl, trifluoromethoxy, and hydroxy;
m is selected from 1 and 2;
n is selected from: 0, 1, 2, 3, and 4;
p is selected from 0, 1, and 2;
and pharmaceutically acceptable salts thereof.

4. The compound according to claim 3, wherein:
R¹ is selected from the group consisting of:
(1) hydrogen,
(2) methyl,
(3) ethyl, and
(4) propyl,
optionally substituted with one to three substituents independently selected from R^{a};
R² is selected from the group consisting of:
(1) hydrogen,
(2) C₁₋₆ alkyl,
(3) cycloalkyl-C₀₋₆ alkyl,
(4) heterocycloalkyl-C₀₋₆ alkyl,
(5) aryl-C₀₋₆ alkyl, and
wherein alkyl moieties above are optionally substituted with one to three substituents independently selected from R^{a}; and wherein cycloalkyl, heterocycloalkyl, aryl and heteroaryl moieties above are optionally substituted with one to three substituents independently selected from R^{b};
or, R¹ and R² together with the nitrogen atom to which they are attached, form a 4- to 10-membered bridged or unbridged heterocyclic ring, optionally containing one additional heteroatom selected from N, S, and O, either unsubstituted or substituted with an R^{b}substituent;
R³ is selected from the group consisting of:
(1) hydrogen,
(2) halogen,
(3) C₁₋₈ alkyl,
(4) trifluoromethyl,
(5) —OH,
(6) —OCH₃,
(7) —NH₂,
(8) —CO₂H,
(9) —CO₂CH₃,
(10) —CO₂CH₂CH_{3, and}
(11) —C(O)NH₂;
wherein alkyl moieties above are optionally substituted with one to three substituents independently selected from R^{a};
R⁴ is selected from the group consisting of:
(1) C₁₋₈ alkyl,
(2) trifluoromethyl,
(3) cycloalkyl,
(4) cycloheteroalkyl,
(5) aryl,
(6) heteroaryl,
(7) —NH₂,
(8) —CO₂H,
(9) CO₂CH₃, and
(10) —CO₂CH₂CH₃;
wherein alkyl moieties above are optionally substituted with one to three substituents independently selected from R^{a}; and wherein cycloalkyl, heterocycloalkyl, aryl and heteroaryl moieties above are optionally substituted with an R^{b} substituent;
or, R³ and R⁴ together with the ring carbon atoms to which they are attached, form a 5- to 7-membered cycloalkyl ring, either unsubstituted or substituted with oxo or hydroxy;
R⁵ is selected from:
(1) hydrogen,
(2) halogen,
(3) methyl,
(4) trifluoromethyl,
(5) hydroxy, and
(6) methoxy;
R⁶ is selected from the group consisting of:
(1) —(CH₂)ₙ—R⁷,
(2) —(CH₂)ₙ-aryl-R⁷,
(3) —(CH₂)ₙ-heteroaryl-R⁷,
(4) —(CH₂)ₙ-heterocycloalkyl-R⁷,
(5) —(CH₂)ₙCON(R⁷)₂,
(6) —(CH₂)ₙNR⁷C(O)R⁷,
(7) —(CH₂)ₙNR⁷C(O)(CH₂)ₙSR⁷
(8) —(CH₂)ₙNR⁷C(O)N(R⁷)₂,
(9) —(CH₂)ₙNHSO₂R⁷,
(10) —(CH₂)ₙN(R⁷)₂, and
(11) —(CH₂)ₙNR⁷SO₂N(R⁷)₂,
wherein one or two of the hydrogen atoms in (CH₂)ₙ may be substituted with R^{a};
R⁷ is independently selected at each occurrence from the group consisting of
(1) hydrogen,
(2) C₁₋₆ alkyl,
(3) aryl,
(4) heteroaryl,
(5) cycloalkyl,
(6) heterocycloalkyl,
(7) aryl C₁₋₃ alkyl,
(8) heteroaryl C₁₋₃ alkyl,
(9) cycloalkyl C₁₋₃ alkyl,
(10) heterocycloalkyl C₁₋₃ alkyl,
(11) aryl C₂₋₃ alkenyl,
(12) heteroaryl C₂₋₃ alkenyl,
(13) cycloalkyl C₂₋₃ alkenyl, and
(14) heterocycloalkyl C₂₋₃ alkenyl,
wherein the alkyl and alkenyl moieties are optionally substituted with one to three substituents selected from R^{a}; and wherein the aryl, heteroaryl, cycloalkyl and heterocycloalkyl moieties are independently substituted with one to three substituents selected from R^{b}; and wherein sulfur-containing heterocyclic rings may be mono- or di-oxidized on the sulfur atom;
each R^{a} is independently selected from:
(1) —OR^{d},
(2) —NHSO₂CH₃,
(3) —NO₂,
(4) halogen,
(5) —S(O)ₘCH₃,
(6) —SR^{d},
(7) —S(O)₂OR^{d},
(8) —S(O)_{N}(R^{d})₂,
(9) —N(R^{d})₂,
(10) —O(CR^{d}R^{d})ₙN(R^{d})₂,
(11) —C(O)R^{d},
(12) —CO₂R^{d},
(13) —CO₂(CR^{d}R^{d})ₙCON(R^{d})₂,
(14) —OC(O)R^{d},
(15) —CN,
(16) —C(O)N(R^{d})₂,
(17) —NR^{d}C(O)R^{d},
(18) —OC(O)N(R^{d})₂,
(19) —NR^{d}C(O)OR^{d},
(20) —NR^{d}C(O)N(R^{d})₂,
(21) —CR^{d}(N—OR^{d}),
(22) —CF₃,
(23) cycloalkyl,
(24) cycloheteroalkyl, and
(25) oxo;
each R^{b} is independently selected from:
(1) R^{a},
(2) —Sn(CH₃)₃,
(3) C₁₋₆ alkyl,
(4) C₂₋₆ alkenyl,
(5) heteroaryl,
(6) aryl, and
(7) aryl-C₁₋₁₀ alkyl;
wherein alkyl, alkenyl, cycloalkyl, cycloheteroalkyl, heteroaryl, and aryl moieties in R^{a} and R^{b} are optionally substituted with one to four substituents selected from a group independently selected from R^{c};
each R^{c} is independently selected from:
(1) halogen,
(2) amino,
(3) carboxy,
(4) C₁₋₄ alkyl,
(5) C₁₋₄ alkoxy,
(6) aryl,
(7) aryl C₁₋₄ alkyl-,
(8) hydroxy,
(9) —CF₃,
(10) —OC(O)C₁₋₄ alkyl,
(11) —OC(O)N(R^{d})₂, and
(12) aryloxy;
R^{d} is independently selected from hydrogen, C₁₋₆ alkyl, C₂₋₆ alkenyl; C₂₋₆ alkynyl; cycloalkyl; cycloalkyl-C₁₋₆ alkyl; cycloheteroalkyl; cycloheteroalkyl-C₁₋₆ alkyl; aryl; heteroaryl; aryl-C₁₋₆ alkyl; and heteroaryl-C₁₋₆ alkyl;
wherein the alkyl, alkenyl, alkynyl, cycloalkyl, cycloheteroalkyl, heteroaryl, and aryl in R^{d} are optionally substituted with one to two substituents independently selected from a R^{e};
each R^{e} is selected from halogen, methyl, methoxy, trifluoromethyl, trifluoromethoxy, and hydroxy;
m is selected from 1 and 2;
n is selected from: 0, 1, 2, 3, and 4;
p is selected from 0, 1, and 2;
and pharmaceutically acceptable salts thereof.

5. The compound according to claim 4, wherein:
R¹ is selected from the group consisting of:
(1) hydrogen,
(2) methyl,
(3) ethyl, and
(4) propyl,
optionally substituted with one to three substituents independently selected from R^{a};
R² is selected from the group consisting of:
(1) hydrogen,
(2) methyl,
(3) ethyl,
(4) n-propyl,
(5) isopropyl,
(6) t-butyl,
(7) n-butyl,
(8) cyclopropyl,
(9) cyclobutyl,
(10) cyclopentyl,
(11) cyclohexyl,
(12) heterocycloalkyl-C₀₋₆ alkyl, wherein the heterocycloalkyl moiety is selected from azetidinyl, pyrrolidinyl, and pyridyl, and
(13) phenyl-C₀₋₃ alkyl,
wherein alkyl moieties above are optionally substituted with one to three substituents independently selected from R^{a}; and wherein cycloalkyl, heterocycloalkyl, aryl and heteroaryl moieties above are optionally substituted with one to three substituents independently selected from R^{b};
or, R¹ and R² together with the nitrogen atom to which they are attached, form a 4- to 10-membered bridged or unbridged heterocyclic ring, selected from: azetidinyl, pyrrolidinyl, piperidinyl, morpholinyl, 1-thia4-azacyclohexyl, azacycloheptyl, 2-oxa-5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, either unsubstitute or substituted with an R^{b} substituent;
R³ is selected from the group consisting of:
(1) hydrogen,
(2) halogen,
(3) C₁₋₈ alkyl,
(4) trifluoromethyl,
(5) —OH,
(6) —OCH₃,
(7) —NH₂,
(8) —CO₂H,
(9) —CO₂CH₃, and
(10) —CO₂CH₂CH₃;
wherein alkyl moieties above are optionally substituted with one to three substituents independently selected from R^{a};
R⁴ is independently selected from the group consisting of
(1) C₁₋₈ alkyl,
(2) trifluoromethyl,
(3) cyclobutyl,
(4) cyclopentyl,
(5) cyclohexyl,,
(6) phenyl,
(7) —CO₂H,
(8) —CO₂CH₃, and
(9) —CO₂CH₂CH₃;
wherein alkyl moieties above are optionally substituted with one to three substituents independently selected from R^{a}; and wherein cycloalkyl, heterocycloalkyl, aryl and heteroaryl moieties above are optionally substituted with an R^{b} substituent;
or, R³ and R⁴ together with the ring carbon atoms to which they are attached, form a cyclohexyl ring, either unsubstituted or substituted with oxo or hydroxy;
R⁵ is hydrogen;
R⁶ is selected from the group consisting of:
(1) —R⁷,
(2) -heteroaryl-R⁷,
(3) —CONHR⁷,
(4) —CON(R⁷)(CH₃),
(5) —CH₂CONHR⁷,
(6) —CH₂CON(R⁷)(CH₃),
(7) —CH₂NHC(O)R⁷,
(8) —NHC(O)R⁷,
(9) —(CH₂)ₙNHC(O)(CH₂)ₙSR⁷
(10) —(CH₂)ₙNHC(O)N(CH₃)(R⁷),
(11) —(CH₂)ₙNHC(O)NH(R⁷),
(12) —(CH₂)ₙNHSO₂R⁷,
(13) —NH(R⁷),
(14) —N(COCH₃)(R⁷),
(15) —(CH₂)ₙNH(R⁷), and
(16) —(CH₂)ₙN(COCH₃)(R⁷),
wherein one or two of the hydrogen atoms in (CH₂)n may be substituted with R^{a};
R⁷ is independently selected at each occurrence from the group consisting of
(1) hydrogen,
(2) C₁₋₆ alkyl,
(3) aryl, selected from: phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, and 1,4-benzodioxanyl,
(4) heteroaryl, selected from: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, and thienopyridinyl,
(5) cycloalkyl, selected from: cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl,
(6) heterocycloalkyl, selected from: azetidinyl, pyridyl, pyrrolidinyl, piperidinyl, piperazinyl, imidazolidinyl, morpholinyl, 1-thia-4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl,indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl,
(7) aryl C₁₋₃ alkyl, wherein the aryl moiety is selected from: phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, and 1,4-benzodioxanyl,
(8) heteroaryl C₁₋₃ alkyl, wherein the heteroaryl moiety is selected: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, and thienopyridinyl,
(9) cycloalkyl C₁₋₃ alkyl, wherein the cycloalkyl moiety is selected from: cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl,
(10) heterocycloalkyl C₁₋₃ alkyl, wherein the heterocycloalkyl moiety is selected from: azetidinyl, pyridyl, pyrrolidinyl, piperidinyl, piperazinyl, imidazolidinyl, morpholinyl, 1-thia4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl,indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl,
(11) aryl C₂₋₃ alkenyl, wherein the aryl moiety is selected from: phenyl, naphthyl, indanyl, indenyl, indolyl, quinazolinyl, quinolinyl, benzthiazolyl, benzoxazolyl, dihydroindanyl, benzisodiazolyl, spirocyclohexylindolinyl, spiro-(dihydrobenzothiophenyl)piperidinyl, spiro-indolinylpiperidinyl, indolinyl, tetrahydroisoquinolinyl, isoindolinyl, benzothiadiazolyl, benzotriazolyl, 1,3-dihydro-2-benzofuranyl, benzothiophenyl, benzodioxolyl, tetrahydronaphthyl, 2,3-dihydrobenzofuranyl, dihydrobenzopyranyl, and 1,4-benzodioxanyl,
(12) heteroaryl C₂₋₃ alkenyl, wherein the heteroaryl moiety is selected from: pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo[2,3-b]pyridyl, quinolyl, indolyl, isoquinolyl, quinazolinyl, benzisodiazolyl, triazolopyrimidinyl, 5,6,7,8-tetrahydroquinolinyl, 2,1,3-benzothiadiazolyl, and thienopyridinyl,
(13) cycloalkyl C₂₋₃ alkenyl, wherein the cycloalkyl moiety is selected from: cyclopropyl, cyclobutyl, cyclopentyl, cyclohexyl, cycloheptyl, tetrahydronaphthyl, decahydronaphthyl, indanyl, bicyclo[2.2.2]octanyl, tetrahydronaphthyl, and dihydroindanyl, and
(14) heterocycloalkyl C₂₋₃ alkenyl, wherein the heterocycloalkyl moiety is selected from: azetidinyl, pyridyl, pyrrolidinyl, piperidinyl, piperazinyl, imidazolidinyl, morpholinyl, 1-thia4-aza-cyclohexane, 2,5-diazabicyclo[2.2.2]octanyl, 2,3-dihydrofuro[2,3-b]pyridyl, benzoxazinyl, tetrahydroquinolinyl, tetrahydroisoquinolinyl, dihydroindolyl,indolyl, indolinyl, isoindolinyl, 1,3-dihydro-2-benzofuranyl, benzodioxolyl, hexahydrothienopyridinyl, thienopyridinyl, azacycloheptyl, 4,4-spiro[2,3-dihydrobenzothiophen-3,3-yl]piperidinyl, and 4,4-spiro[indoli-3,3-yl]piperidinyl;
wherein the alkyl moieties are optionally substituted with one to three substituents selected from R^{a}; and wherein the aryl, heteroaryl, cycloalkyl and heterocycloalkyl moieties are independently substituted with one to three substituents selected from R^{b}; and wherein sulfur-containing heterocyclic rings may be mono- or di-oxidized on the sulfur atom;
each R^{a} is independently selected from:
(1) —OR^{d},
(2) —NHSO₂CH₃,
(3) —NO₂,
(4) halogen,
(5) —S(O)ₘCH₃,
(6) —SCH₃,
(7) —SCF₃,
(8) —S(O)₂OH,
(9) —S(O)ₚN(R^{d})₂,
(10) —N(CH₃)₂,
(11) —NH₂,
(12) —O(CR^{d}R^{d})ₙN(R^{d})₂,
(13) —C(O)R^{d},
(14) —CO₂H,
(15) —CO₂CH₃,
(16) t-butyloxycarbonyl,
(17) —CO₂(CR^{d}R^{d})ₙCON(R^{d})₂,
(18) —OC(O)R^{d},
(19) —CN,
(20) —C(O)N(R^{d})₂,
(21) —NR^{d}C(O)R^{d},
(22) —OC(O)N(R^{d})₂,
(23) —NR^{d}C(O)OR^{d},
(24) —NR^{d}C(O)N(R^{d})₂,
(25) —CR^{d}(N—OR^{d}),
(26) —CF_{3,}
(27) cycloalkyl,
(28) cycloheteroalkyl, and
(29) oxo;
each R^{b} is independently selected from:
(1) —R^{a},
(2) —Sn(CH₃)₃,
(3) C₁₋₆ alkyl,
(4) C₂₋₆ alkenyl,
(5) heteroaryl,
(6) phenyl, and
(7) phenyl-C₁₋₁₀ alkyl;
wherein alkyl, alkenyl, cycloalkyl, cycloheteroalkyl, heteroaryl, and aryl moieties in R^{a} and R^{b} are optionally substituted with one to four substituents selected from a group independently selected from R^{c};
each R^{c} is independently selected from:
(1) halogen,
(2) amino,
(3) carboxy,
(4) C₁₋₄ alkyl,
(5) C₁₋₄ alkoxy,
(6) aryl,
(7) aryl C₁₋₄ alkyl,
(8) hydroxy,
(9) —CF₃,
(10) —OC(O)C₁₋₄ alkyl,
(11) —OC(O)N(R^{d})₂, and
(12) aryloxy;
R^{d} independently selected from hydrogen, C₁₋₆ alkyl, C₂₋₆ alkenyl; C₂₋₆ alkynyl; cycloalkyl; cycloalkyl-C₁₋₆ alkyl; cycloheteroalkyl; cycloheteroalkyl-C₁₋₆alkyl; aryl; heteroaryl; aryl-C₁₋₆ alkyl; and heteroaryl-C₁₋₆ alkyl;
wherein the alkyl, alkenyl, alkynyl, cycloalkyl, cycloheteroalkyl, heteroaryl, and aryl in R^{d} are optionally substituted with one to two substituents independently selected from a R^{e};
each R^{e} is selected from halogen, methyl, methoxy, trifluoromethyl, trifluoromethoxy, and hydroxy;
m is selected from 1 and 2;
n is selected from: 0, 1, 2, 3, and 4;
p is selected from 0, 1, and 2;
and pharmaceutically acceptable salts thereof.

6. A compound according to claim 1, of structural formula: 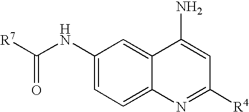
wherein R⁴ and R⁷ are selected according to the table below:

| Ex. # | R⁷ | R⁴ |
|---|---|---|
| 1 | 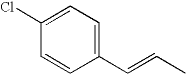 | -n-propyl |
| 2 | 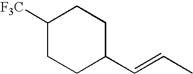 | -n-propyl |
| 3 | 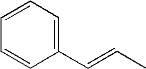 | -n-propyl |
| 4 | 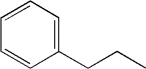 | -n-propyl |
| 5 | 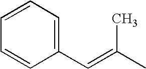 | -n-propyl |
| 6 | 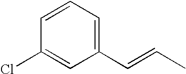 | -n-propyl |
| 7 | 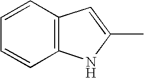 | -n-propyl |
| 8 | 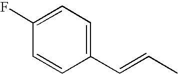 | -n-propyl |
| 9 | 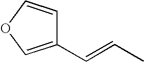 | -n-propyl |
| 10 | 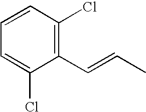 | -n-propyl |
| 11 | 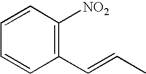 | -n-propyl |
| 12 | 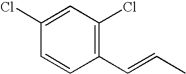 | -n-propyl |
| 13 | 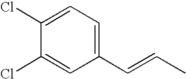 | -n-propyl |
| 14 | 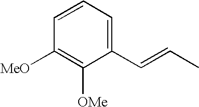 | -n-propyl |
| 15 | 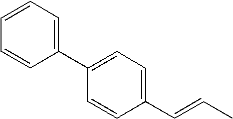 | -n-propyl |
| 16 | 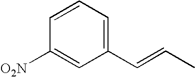 | -n-propyl |
| 17 | 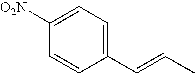 | -n-propyl |
| 18 | 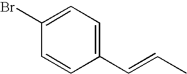 | -n-propyl |
| 19 | 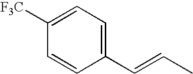 | -n-propyl |
| 20 | 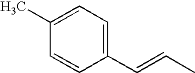 | -n-propyl |
| 21 | 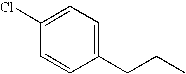 | -n-propyl |
| 22 | 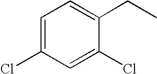 | -n-propyl |
| 23 | 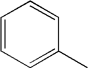 | -n-propyl |
| 24 | 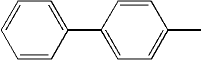 | -n-propyl |
| 25 | 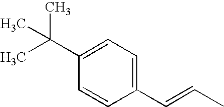 | -n-propyl |
| 26 | 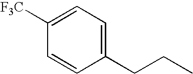 | -n-propyl |
| 27 | 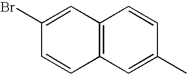 | -n-propyl |
| 28 | 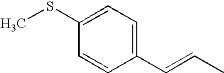 | -n-propyl |
| 29 | 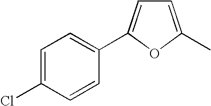 | -n-propyl |
| 30 | 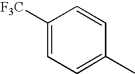 | -n-propyl |
| 31 | 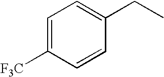 | -n-propyl |
| 32 | 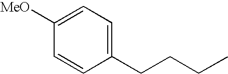 | -n-propyl |
| 33 | 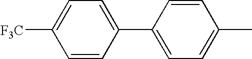 | -n-propyl |
| 34 | 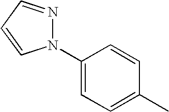 | -n-propyl |
| 35 | 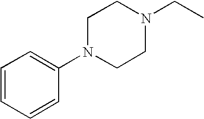 | -n-propyl |
| 36 | 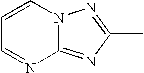 | -n-propyl |
| 37 | 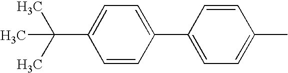 | -n-propyl |
| 38 | 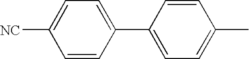 | -n-propyl |
| 39 | 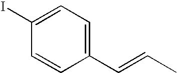 | -n-propyl |
| 40 | 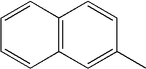 | -n-propyl |
| 41 | 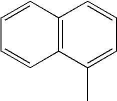 | -n-propyl |
| 42 | 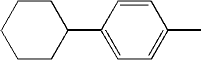 | -n-propyl |
| 43 | 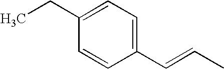 | -n-propyl |
| 44 | 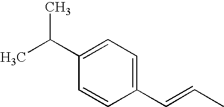 | -n-propyl |
| 45 | 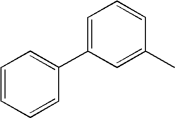 | -n-propyl |
| 46 | 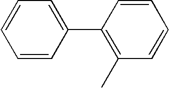 | -n-propyl |
| 47 | 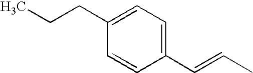 | -n-propyl |
| 48 | 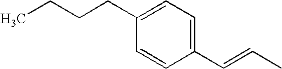 | -n-propyl |
| 49 | 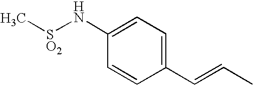 | -n-propyl |
| 50 | 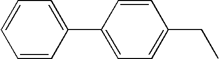 | -n-propyl |
| 51 | 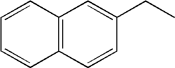 | -n-propyl |
| 52 | 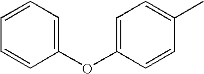 | -n-propyl |
| 53 | 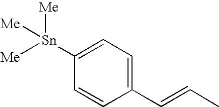 | -n-propyl |
| 54 | 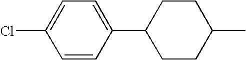 | -n-propyl |
| 55 | 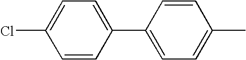 | -n-propyl |
| 56 | 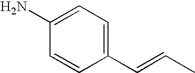 | -n-propyl |
| 57 | 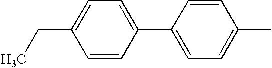 | -n-propyl |
| 58 | 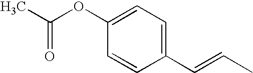 | -n-propyl |
| 59 | 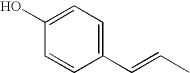 | -n-propyl |
| 60 | 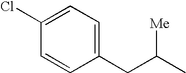 | -n-propyl |
| 61 | 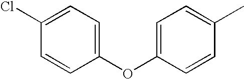 | -n-propyl |
| 62 | 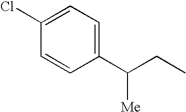 | -n-propyl |
| 63 | 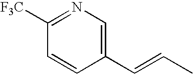 | -n-propyl |
| 64 | 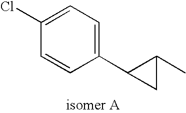 | -n-propyl |
| 65 | 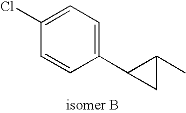 | -n-propyl |
| 66 | 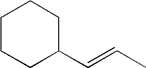 | -n-propyl |
| 67 | 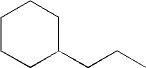 | -n-propyl |
| 68 | 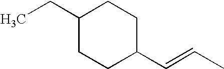 | -n-propyl |
| 69 | 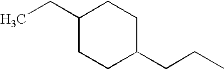 | -n-propyl |
| 70 | 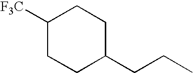 | -n-propyl |
| 71 | 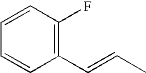 | -n-propyl |
| 72 | 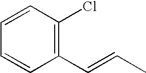 | -n-propyl |
| 73 | 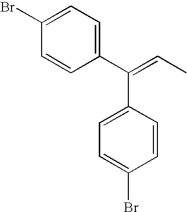 | -n-propyl |
| 74 | 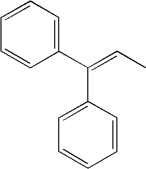 | -n-propyl |
| 75 | 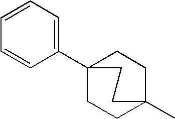 | -n-propyl |
| 76 | 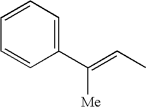 | -n-propyl |
| 77 | 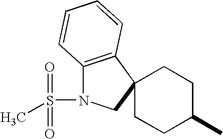 | -n-propyl |
| 78 | 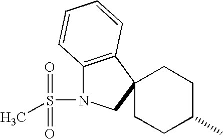 | 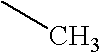 |
| 79 | 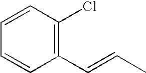 | 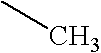 |
| 80 | 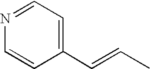 | 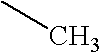 |
| 81 | 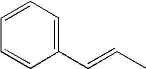 | 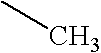 |
| 82 | 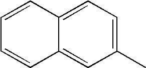 | 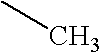 |
| 83 | 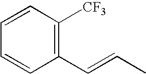 | 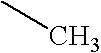 |
| 84 | 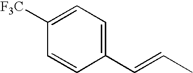 | 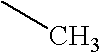 |
| 85 | 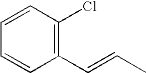 | 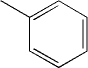 |
| 86 | 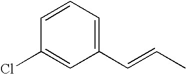 | 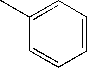 |
| 87 | 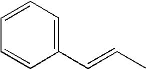 | 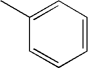 |
| 88 | 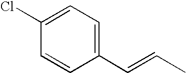 | 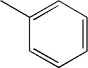 |
| 89 | 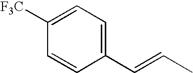 | 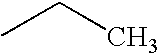 |
| 90 | 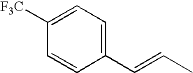 | 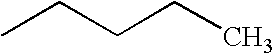 |
| 91 | 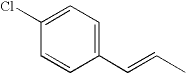 | 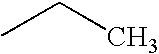 |
| 92 | 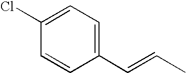 | 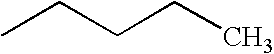 |
| 93 | 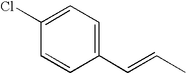 | 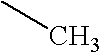 |
| 94 | 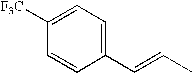 | 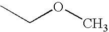 |
| 95 | 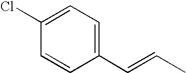 | 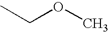 |
| 96 | 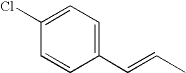 | 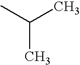 |
| 97 | 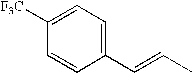 | 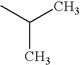 |
| 98 | 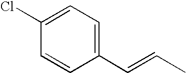 | 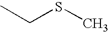 |
| 99 | 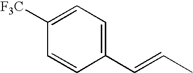 | 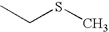 |
| 100 | 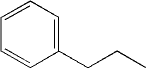 | 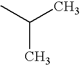 |
| 101 | 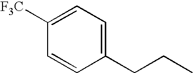 | 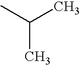 |
| 102 | 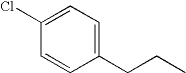 | 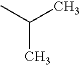 |
| 103 | 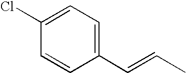 | 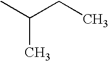 |
| 104 | 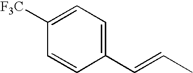 | 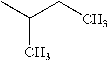 |
| 105 | 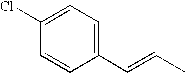 | 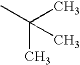 |
| 106 | 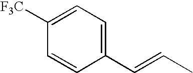 | 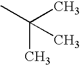 |
| 107 | 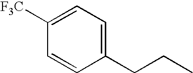 | 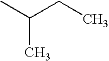 |
| 108 | 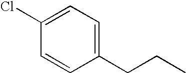 | 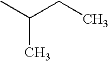 |
| 109 | 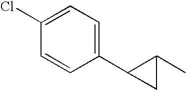 | 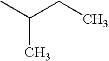 |
| 110 | 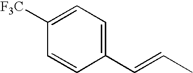 | 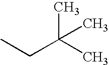 |
| 111 | 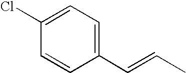 | 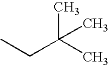 |
| 112 | 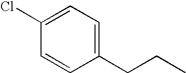 | 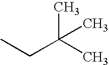 |
| 113 | 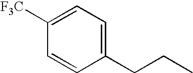 | 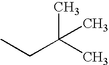 |
| 114 | 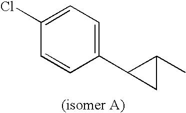 | 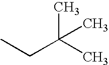 |
| 115 | 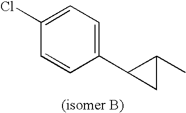 | 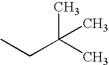 |
| 116 | 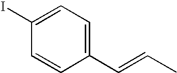 | 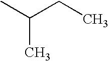 |
| 117 | 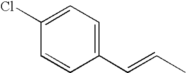 | 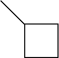 |
| 118 | 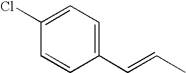 | 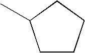 |
| 119 | 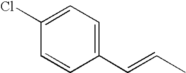 | 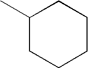 |
| 120 | 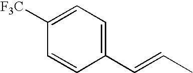 | 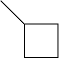 |
| 121 | 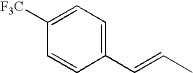 | 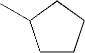 |
| 122 | 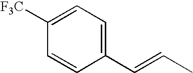 | 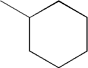 |
| 123 | 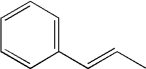 | 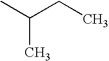 |
| 124 | 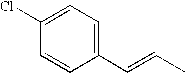 | 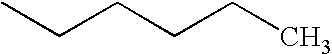 |
| 125 | 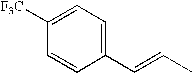 | 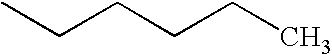 |
| 126 | 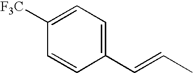 | 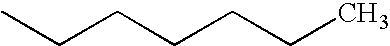 |
| 127 | 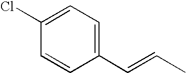 | 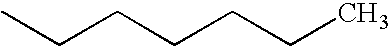 |

and pharmaceutically acceptable salts thereof

7. A compound according to claim 1, of structural formula: 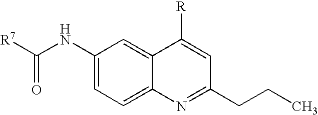
wherein —R⁷ and —R are selected according to the table below:

| Ex. # | R⁷ | R = -NR¹R² |
|---|---|---|
| 128 | 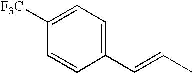 | 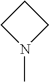 |
| 129 | 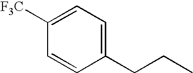 | 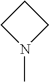 |
| 130 | 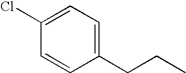 | 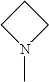 |
| 131 | 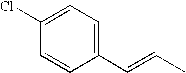 | 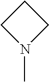 |
| 132 | 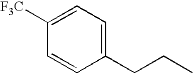 | 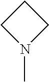 |
| 133 | 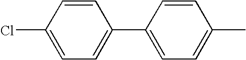 | 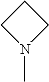 |
| 134 | 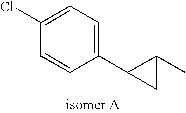 | 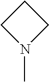 |
| 135 | 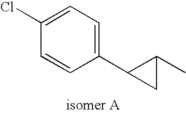 | 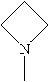 |
| 136 | 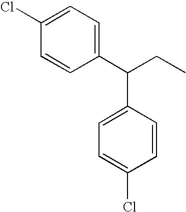 | 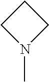 |
| 137 | 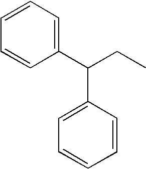 | 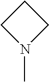 |
| 138 | 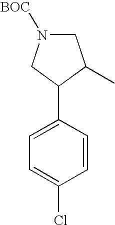 | 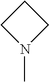 |
| 139 | 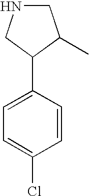 | 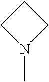 |
| 140 | 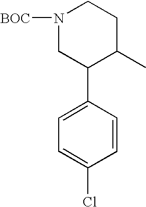 | 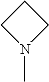 |
| 141 | 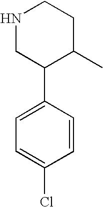 | 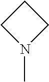 |
| 142 | 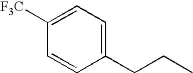 | 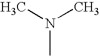 |
| 143 | 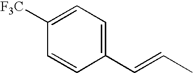 | 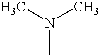 |
| 144 | 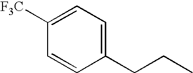 | 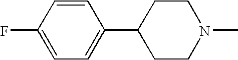 |
| 145 | 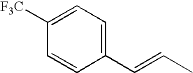 | 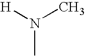 |
| 146 | 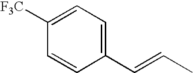 | 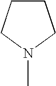 |
| 147 | 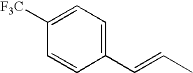 | 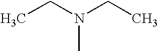 |
| 148 | 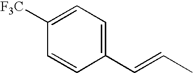 | 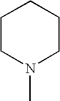 |
| 149 | 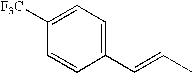 | 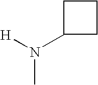 |
| 150 | 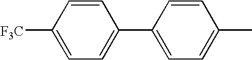 | 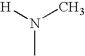 |
| 151 | 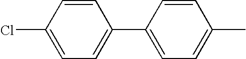 | 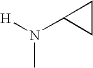 |
| 152 | 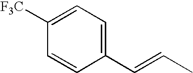 | 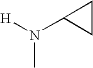 |
| 153 | 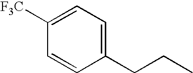 | 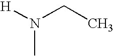 |
| 154 | 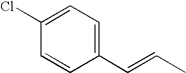 | 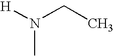 |
| 155 | 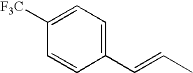 | 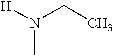 |

and pharmaceutically acceptable salts thereof.

8. The compound according to claim 1 which is selected from the following:

| Ex. # | Structure |
|---|---|
| 156 | 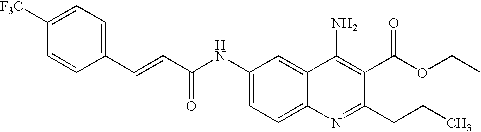 |
| 157 | 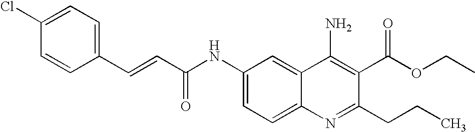 |
| 158 | 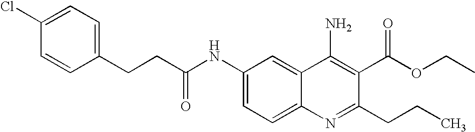 |
| 159 | 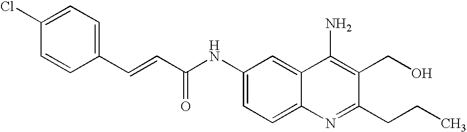 |
| 160 | 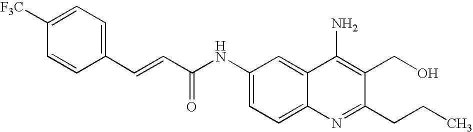 |
| 161 | 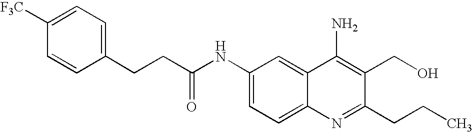 |
| 162 | 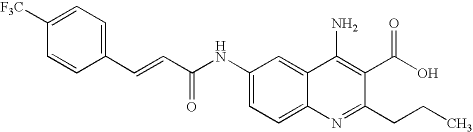 |
| 163 | 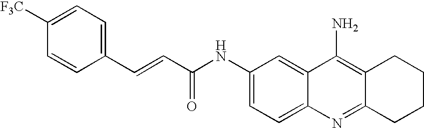 |
| 164 | 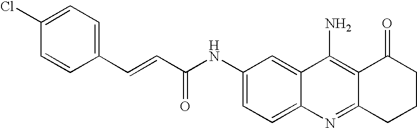 |
| 165 | 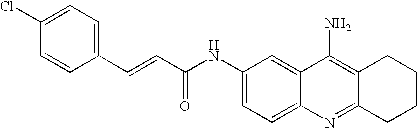 |
| 166 | 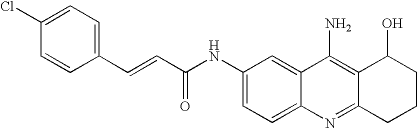 |

and pharmaceutically acceptable salts thereof.

9. The compound according to claim 1, of structural formula: 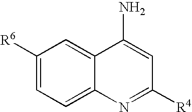
wherein R⁶ and R⁴ are selected according to the table below:

| Ex. # | R⁶ | R⁴ |
|---|---|---|
| 167 | 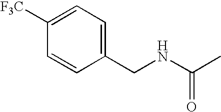 | 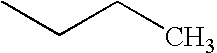 |
| 168 | 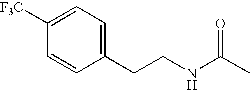 | 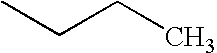 |
| 169 | 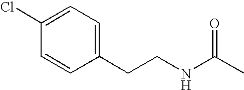 | 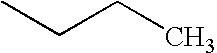 |
| 170 | 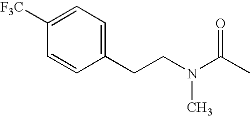 | 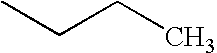 |
| 171 | 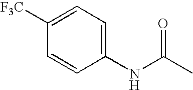 | 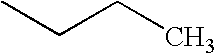 |
| 172 | 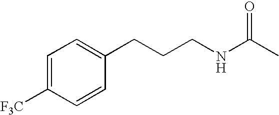 | 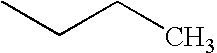 |
| 173 | 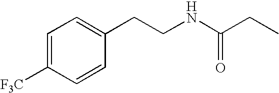 | 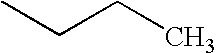 |
| 174 | 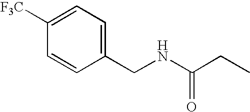 | 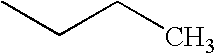 |
| 175 | 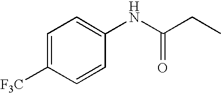 | 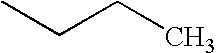 |
| 176 | 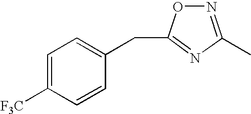 | 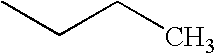 |
| 177 | 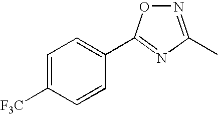 | 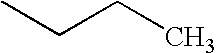 |
| 178 | 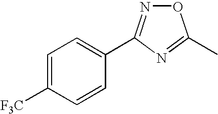 | 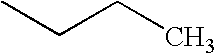 |
| 179 | 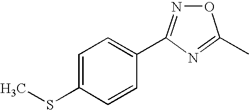 | 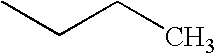 |
| 180 | 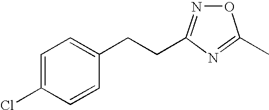 | 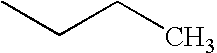 |
| 181 | 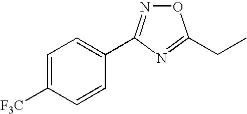 | 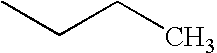 |
| 182 | 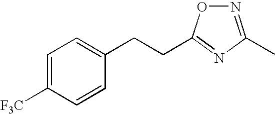 | 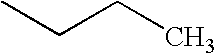 |
| 183 | 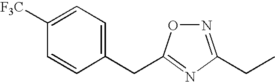 | 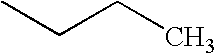 |
| 184 | 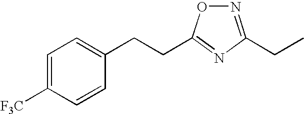 | 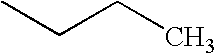 |
| 185 | 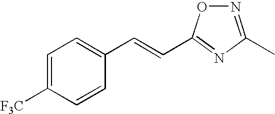 | 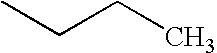 |
| 186 | 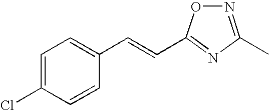 | 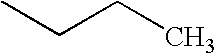 |
| 187 | 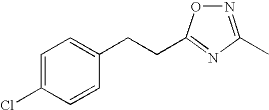 | 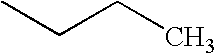 |
| 188 | 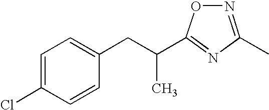 | 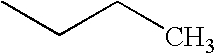 |
| 189 | 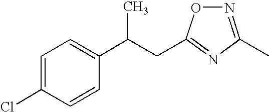 | 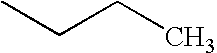 |
| 190 | 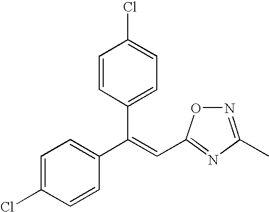 | 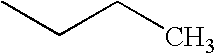 |
| 191 | 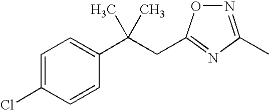 | 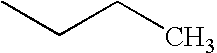 |
| 192 | 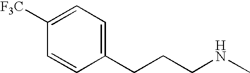 | 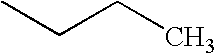 |
| 193 | 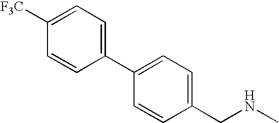 | 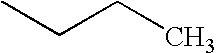 |
| 194A | 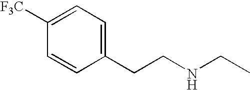 | 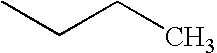 |
| 194B | 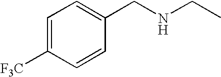 | 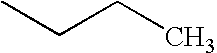 |
| 195 | 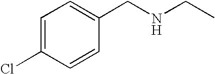 | 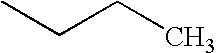 |
| 196 | 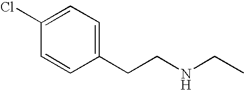 | 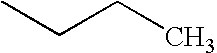 |
| 197 | 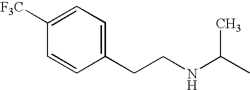 | 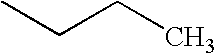 |
| 198 | 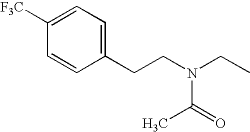 | 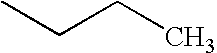 |
| 199 | 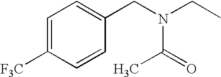 | 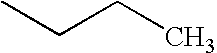 |
| 200 | 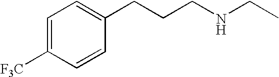 | 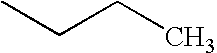 |
| 201 | 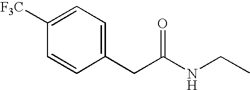 | 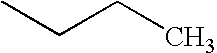 |
| 202 | 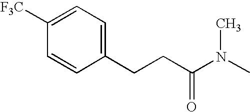 | 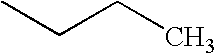 |
| 203 | 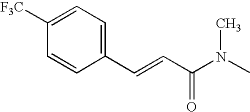 | 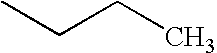 |
| 204 | 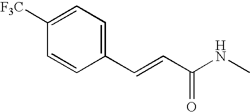 | 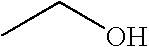 |
| 205 | 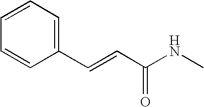 | 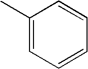 |
| 206 | 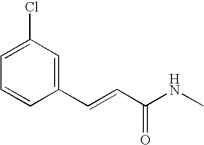 | 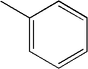 |
| 207 | 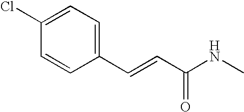 | 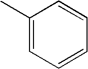 |
| 208 | 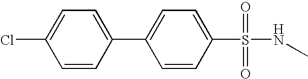 | 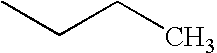 |
| 209 | 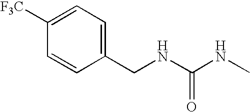 | 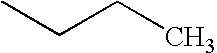 |
| 210 | 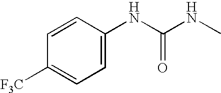 | 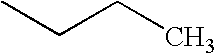 |
| 211 | 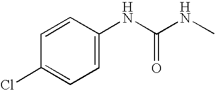 | 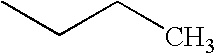 |
| 212 | 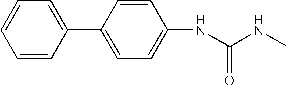 | 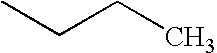 |
| 213 | 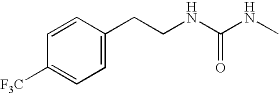 | 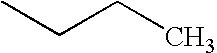 |
| 214 | 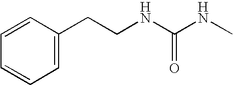 | 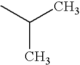 |
| 215 | 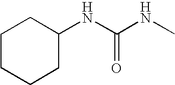 | 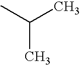 |
| 216 | 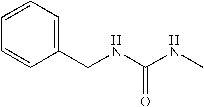 | 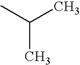 |
| 217 | 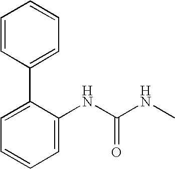 | 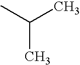 |
| 218 | 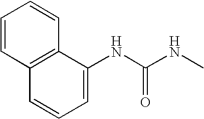 | 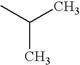 |
| 219 | 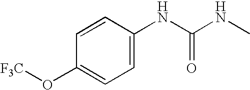 | 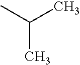 |
| 220 | 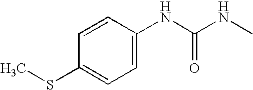 | 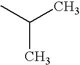 |
| 221 | 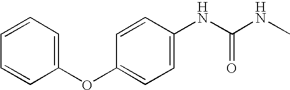 | 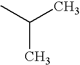 |
| 222 | 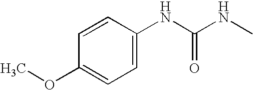 | 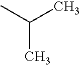 |
| 223 | 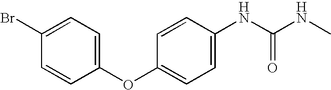 | 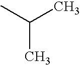 |
| 224 | 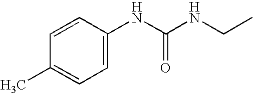 | 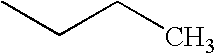 |
| 225 | 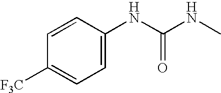 | 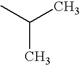 |
| 226 | 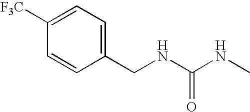 | 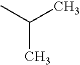 |
| 227 | 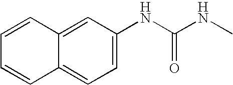 | 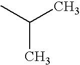 |
| 228 | 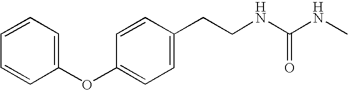 | 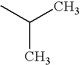 |
| 229 | 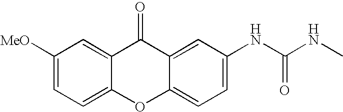 | 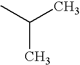 |
| 230 | 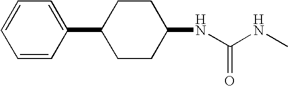 | 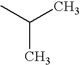 |
| 231 | 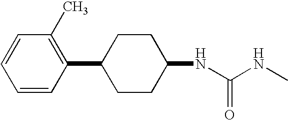 | 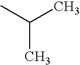 |
| 232 | 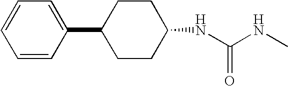 | 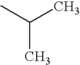 |
| 233 | 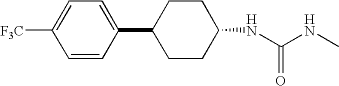 | 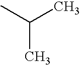 |
| 234 | 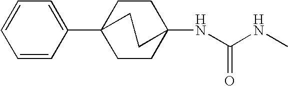 | 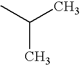 |
| 235 | 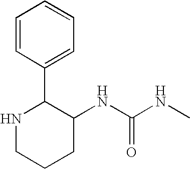 | 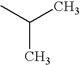 |
| 236 | 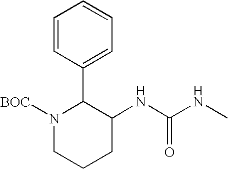 | 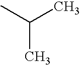 |
| 237 | 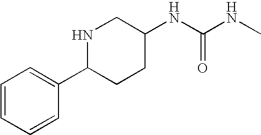 | 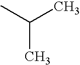 |
| 238 | 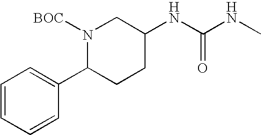 | 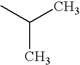 |
| 239 | 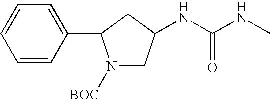 | 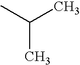 |
| 240 | 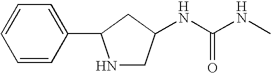 | 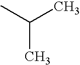 |
| 241 | 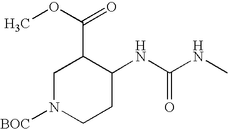 | 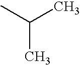 |
| 242 | 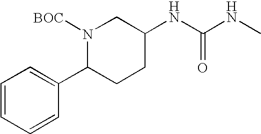 | 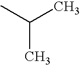 |
| 243 | 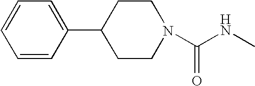 | 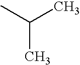 |
| 244 | 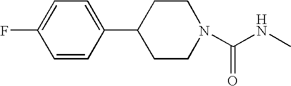 | 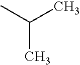 |

10. The compound according to claim 1, selected from the group consisting of:
(1) (2E)-N-(4-amino-2-propylquinolin-6-yl)-3-(4-chlorophenyl)prop-2-enamide,
(2) (2E)-N-(4-amino-2-propylquinolin-6-yl)-3-(2,4-dichlorophenyl)prop-2-enamide,
(3) (2E)-N-(4-amino-2-propylquinolin-6-yl)-3-(1,1′-biphenyl-4-yl)prop-2-enamide,
(4) (2E)-N-(4-amino-2-propylquinolin-6-yl)-3-(4-bromophenyl)prop-2-enamide,
(5) (2E)-N-(4-amino-2-propylquinolin-6-yl)-3-[4-(trifluoromethyl)phenyl]prop-2-enamide,
(6) (2E)-N-(4-amino-2-propylquinolin-6-yl)-3-(4-methylphenyl)prop-2-enamide,
(7) N-(4-amino-2-propylquinolin-6-yl)-1,1′-biphenyl-4-carboxamide,
(8) (2E)-N-(4-amino-2-propylquinolin-6-yl)-3-[4-(methylthio)phenyl]prop-2-enamide,
(9) (2E)-N-[4-(dimethylamino)-2-propylquinolin-6-yl]-3-[4-(trifluoromethyl)phenyl]prop-2-enamide,
(10) N-(4-amino-2-propylquinolin-6-yl)-4′-(trifluoromethyl)-1,1′-biphenyl-4-carboxamide,
(11) (2E)-N-(4-amino-2-propylquinolin-6-yl)-3-(4-iodophenyl)prop-2-enamide,
(12) (2E)-N-(4-azetidin-1-yl-2-propylquinolin-6-yl)-3-[4-(trifluoromethyl)phenyl]prop-2-enamide,
(13) (2E)-N-[4-(methylamino)-2-propylquinolin-6-yl]-3-[4-(trifluoromethyl)phenyl]prop-2-enamide,
(14) (2E)-N-(4-amino-2-ethylquinolin-6-yl)-3-[4-(trifluoromethyl)phenyl]prop-2-enamide,
(15) (2E)-N-(4-amino-2-butylquinolin-6-yl)-3-[4-(trifluoromethyl)phenyl]prop-2-enamide,
(16) (2E)-N-(4-amino-2-ethylquinolin-6-yl)-3-(4-chlorophenyl)prop-2-enamide,
(17) (2E)-N-(4-amino-2-butylquinolin-6-yl)-3-(4-chlorophenyl)prop-2-enamide,
(18) N-(4-azetidin-1-yl-2-propylquinolin-6-yl)-3-[4-(trifluoromethyl)phenyl]propanamide,
(19) (2E)-N-(4-amino-2-propylquinolin-6-yl)-3-(4-ethylphenyl)prop-2-enamide,
(20) (2E)-N-(4-amino-2-propylquinolin-6-yl)-3-(4-isopropylphenyl)prop-2-enamide,
(21) (2E)-N-(4-amino-2-propylquinolin-6-yl)-3-(4-propylphenyl)prop-2-enamide,
(22) N-[4-amino-3-(hydroxymethyl)-2-propylquinolin-6-yl]-3-[4-(trifluoromethyl)phenyl]propanamide,
(23) (2E)-N-[4-amino-2-(methoxymethyl)quinolin-6-yl]-3-[4-(trifluoromethyl)phenyl]prop-2-enamide,
(24) (2E)-N-(4-amino-2-hexylquinolin-6-yl)-3-[4-(trifluoromethyl)phenyl]prop-2-enamide,
(25) (2E)-N-[4-amino-2-(methoxymethyl)quinolin-6-yl]-3-(4-chlorophenyl)prop-2-enamide,
(26) (2E)-N-(4-amino-2-pentylquinolin-6-yl)-3-[4-(trifluoromethyl)phenyl]prop-2-enamide,
(27) (2E)-N-(4-amino-2-pentylquinolin-6-yl)-3-(4-chlorophenyl)prop-2-enamide,
(28) (2E)-N-(4-amino-2-hexylquinolin-6-yl)-3-(4-chlorophenyl)prop-2-enamide,
(29) N-(4-amino-2-propylquinolin-6-yl)-4-(4-chlorophenyl)cyclohexanecarboxamide,
(30) N-(4-amino-2-propylquinolin-6-yl)-4′-chloro-1,1′-biphenyl-4-carboxamide,
(31) N-[4-(methylamino)-2-propylquinolin-6-yl]-4′-(trifluoromethyl)-1,1′-biphenyl4-carboxamide,
(32) N-(4-amino-2-propylquinolin-6-yl)-4′-ethyl-1,1′-biphenyl-4-carboxamide,
(33) (2E)-N-(4-amino-2-isopropylquinolin-6-yl)-3-(4-chlorophenyl)prop-2-enamide,
(34) (2E)-N-(4-amino-2-isopropylquinolin-6-yl)-3-[4-(trifluoromethyl)phenyl]prop-2-enamide,
(35) N-(4-amino-2-isopropylquinolin-6-yl)-3-[4-(trifluoromethyl)phenyl]propanamide,
(36) (2E)-N-(4-amino-2-propylquinolin-6-yl)-3-[6-(trifluoromethyl)pyridin-3-yl]prop-2-enamide,
(37) (2E)-N-(4-azetidin-1-yl-2-propylquinolin-6-yl)-3-(4-chlorophenyl)prop-2-enamide,
(38) N-(4-azetidin-1-yl-2-propylquinolin-6-yl)-4′-chloro-1,1′-biphenyl-4-carboxamide,
(39) (2E)-N-(9-amino-8-oxo-5,6,7,8-tetrahydroacridin-2-yl)-3-(4-chlorophenyl)prop-2-enamide,
(40) (2E)-N-[4-amino-2-(hydroxymethyl)quinolin-6-yl]-3-[4-(trifluoromethyl)phenyl]prop-2-enamide,
(41) (2E)-N-(9-amino-5,6,7,8-tetrahydroacridin-2-yl)-3-(4-chlorophenyl)prop-2-enamide,
(42) (2E)-N-(9-amino-8-hydroxy-5,6,7,8-tetrahydroacridin-2-yl)-3-(4-chlorophenyl)prop-2-enamide,
(43) (2E)-N-(9-amino-5,6,7,8-tetrahydroacridin-2-yl)-3-[4-(trifluoromethyl)phenyl]prop-2-enamide,
(44) (2E)-N-(4-amino-2-sec-butylquinolin-6-yl)-3-(4-chlorophenyl)prop-2-enamide,
(45) (2E)-N-(4-amino-2-sec-butylquinolin-6-yl)-3-[4-(trifluoromethyl)phenyl]prop-2-enamide,
(46) (2E)-3-(4-chlorophenyl)-N-[4-(ethylamino)-2-propylquinolin-6-yl]prop-2-enamide,
(47) (2E)-N-[4-(ethylamino)-2-propylquinolin-6-yl]-3-[4-(trifluoromethyl)phenyl]prop-2-enamide,
(48) (2E)-N-(4-amino-2-tert-butylquinolin-6-yl)-3-(4-chlorophenyl)prop-2-enamide,
(49) (2E)-N-(4-amino-2-tert-butylquinolin-6-yl)-3-[4-(trifluoromethyl)phenyl]prop-2-enamide,
(50) N-(4-amino-2-sec-butylquinolin-6-yl)-3-[4-(trifluoromethyl)phenyl]propanamide,
(51) (2E)-N-(4-amino-2-neopentylquinolin-6-yl)-3-[4-(trifluoromethyl)phenyl]prop-2-enamide,
(52) N-(4-amino-2-isopropylquinolin-6-yl)-N′-(4-phenoxyphenyl)urea
(53) (2E)-N-(4-amino-2-propylquinolin-6-yl)-3-(4-ethylcyclohexyl)prop-2-enamide,
(54) (2E)-N-(4-amino-2-sec-butylquinolin-6-yl)-3-(4-iodophenyl)prop-2-enamide,
(55) N-(4-amino-2-isopropylquinolin-6-yl)-N′-(4-phenylcyclohexyl)urea,
(56) N-(4-amino-2-isopropylquinolin-6-yl)-N′-(2-naphthyl)urea,
(57) (2E)-N-(4-amino-2-cyclobutylquinolin-6-yl)-3-(4-chlorophenyl)prop-2-enamide,
(58) (2E)-N-(4-amino-2-cyclopentylquinolin-6-yl)-3-(4-chlorophenyl)prop-2-enamide,
(59) (2E)-N-(4-amino-2-cyclohexylquinolin-6-yl)-3-(4-chlorophenyl)prop-2-enamide,
(60) (2E)-N-(4-amino-2-cyclobutylquinolin-6-yl)-3-[4-(trifluoromethyl)phenyl]prop-2-enamide,
(61) (2E)-N-(4-amino-2-cyclopentylquinolin-6-yl)-3-[4-(trifluoromethyl)phenyl]prop-2-enamide,
(62) (2E)-N-(4-amino-2-cyclohexylquinolin-6-yl)-3-[4-(trifluoromethyl)phenyl]prop-2-enamide,
(63) (2E)-N-(4-amino-2-methylquinolin-6-yl)-3-[4-(trifluoromethyl)phenyl]prop2-enamide,
(64) 2-propyl-6-(5-{2-[4-(trifluoromethyl)phenyl]ethyl }-1,2,4-oxadiazol-3-yl)quinolin-4-amine,
and pharmaceutically acceptable salts thereof.

11. The compound according to claim 10 selected from:
(1) (2E)-N-(4-amino-2-propylquinolin-6-yl)-3-(4-chlorophenyl)prop-2-enamide,
(2) (2E)-N-(4-amino-2-propylquinolin-6-yl)-3-[4-(trifluoromethyl)phenyl]prop-2-enamide,
(3) (2E)-N-[4-(dimethylamino)-2-propylquinolin-6-yl]-3-[4-(trifluoromethyl)phenyl]prop-2-enamide,
(4) (2E)-N-(4-amino-2-propylquinolin-6-yl)-3-(4-iodophenyl)prop-2-enamide,
(5) (2E)-N-(4-azetidin-1-yl-2-propylquinolin-6-yl)-3-[4-(trifluoromethyl)phenyl]prop-2-enamide,
(6) (2E)-N-[4-(methylamino)-2-propylquinolin-6-yl]-3-[4-(trifluoromethyl)phenyl]prop-2-enamide,
(7) N-(4-azetidin-1-yl-2-propylquinolin-6-yl)-3-[4-(trifluoromethyl)phenyl]propanamide,
(8) (2E)-N-(4-amino-2-propylquinolin-6-yl)-3-(4-ethylphenyl)prop-2-enamide,
(9) (2E)-N-(4-amino-2-propylquinolin-6-yl)-3-(4-isopropylphenyl)prop-2-enamide,
(10) N-(4-amino-2-propylquinolin-6-yl)-4′-chloro-1,1′-biphenyl-4-carboxamide,
(11) N-[4-(methylamino)-2-propylquinolin-6-yl]-4′-(trifluoromethyl)-1,1′-biphenyl-4-carboxamide,
(12) (2E)-N-(4-amino-2-isopropylquinolin-6-yl)-3-(4-chlorophenyl)prop-2-enamide,
(13) (2E)-N-(4-amino-2-isopropylquinolin-6-yl)-3-[4-(trifluoromethyl)phenyl]prop-2-enamide,
(14) N-(4-amino-2-isopropylquinolin-6-yl)-3-[4-(trifluoromethyl)phenyl]propanamide,
(15) (2E)-N-(4-amino-2-propylquinolin-6-yl)-3-[6-(trifluoromethyl)pyridin-3-yl]prop-2-enamide,
(16) (2E)-N-(4-azetidin-1-yl-2-propylquinolin-6-yl)-3-(4-chlorophenyl)prop-2-enamide,
(17) N-(4-azetidin-1-yl-2-propylquinolin-6-yl)4′-chloro-1,1′-biphenyl-4-carboxamide,
(18) (2E)-N-(9-amino-8-oxo-5,6,7,8-tetrahydroacridin-2-yl)-3-(4-chlorophenyl)prop-2-enamide,
(19) (2E)-N-(9-amino-5,6,7,8-tetrahydroacridin-2-yl)-3-[4-(trifluoromethyl)phenyl]prop-2-enamide,
(20) (2E)-N-(4-amino-2-sec-butylquinolin-6-yl)-3-(4-chlorophenyl)prop-2-enamide,
(21) (2E)-N-(4-amino-2-sec-butylquinolin-6-yl)-3-[4-(trifluoromethyl)phenyl]prop-2-enamide,
(22) (2E)-3-(4-chlorophenyl)-N-[4-(ethylamino)-2-propylquinolin-6-yl]prop-2-enamide,
(23) (2E)-N-[4-(ethylarino)-2-propylquinolin-6-yl]-3-[4-(trifluoromethyl)phenyl]prop-2-enamide,
(24) (2E)-N-(4-amino-2-tert-butylquinolin-6-yl)-3-(4-chlorophenyl)prop-2-enamide,
(25) (2E)-N-(4-amino-2-tert-butylquinolin-6-yl)-3-[4-(trifluoromethyl)phenyl]prop-2-enamide,
(26) N-(4-amino-2-sec-butylquinolin-6-yl)-3-[4-(trifluoromethyl)phenyl]propanamide,
(27) N-(4-amino-2-isopropylquinolin-6-yl)-N′-(4-phenoxyphenyl)urea
(28) (2E)-N-(4-amino-2-sec-butylquinolin-6-yl)-3-(4-iodophenyl)prop-2-enamide,
(29) (2E)-N-(4-amino-2-cyclobutylquinolin-6-yl)-3-(4-chlorophenyl)prop-2-enamide,
(30) (2E)-N-(4-amino-2-cyclopentylquinolin-6-yl)-3-(4-chlorophenyl)prop-2-enamide,
(31) (2E)-N-(4-amino-2-cyclohexylquinolin-6-yl)-3-(4-chlorophenyl)prop-2-enamide,
(32) (2E)-N-(4-amino-2-cyclobutylquinolin-6-yl)-3-[4-(trifluoromethyl)phenyl]prop-2-enamide,
(33) (2E)-N-(4-amino-2-cyclopentylquinolin-6-yl)-3-[4-(trifluoromethyl)phenyl]prop-2-enamide,
(34) (2E)-N-(4-amino-2-cyclohexylquinolin-6-yl)-3-[4-(trifluoromethyl)phenyl]prop-2-enamide,
(35) (2E)-N-(4-amino-2-methylquinolin-6-yl)-3-[4-(trifluoromethyl)phenyl]prop-2-enamide,
(36) 2-propyl-6-(5-{2-[4-(trifluoromethyl)phenyl]ethyl}-1,2,4-oxadiazol-3-yl)quinolin-4-amine,
and pharmaceutically acceptable salts thereof.

12. A method of treating or suppressing a disease mediated by the MCH receptor in a subject in need thereof comprising administeration of a therapeutically effective amount of a compound according to claim 1.

13. The method according to claim 12 wherein the disease is mediated by the MCHLR receptor.

14. The method according to claim 12 wherein the disease mediated by the MCH receptor is selected from: obesity, diabetes, appetite and eating disorders, cardiovascular disease, hypertension, dyslipidemia, myocardial infarction, gall stones, osteoarthritis, certain cancers, AIDS wasting, cachexia, frailty (particularly in elderly), binge eating disorders including bulimina, anorexia, mental disorders including manic depression, depression, schizophrenia, mood disorders, delirium, dementia, severe mental retardation, anxiety, stress, cognitive disorders, sexual function, reproductive function, kidney function, diuresis, locomotor disorders, attention deficit disorder (ADD), substance abuse disorders and dyskinesias including Parkinson's disease, Parkinson-like syndromes, Tourette's syndrome, Huntington's disease, epilepsy, improving memory function, and spinal muscular atrophy.

15. A method of treating obesity in a subject in need thereof comprising administration of a therapeutically effective amount of a compound according to claim 1.

16. The method according to claim 15, additionally comprising administration of a therapeutically effective amount of an anorectic agent or a selective serotonin reuptake inhibitor.

17. The method according to claim 16 wherein: the anorectic agent is selected from: aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; and the selective serotonin reuptake inhibitor is selected from: fluoxetine, fluvoxamine, paroxetine and sertraline.

18. A method of preventing obesity in a person at risk for obesity comprising administration to said person of about 0.01 mg to about 100 mg per kg of a compound according to claim 1.

19. A composition comprising a compound according to claim 1 and a pharmaceutically acceptable carrier.

20-21. (cancelled).

22. A method of treating a condition selected from schizophrenia, bipolar disorder and depression in a subject in need thereof comprising administering an effective amount of an MCH-1R receptor antagonist compound to the subject.

23. A method of treating depression in a subject in need thereof comprising administering an effective amount of an MCH-1R receptor antagonist compound according to claim 1 to the subject.
